(19) United States
(12) Patent Application Publication
(10) Pub. No.: US 2010/0074920 A1
(43) Pub. Date: Mar. 25, 2010

(54) PEPTIDE VACCINE FOR INFLUENZA VIRUS

(75) Inventors: Jari Natunen, Vantaa (FI); Jukka Hiltunen, Helsinki (FI); Ritva Niemela, Helsinki (FI); Jari Helin, Vantaa (FI); Olli Aitio, Helsinki (FI)

Correspondence Address: BROOKS KUSHMAN P.C., 1000 TOWN CENTER, TWENTY-SECOND FLOOR, SOUTHFIELD, MI, 48075, US

(73) Assignee: GLYKOS FINLAND OY, Helsinki (FI)

(21) Appl. No.: 12/447,018

(22) PCT Filed: Oct. 26, 2007
(86) PCT No.: PCT/FI2007/050577

§ 371 Date: Nov. 6, 2009

(30) Foreign Application Priority Data
Oct. 26, 2006 (FI) 20060946

(51) Int. Cl. A61K 39/145; C07K 14/11; C07H 21/04; G01N 33/53; C12Q 1/70; A61P 37/04; A61P 31/12

(52) U.S. Cl. 424/209.1; 436/86; 530/329; 530/328; 536/23.72; 436/94; 435/5

(57) ABSTRACT

The invention relates to the method for evaluating the potential of a chemical entity, such as an antibody, to bind to a peptide epitope derived from the divalent sialoside binding site of hemagglutinin protein of influenza virus. The invention also provides peptide epitopes 5 for use in the prevention and/or treatment of influenza or for the development of such treatment or vaccine against influenza.

The invention relates to the method for evaluating the potential of a chemical entity, such as an antibody, to bind to a peptide epitope derived from the divalent sialoside binding site of hemagglutinin protein of influenza virus. The invention also provides peptide epitopes for use in the prevention and/or treatment of influenza or for the development of such treatment or vaccine against influenza.

### BACKGROUND OF THE INVENTION

Influenza virus infect the airways of a patient and initially cause general respiratory symptoms, which may result in high morbidity and mortality rates, especially in elderly persons. Thus, good targets for attacking the virus are constantly searched for. The significance of hemagglutinin protein of influenza virus in the pathogenesis of the virus has been known for a relatively long time. Consequently, in the field of vaccine and antibody development an aim has been to develop vaccines against conserved regions of influenza virus hemagglutinins. For example, a patent application of Takara Shuzo (EP0675199) describes antibodies which recognizes the stem region of certain influenza virus subtypes. WO0032228 describes vaccines containing hemagglutinin epitope peptides 91-108, 307-319, 306-324 and for non-caucasian populations peptide 458-467. Lu et al. 2002 describe a conserved site 92-105. Lin and Cannon 2002 describes conserved residues Y88, T126, H174, E181, L185 and G219. Hennecke et al. 2000 studied complex of hemagglutinin peptide HA306-318 with T-cell receptor and a HLA-molecule. Some conserved peptide structures have been reported in the primary binding site and a mutation which changes the binding specificity from α6-sialic acids to α3-sialic acids.

There is development of vaccines against different peptides of influenza hemagglutinin on different or partially overlapping sites. An example of different site is the cleavage site of hemagglutinin HA₀ including, e.g., ones developed by Merck and Biondvax. Other development including minor part of somewhat overlapping hemagglutinin peptides including ones developed by Variation biotechnology, e.g., including peptide 1 and peptide 4 described in WO06128294 (Jul. 12, 2006) and Biondvax including peptide HA91 (e.g. WO07066334, 14.6.07) directed to longer peptides epitopes which are not conformational and conjugated as disclosed in the present invention.

Certain MHCII T-cell epitope peptides directed publications disclosed as prior art for our earlier application PCT/FI2006/050157 were: US 2006002947(D1), WO9859244 (D2), Gelder C et al In Immun. (1998) 10, 211-222 (D3), and D4 J. Virol 1991 65 364-372. Based on the length of the peptides from mouse models and multitude of peptides from which there are varying and partially contradicting results endless number of small peptides could be derived, but the effective definitive peptide epitopes cannot be known.

Targeting virus surface and carbohydrate binding site. The above publications D1-D4 are not targeted to epitopes present only on the surface and on the carbohydrate binding site of the influenza virus. The long sequences are randomly derived from influenza virus and are only partially available for recognition on the surface of virus. It is realized that any immune reaction (cell mediated or antibody mediated) against influenza are useless and misdirected, when not targeted against the surface of the virus proteins, and the result cannot be as good as disclosed in the present invention.

Conserved epitopes. The publications D1-D4 are directed to long peptides specific for single type of influenza virus while present invention is directed to conserved peptide epitopes allowing directing immune reaction to multiple virus strains of major human virus such as H1, H3 or H5 and relevant semiconserved variants thereof. It is realized that misdirected effect against long epitope (as described above) against a single strain is not as useful as the multi strain specific effect according to the invention.

Prior art D1-D4 do not include peptides recognized by antibodies but obligatorily larger MHCII-peptides. The publications D1-D4 describe so called MHCII-receptor mediated, T-cell immune reactions, which are different from the antibody mediated reactions according to the invention. It is obvious to anyone skilled in the art peptide epitopes according to the invention, which are immunogenic and cause antibody mediated immune reactions in human, cannot be known from the publications directed to different larger peptides and cell mediated immunity. D1-D4 describing large peptides binding to T-cell receptors. The recognition of peptides by T-cell receptors, as indicated in D-publications, would require large peptides, it is indicated in D2 that MHCII binding requires 13-20 amino acid residues (D2 page 1 lines 33-35). All the peptides of D1-D4 are in this range.

Cost and productability. It is further obvious that it is much cheaper, robust and controllable to produce short than long peptides.

Antibody mediated immune responses. The antigenicity of peptide with regard to antibody mediated immune response depend on recognition of the peptides by variable regions of antibodies coded by specific antibody genes (V-, D-, and J-segments) in B-cells (Roitt, Brostoff and Male Immunology fourth edition 1996, or any equivalent general text book). It is obvious that this cannot be determined from T-cells receptor bindings such as indicated in background The invention revealed that the short peptide epitopes are immunogenic and related to antibody mediated protection against human influenza infection. The present invention indicates antibody mediated immune responses, that are especially useful against influenza.

Analytic use against human natural antibodies. It is further realized that the long peptides suggested in D1-D4 do not reveal usefulness of the present short peptide epitopes in analysis of human antibody mediated immune reactions against the carbohydrate binding site of hemagglutinin. The invention revealed that there are individual specific differences in immune reactions against the peptides and these correlate to the structures of various influenza virus strains to which the test subject would have been exposed to.

There is development of vaccines against other proteins of influenza such as M2 protein or peptide epitopes are developed by the companies including Merck US (peptides), Acambis (with Flanders Univ.), AlphaVax (with NIH, pandemic), VaxInnate (with Yale Univ.), Dynavax (with support from NIH), Cytos Biotech, CH), GenVec (with NIAID), or Molecular Express, Ligocyte or Globe immune or Biondvax (Israel, Ruth Amon and colleagues) and known from the background of their publications. M2 also referred as M2e is common (conserved) antigen and ion channel on influenza, it is not accessible on viral surface but targeted on infected cells (assembly of virus) and it does not cure effectively but relieve disease (Science 2006, Kaiser) and NP protein (nucleoprotein of influenza) or peptide epitope are developed e.g. by the companies Biondvax, AlphaVax, GenVec and known from the background of their publications)

It appears that the high affinity bindings caused by the polylactosamine backbone allow effective evolutionary changes between different types of terminally sialylated structures. Currently the influenza strains binding to human are more α6-sialic acid specific, but change may occur quickly. Therefore effective medicines against more “zoonotic” influenzas spreading to human from chicken or possibly from ducks need to be developed. There are examples of outbreaks of “chicken influenza” like the notorious Hong Kong-97 strain, which was luckily stopped by slaughtering all chickens in Hong Kong and thus resulted in only a few human casualties. The major fear of authorities such as WHO is the spread of such altered strains avoiding resistance in population based on the previous influenza seasons and leading to global infection, pandemic, of lethal viruses with probable α3-sialic acid binding. A major catastrophy of this type was the Spanish flu in 1918. An outbreak of an easily spreading influenza virus is very difficult to stop. There are currently effective medicines though sialidase inhibitors, if effective also against to non-human sialidases, could be of some use and the present vaccines give only temporary protection.

The present invention is directed to use peptide epitopes and corresponding nucleic acids derived from large sialic acid binding site determined in a previous patent application for analysis and typing of influenza and for therapeutics, especially vaccines and immunogenic medication against influenza viruses, especially human influenza viruses and in another embodiment against influenza viruses of cattle (/or wild animals) including especially pigs, horses, chickens(hens) and ducks. The benefit of the short peptide epitopes is that these direct the immune response precisely to the binding site of influenza and block the spreading of the virus.

In silico screening of ligands for a model structure is disclosed for instance in EP1118619 B1 and WO0181627.

The present invention revealed novel antibody target influenza hemagglutinin peptides, including following properties
1) exposed on the surface of the influenza virus
2) more importantly the peptides are part of carbohydrate binding site of hemagglutinin protein of influenza virus
3) partially conserved and thus useful against multiple strains of influenza
4) cheaper and easier to produce and control
5) not obvious from the longer MHCII-binding peptides, because this interaction requires about 20 meric peptides
6) Based on the very large background of long peptides with varying in vitro data from mainly animal models it is not possible derive effective small epitopes according to the invention. Especially it is not possible known effective short sequences from large peptides comprising tens or hundreds of small epitopes or the exact lengths of the short epitopes.
7) human natural antibodies can recognize the epitopes, animal data is not relevant with regard to human immune system, especially antibodies
8) associated with antibody mediated immune reactions and the antibodies can effectively block the virus adhesion and the disease
9) useful in assays of human natural antibodies
10) Highly immunogenic variants of the peptides involving current influenza types, especially variants,
11) Highly immunogenic variants, which are associated with strong immune reaction in context of vaccination and/or severe influenza infection.
12) The present invention provides especially highly effective conformational presentation involving side chain linked or cyclic conformational structures
13) The present invention effective conjugate structures and polyvalent conjugates for the presentation of the peptides. It is notable that the T-cell directed peptides are especially used as monomeric substances targeting MHC-receptors.
14) Relevant and useful variants and preferred structures among the possible peptides.

It is realized that an antibody mediated immune reaction against such peptide epitope is able to block the binding of the virus and thus stop the infection. It is further realized that it is useful to study antibody mediated immune reactions against the peptides to reveal natural resistance to various types of human infecting influenza viruses.

### SUMMARY OF THE INVENTION

Based on sequence comparison of the HA gene from H1,H3 and H5 sequences a series of primers directed to well conserved regions within these genes has been developed. These primers are useful to screen for a wide variety of HA isolates, and allow for screening, treatment, prevention and/or alleviation of influenza caused symptoms by the peptides and peptide antibodies of the present invention.

These primers are useful for detecting the presence of influenza A virus HA in a sample, for example a sample derived from an organism suspected of carrying such a virus, and may be used in a reverse-transcription polymerase chain reaction in order to detect the presence of virus in the sample. The primers also encompassing peptide regions of the invention help to identify what antibodies or oligosaccharides of the invention to use.

Thus, in another aspect the present invention provides a method for detecting influenza A virus subtypes in a sample comprising amplifying DNA reverse transcribed from RNA obtained from the sample using one or more primers each comprising a sequence of any one of primer sequences; and detecting a product of amplification, wherein the presence of the product of amplification indicates the presence of an influenza virus subtype HA in the sample.

The methods described herein can be used to detect a wide variety of influenza A virus isolates. Using a one-step method, in which RNA is reverse-transcribed and product is amplified in a single reaction tube, allows for a reduction in detection time, minimizes sample manipulation and lowers the risk of cross-contamination of samples. Thus, the described methods using the described primers may be useful for early detection and/or diagnosis of influenza A infection. Furthermore, these methods can be used to determine approximate viral load in a sample, which application is useful hi clinical and public health management settings.

The primers of the invention may be useful in other amplification methods, such as nucleic acid based sequence amplification methods to detect the presence of influenza A virus subtypes in a sample. The primers of the invention may also be useful for sequencing DNA corresponding to the HA gene of influenza A virus subtypes.

In another aspect, there is provided a method of detecting influenza A virus subtypes in a sample comprising contacting the sample with a primer immobilized on a support, said primer comprising a primer sequence under conditions suitable for hybridizing the primer and the sample; and detecting hybridization of the immobilized primer and the sample.

In a further aspect, there is provided a method of influenza A virus subtype in a sample comprising contacting the sample with a nucleic acid microarray, the nucleic acid microarray comprising one or more primers, under conditions suitable for hybridizing the one or more primers and the sample; and detecting hybridization of the one or more primers and the sample.

In another aspect, there is provided a nucleic acid microarray comprising a primer, said primer comprising a sequence of any one of primer sequences annealing to the DNA in or vicinity of peptide sequences of the present invention.

In a further aspect, there is provided a kit comprising a primer as defined herein and instructions for detecting influenza A virus subtype in a sample.

In another aspect, there is provided a treatment method comprising a primer or primers as defined herein, the primer(s) detect a nucleotide encoding a peptide of the invention and identification of the HA type helps to treat a patient with a oligosaccharides or antibodies recognizing peptide epitopes of the present invention.

Other aspects and features of the present invention will become apparent to those of ordinary skill in the art upon review of the following description of specific embodiments of the invention in conjunction with the accompanying figures.

### A BRIEF DESCRIPTION OF FIGURES AND SCHEMES

FIG. 1. The complex structure between influenza virus hemagglutinin and the oligosaccharide 7. Yellow structure indicates the oligosaccharide position. Some key aminoacid residues are marked with red.

FIG. 2. “Top view” of the complex between the oligosaccharide 7 (yellow) and the influenza virus hemagglutinin. The red color indicate nonconserved aminoacids, white the N-glycan, and blue the conserved aminoacid in region close to the binding site.

FIG. 3. “Right side” view of the complex between the oligosaccharide 7 (yellow) and the influenza virus hemagglutinin, the upper structure. The red color indicate nonconserved aminoacids, white the N-glycan, and blue the conserved amino acid in region close to the binding site.

FIG. 4. “Front view of the complex between the oligosaccharide 7 (yellow) and the influenza virus hemagglutinin, the upper structure. The red color indicate nonconserved aminoacids, white the N-glycan, and blue the conserved amino acid in region close to the binding site.

FIG. 5. ELISA assay of serum antibodies of test subjects 1-6 (S1-6) on maleimide immobiliased peptides 1 and 2 and peptide HA11, Y-axis indicates the absorbance units.

FIG. 6. ELISA assay of serum antibodies of test subjects 1-6 (S1-6) on streptavidin immobiliased peptides 1-3, Y-axis indicates the absorbance units.

FIG. 7. Exemplary HA subtypes from human, swine, and avian used for the determination of amino acid variation in peptide regions and sequences of the present invention.

FIG. 8. HA H1 amino acid variation within a peptide 1 and prepeptide and postpeptide regions.

FIG. 9. HA H1 amino acid variation within a peptide 2 and prepeptide and postpeptide regions.

FIG. 10. HA H1 amino acid variation within a peptide 4 and prepeptide and postpeptide regions.

FIG. 11. HA H1-H5 amino acid variation within a peptide 1 and prepeptide and postpeptide regions.

FIG. 12. HA H1 amino acid variation within a peptide 3 and prepeptide and postpeptide regions.

FIG. 13. H1 model sequence used for numbering of H1 primer sequences.

FIG. 14. H3 model sequence used for numbering of H3 primer sequences.

FIG. 15. H5 model sequence used for numbering of H3 primer sequences.

FIG. 16. Alignment between H1N1, H3N2 and H5N1 nucleotide sequences (from FIGS. 13-15).

FIG. 17. Degenerate forward and reverse primers for H1.

FIG. 18. Degenerate forward and reverse primers for H3.

FIG. 19. Degenerate forward and reverse primers for H5. Underlined primers are with 0 degeneracy.

FIG. 20. Peptide sequence epitopess derived from human H1 viruses.

FIG. 21. Peptide sequence epitopess derived from human H3 viruses.

FIG. 22. Peptide sequence epitopess derived from human and animal H1, H2, H3, H4 and H5 viruses.

FIG. 23. ELISA binding assay of serum antibodies of test subjects Serum 1B-8B (S1B-S8B) on streptavidin immobilized peptide 1B. Y-axis indicates absorbance units.

FIG. 24. ELISA binding assay of serum antibodies of test subjects Serum 1B-8B (S1B-S8B) on streptavidin immobilized peptide 2B. Y-axis indicates absorbance units.

FIG. 25. ELISA binding assay of serum antibodies of test subjects Serum 1B-8B (S1B-S8B) on streptavidin immobilized peptide 3B. Y-axis indicates absorbance units.

FIG. 26. ELISA binding assay of serum antibodies of test subjects Serum 1B-8B (S1B-S8B) on streptavidin immobilized peptide 4B. Y-axis indicates absorbance units.

FIG. 27. ELISA binding assay of serum antibodies of test subjects Serum 1B-8B (S1B-S8B) on streptavidin immobilized peptide 5B. Y-axis indicates absorbance units.

FIG. 28. Comparison of ELISA binding assays of serum antibodies of test subjects Serum 1B-8B (S1B-S8B) on streptavidin immobilized peptide 1B and peptide 3. Y-axis indicates absorbance units.

### DETAILED DESCRIPTION OF THE INVENTION

The invention reveals novel peptide vaccine compositions, and peptides for analysis and development of antibodies, when the peptides are derived from carbohydrate binding sites of carbohydrate binding proteins (lectins/adhesions) of pathogens, in a preferred embodiment human pathogens such as influenza virus.

The preferred carbohydrate binding sites are carbohydrate binding sites of pathogens comprising large carbohydrate binding sites involving binding to multiple monosaccharide units, more preferably including binding sites for two sialic acid structures. The invention is specifically directed to use of several peptides derived from carbohydrate binding site(s) of a pathogen surface protein, preferably from different parts of the carbohydrate binding site, more preferably from two different sialic acid epitope binding sites or one sialic acid binding site and conserved/semiconserved carbohydrate binding site bridging the sialic acid binding sites.

The invention reveals that conserved or semiconserved amino acid residues form reasonably conserved peptide epitopes at the binding sites of sialylated glycans, preferably binding sites disclosed in the invention. The preferred peptides are derived from the hemagglutinin protein of human influenza protein. It is realized that these epitopes can be used for development of antibodies and vaccines.

The useful antigenic peptides disclosed in the invention are available on the surface of the pathogen, preferably on viral surface.

The peptides which are 1) derived from the carbohydrate binding site (or in a separate embodiment more generally from a conserved binding site of low molecular weight ligand) and which are 2) present on the surface of a pathogen are referred here as “antigen peptides”.

### Peptide 1, Peptide 2 and Peptide 3

The invention revealed specific linear amino acid sequences from the large carbohydrate binding site of influenza A viruses, which are useful for studies of binding of antibodies, selection of antibodies and immunizations. Furthermore it was revealed that the regions can be effectively analysed from nucleic acid of influenza virus by PCR-methods. In a preferred embodiment the analysis of nucleic acids is used as a first test for defining a new peptide. More preferably, the peptide 1 is conjugated from a residue corresponding to cysteine 97 or peptide 2 is conjugated from a residue corresponding to cysteine 139 as defined by the amino acid sequence of X31-hemagglutinin.

Peptide 1 comprises a hepta peptide epitope core starting from amino acid residue position corresponding to the position 91 of influenza H3×31 sequence and ending at cysteine residue 99.

Examples of peptide epitope core from H3 includes, SKAFSNC in X31, and in recent/current viruses especially SKAYSNC and more rare SKADSNC, and STAYSNC, e.g. Table 9, examples of H1 peptide epitope cores includes NSENGTC, NPENGT, and NSENGIC, e.g. Table 8.

It is realized that the Other influenza virus A hemagglutinins can be aligned with X31 sequence as shown in Figures and Tables.

Peptide 2 comprises a hepta peptide epitope core starting from amino acid residue position corresponding to the position 136 of influenza H3×31 sequence and ending residue 141 including at cysteine residue 139. Examples of peptide 2 epitope core from H3 includes, GSNACKR in X31, and in recent/current viruses especially GSYACKR and more rare recent GSSACKR, e.g. Table 9 and even more recent TSSACKR(R) (e.g. (A/Nagasaki/N01/2005) or, TSSACIR(R) (e.g. A/USA/AF1083/2007) or SSSACKR(R) (e.g. A/Wisconsin/67/2005) examples of H1 peptide epitope cores includes (G)VTAACSH, and (G)VTASCSH, e.g. Table 8 (N-terminal G is preferred additional residue) and more recently (G)VSASCSH (A/Thailand/CU75/2006).

Peptide 3 comprises a hepta peptide epitope core starting from amino acid residue position corresponding to the position 220 of influenza H3×31 sequence and ending residue 226. Examples of peptide 3 epitope core from H3 includes, RPWVRGL in X31, and in recent/current viruses especially RPRVRD(V/I/X)(P), according to the Table 10, where in the last residue is V or I or other residue X and a preferred C-terminal additional residue is P, which is preferred because it affect the conformation of the peptide, in a preferred embodiment or RPRVRNI(P), as in new virus (A/Nagasaki/N01/2005) and RPRIRNI(P) (e.g. A/Wisconsin/67/2005).

Examples of H1 peptide 3 epitope cores includes RPKVRDQ common H1, Table 10. The invention revealed by antibody binding studies that cyclic from comprising the core heptapeptide are especially effective. The preferred peptides 3 further includes homologous H5 virus peptides such as RPKVNGQ and similar as defined in Tables.

In the preferred cyclic form both first additional residues from N-terminus and C-terminus are replaced by cysteine or cysteine analogous residue forming disulfide bridge or analogous structure. The sequence may further comprise additional residues X₄X₃X₂ or Y₂Y₃Y₄ or a sequence of up to 100 amino acid residues, preferably up to 30 residues derived from the influenza hemagglutinin.

The invention is further directed to truncated epitopes of the peptides so that one or two N-terminal and/C-terminal residues are omitted, the preferred peptides comprise preferably a short peptide epitopes of three or four amino acid residues in the middle of sequences, consensus of this sequence can be used for recognition of specific peptide type according to the invention. In a preferred embodiment the peptide epitope comprise additional aminoacid residues according to the invention, such 1-4 amino acid, more preferably 1-3 or even more preferably 1-2 aminoacid residues on N-terminal and/or C-terminal side of the peptide epitope core. The additional aminoacid residues are included with provision, that when the peptide is used as linear peptide without conformational presentation and/or conjugation according to the invention the length of the peptide is preferably 1-2 amino a acid residues or less and as described for the preferred short peptides according to the invention

These additional amino acid residue when derived from consecutive aminoacid residues of influenza virus have function in supporting the conformation of the preferred short peptide epitopes. The peptides may further comprise additional amino acid sequence from influenza virus, especially when the peptides are preferred conformational peptides according to the invention.

General Presentation of the Core Peptide with Additional Residues

The general sequence of the short peptide epitopes according to the invention are

X₄X₃X₂X₁C₁C₂C₃C₄C₄C₅C₆C₇Y₁Y₂Y₃Y₄
wherein C₁C₂C₃C₄C₄C₅C₆C₇ are core peptide epitope core aminoacid residues defined as consessus sequence for specific peptide 1-3 type in the invention, so that the characteristic short (or very short) peptide epitope may be truncated peptide may be truncated by removing one or two of C₁C₂ and C₆C₇ or even more to obtain shorter peptide core epitope of 3- to 6 aminoacid residues, which can be used for the recognition of the peptides according to the invention.

X₄X₃X₂X₁ and Y₁Y₂Y₃Y₄ are N-terminal or C-terminal additional amino acid residues, respectively so that the length of the peptide is preferably 12 or less, additional amino acid residues and their variants can be added from previous (prey. pre) and post specifications of

The consensus formulas of present invention can be transferred to this type of formula by replacing residues of C₁C₂C₃C₄C₄C₅C₆C₇ by the specific amino acid residues and their variants.

### Length of Preferred Epitopes of Antigen Peptides

“Short Epitopes” of about 5-13 Amino Acid Residues

“Very short epitopes” of 3-8 or about 5 amino acid residues. Prior art has studied long peptides covering usually 10-20 amino acid residues. The present invention is directed to peptide epitopes exposed on the viral surface. The epitopes are selected to direct immune reactions to conserved linear epitopes. The epitopes are relatively short about 5 amino acid residues long, preferably 3 to 8 amino acid residues, more preferably 4 to 7 aminoacid residues, most preferably 5 to 6 amino acid residues long. The invention reveals that a very short epitope can be enough for recognition by antibodies. The present invention also reveals specific novel conformational peptide epitopes, wherein the most important peptide part is only a few even 3 amino acid residues. The invention is further directed to the peptides of specific regions (A, B and C) in the large sialoside binding site of influenza virus hemagglutinin, wherein the short peptides comprise specific very short epitopes of at least three amino acid residues, preferably 3 amino acid residues of peptides 1-3. It is realized that the peptides mutate but these can be recognized as peptides according to the invention from the specific structures of very short peptide epitopes.

### Preferred Short Peptides of 5-13 or 5-12 Amino Acid Residue

In a preferred embodiment the invention is directed to specific peptides which have useful conformation for recognition by antibodies comprising at least 5 amino acid residues, more preferably at least 6 amino acid residues. The peptides do not have typical length of over 13 amino acid residues for recognition as T-cell peptides (regular influenza peptide vaccines comprise 16 or 20 meric or larger hemagglutinin peptides). The preferred length of the peptides are thus 5-13, more preferably 5-12 or 6-12 amino acid residues. The preferred optimal influenza surface peptides have lengths of 6-11, more preferably 6-10, or even more preferably 7-10 amino acid residues to include effective binding and conformation epitopes but omitting redundant residues.

The invention is in a preferred embodiment directed to conformational epitopes presented on hemagglutinin surface in the large sialoside binding site, as it is realized that antibodies against these cause effective blocking of the infection. The invention is directed to the use for immunizations of preferably conformational epitopes which can elicit immune responses by leukocytes, especially lymphocytes and most preferably B-cells.

Additional residues to improve presentation. The very short peptide epitope of about 3-8 amino acid residues long sequence preferred amino acid epitopes may be further linked to assisting structures. The preferred assisting structures includes amino acid residues elongating the short epitope by residues giving additional binding strength and/or improving the natural type presentation of the short epitopes. Additional residues may be included at amino terminal and/or carboxy terminal side of the short epitopes. Preferably there are 1-7, additional residues on either or 1-3 both side of the very short epitopes, more preferably 2-4 additional residues. The additional residues are represented, e.g., in Tables 6-9 as prev/pre and past residues or as first residues of following post peptide.

Conformational structures. The preferred short epitopes and/additional residues may further include conformational structures to improve the three dimensional presentation of the short epitope. The preferred conformational structures includes
A) conformational conjugation structures, such as a chemical linker structure improving the conformation of the peptides
B) single amino acid residue presentation improvement, which preferably includes replacement of non-accessible single residue, with a non-affecting structure such as linkage to a carrier or replacement by alanine or glycine residue.

The conformational structures include natural 3D analogues of the epitopes on the viral surfaces:

1) disulfide bridge mimicking structures, which may include natural disulfide bridges or chemical linkages linking cysteine residues to carrier
2) bridging structures including bridging structures

forming a loop for natural type representation

bridging between two peptide epitopes

The preferred peptide epitopes according to the invention comprise

a) a conformational peptide epitope comprising at least one cysteine residue or cysteine analogous amino acid residue conjugated from the side chain, and the peptide epitope comprises less than 100 amino acid residues, preferably less than 30 amino acid residues present in a natural influenza virus peptide and/or
b) the peptide epitope is a short peptide epitope comprising 3 to 12 amino acid residues, preferably comprising less than 12 amino acid residues, more preferably less than 11 amino acid residue.

In a preferred embodiment the peptide epitope is a conformational peptide epitope and a short peptide epitope.

Preferred conformational peptide epitopes include:

i) peptide 1 or peptide 2, which is conjugated from a cysteine or cysteine analogous residue side chain of the peptide epitope or
ii) peptide 3, which is in a cyclic form via a bridge formed by adding cysteine residues or cysteine analogous residues to the peptide sequence to form a loop comprising conformation similar to peptide loop on the surface of hemagglutinin protein.

More preferably, the peptide 1 is conjugated from a residue corresponding to cysteine 97 or

peptide 2 is conjugated from a residue corresponding to cysteine 139 as defined by the amino acid sequence of X31-hemagglutinin.

The preferred peptide 3 epitope comprises a cyclic or loop conformation of peptide 3, preferably a peptide of seven amino acid residue is cyclized by adding cysteine residues or cysteine analogous residues to N- and C-terminus of the peptides and forming a disulfide bridge or disulfide bridge analogous structure. Preferably, the cyclic or loop conformation has conformation similar to the conformation of peptide 3 on the surface of influenza virus hemagglutinin.

### Conjugates

It is realized that it is useful and preferred to represent the peptide epitopes according to the invention in a assay and/or binding method as a conjugated form. The background describes passive absorption of peptides but the present invention reveals very effective and robust assay, when the peptides are specifically conjugated covalently or by strong non-covalent linkage. The invention is further directed to specifically conjugated or covalently conjugated conformational epitopes represented for the immune system. In a preferred embodiment the invention is directed to conjugated structure, wherein the peptide is conjugated from the N-terminal or C-terminal end of the peptide sequence. In another preferred embodiment the peptide is conjugated only from N-terminal end, the invention revealed that such peptides can be effectively recognized by antibodies. In yet another preferred embodiment the peptide is conjugated from both N-terminal and C-terminal and to solid phase or soluble carrier.

In a preferred embodiment the peptide/peptide epitope according to the invention is separated from the carrier or solid phase by a linking atom group and/or linking atom group and a spacer. It is realized that the carrier or solid phase may affect the conformation of the conformational peptide. It is further realized that to long spacer structure would restrict the possibilities for the effective recognition of the peptides.

The invention is especially directed to representation of the conformational cyclic peptide with a flexible and inert spacer comprising a chain of one to five flexible atom structures connected with multiple single bonds such methylene (—CH₂—) groups, ether/oxy groups (—O—) or secondary amine group so that the spacer comprises at least one methylene group (—CH₂—) and more preferably at least two methylene, and even more preferably at least three methylenmethylene groups, the spacer comprise preferably not more than two and more preferably one or no rigid atom structures such as a double bond between carbon residues or an amide bond. In a preferred embodiment the spacer is an aminoalkanoic acid, preferably 2-8 carbon aminoalkanoic acid, more preferably 3-7 carbon aminoalcanoic acid and even more preferably 4-6 amino alkanoic acid such as aminohexanoic acid (amino caproic acid).

When the non-covalent linking structure is biotin, the biotin residue is considered totally being part of the linking structure, and the present invention is preferably directed to conjugating the biotin to the peptide by a flexible spacer, in a preferred embodiment the spacer is alkyl-chain in a preferred aminoalcanoic acid.

The invention is further directed to polyvalent presentation of the peptides according to the invention preferably conformational peptides according to the invention. It is realized that polyvalent presentation is especially useful when the peptides are aimed for inducing lymphocyte, especially B-cell meditated immune reactions/responses. especially for antibody production.

### Polyvalent Conjugates

The present invention is further directed to influenza binding directed analysis or therapeutic substance according to the formula PO

[PEP-(y)ₚ-(S)_{q}-(z)ᵣ-]ₙPO (SP1)

wherein PO is an oligomeric or polymeric carrier structure, PEP is the peptide epitope sequence according to the invention, PO is preferably selected from the group: a) solid phases, b) immunogenic and or oligomeric or polymeric carrier such as multiple antigen presenting (MAP) constructs, proteins such as KLH (keyhole limpet hemocyanin oligosaccharide or polysaccharide structure,
n is an integer≧1 indicating the number of PEP groups covalently attached to the carrier PO, S is a spacer group, p, q and r are each 0 or 1, whereby at least one of p and r is different from 0, y and z are linking groups, at least one of y and z being a linking atom group also referred as “chemoselective ligation group”, in a preferred embodiment comprising at least one an O-hydroxylamine residue —O—NH— or —O—N═, with the nitrogen atom being linked to the OS and/or PO structure, respectively, and the other y and z, if present, is a chemo selective ligation group, with the proviso that when n is 1, the carrier structure is a monovalent immunogenic carrier. In a preferred embodiment linking atom group z is biotin or equivalent ligand capable of specific strong non-covalent interaction.

In a preferred embodiment the conjugate comprises additional y2 or y2 and y3 groups forming additional linkages from N- or C-terminus or middle cysteine position to PEP to enhance the presentation of the conformational peptide group.

### Chemoselective Ligation Groups

The chemoselective ligation group y and/or z is a chemical group allowing coupling of the PEP-group to a spacer group or a PEP-(y)ₚ-(S)_{q}-(z)ᵣ-group to the PO carrier, specifically without using protecting groups or catalytic or activator reagents in the coupling reaction. According to the invention, at least one of these groups y and z is a O-hydroxylamine residue —O—NH— or —O—N═. Examples of other chemoselective ligation groups which may be present include the hydrazino group—N—NH— or —N—NR₁—, the ester group C(═O)—O—, the keto group C(═O)—, the amide group C(═O)—NH—, —O—, —S—, —NH—, —NR₁—, etc., wherein R₁ is H or a lower alkyl group, preferably containing up to 6 carbon atoms, etc. A preferred chemoselective ligation group is the ester group C(═O)—O— formed with a hydroxy group, and the amide group C(═O)—NH— formed with an amine group on the PO or Bio group, respectively. In a preferred embodiment, y is an O-hydroxylamine residue and z is an ester linkage. Preferably p, q, and r are 1. If q is 0, then preferably one of p and r is 0.

Preferred polysaccharide or oligosaccharide backbone (PO) structures include glycosaminoglycans such as chondroitin, chondroitin sulphate, dermantan sulphate, poly-N-acetylactosamine or keratan sulphate, hyaluronic acid, heparin, and heparin precursors including N-acetylheparosan and heparan sulphate; chitin, chitosan, starch and starch or glycogen fractions and immunoactivating glucose polysaccharides (e.g. pullulan type polysaccharides or beta-glucans such as available from yeast) or mannose (such as mannans) polysaccharides and derivatives thereof. A preferred backbone structure is a cyclodextrin. Useful starch fractions includes amylose and amylopectin fractions. The invention is specifically directed to use of water soluble forms of the backbone structures such as very low molecular weight chitosan polysaccharide mixture or c and on the other hand non-soluble or less soluble large polysaccharide especially for large polyvalent presentation especially for vaccines and immunizations.

Preferred spacer structure includes ones described for hydrophilic linker above, aminooxyacetic acid. According to an embodiment of the invention the spacer group, when present, is preferably selected from a straight or branched alkylene group with 1 to 10, preferably 1 to 6 carbon atoms, or a straight or branched alkenylene or alkynylene group with 2 to 10, or 2 to 6 carbon atoms. Preferably such group is a methylene or ethylene group. In the spacer group one or more of the chain members can be replaced by NH—, —O—, —S—, —S—S—, ═N—O—, an amide group —C(O)—NH— or —NH—C(O)—, an ester group —C(O)O— or —O—C(O)—, or —CHR₂, where R₂ is an alkyl or alkoxy group of 1 to 6, preferably 1 to 3 carbon atoms, or —COOH. Preferably a group replacing a chain member is —NH—, —O—, an amide or an ester group.

### Hydrophilic Spacer

The invention shows that reducing a monosaccharide residue belonging to the binding epitope may partially modify the binding. It was further realized that a reduced monosaccharide can be used as a hydrophilic spacer to link a receptor epitope and a polyvalent presentation structure. According to the invention it is preferred to link the peptide PEP via a hydrophilic spacer to a polyvalent or multivalent carrier molecule to form a polyvalent or oligovalent/multivalent structure. All polyvalent (comprising more than 10 peptide residues, preferably more than 100 and for vaccination even more that 1000 up to 100 000 or million or 10 000 000 million or more in large polyvalent conjugates) and oligovalent/multivalent structures (comprising 2-10 peptide residues) are referred here as polyvalent structures, though depending on the application oligovalent/multivalent constructs can be more preferred than larger polyvalent structures or vice versa. The hydrophilic spacer group comprises preferably at least one hydroxyl group or alkoxy/ether group. More preferably the spacer comprises at least two hydroxyl groups and most preferably the spacer comprises at least three hydroxyl groups.

According to the invention it is preferred to use polyvalent conjugates in which the hydrophilic spacer group linking the peptide sequences to polyvalent presentation structure is preferably a flexible chain comprising one or several —CHOH— groups and/or an amide side chain such as an acetamido —NHCOCH₃ or an alkylamido. The hydroxyl groups and/or the acetamido group also protects the spacer from enzymatic hydrolysis in vivo. The term flexible means that the spacer comprises flexible bonds and do not form a ring structure without flexibility. A reduced monosaccharide residues such as ones formed by reductive amination in the present invention are examples of flexible hydrophilic spacers. The flexible hydrophilic spacer is optimal for avoiding non-specific binding of neoglyco lipid or polyvalent conjugates. This is essential optimal activity in bioassays and for bioactivity of pharmaceuticals or functional foods, for example.

A general formula for a conjugate with a flexible hydrophilic linker has the following Formula HL:

[PEP-(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z

wherein L₁ and L₂ are linking groups comprising independently oxygen, nitrogen, sulphur or carbon linkage atom or two linking atoms of the group forming linkages such as —O—, —S—, —CH₂—, —NH—, —N(COCH3)—, amide groups —CO—NH— or —NH—CO— or N═N-(hydrazine derivative) or hydroxylamine —O—NH— and NH—O—. L1 is linkage from hydrophilic spacer to additional spacer X or when n=0, L1 links directly from N- or C-terminus or middle cysteine position to PEP.

p1, p2, p3, and p4 are independently integers from 0-7, with the proviso that at least one of p1, p2, p3, and p4 is at least 1. CH₁₋₂OH in the branching term {CH₁₋₂OH}ₚ₁ means that the chain terminating group is CH₂OH and when the p1 is more than 1 there is secondary alcohol groups —CHOH— linking the terminating group to the rest of the spacer. R is preferably acetyl group (—COCH₃) or R is an alternative linkage to Z and then L₂ is one or two atom chain terminating group, in another embodiment R is an analog forming group comprising C₁₋₄ acyl group (preferably hydrophilic such as hydroxy alkyl) comprising amido structure or H or C₁₋₄ alkyl forming an amine. And m>1 and Z is polyvalent carrier. PEP is peptide according to the invention, X is additional spacer such as spacer S in formula PO.

### Preferred Novel Peptides and Peptide Compositions

The invention is further directed to peptides 1-3 and short and/or conformational forms thereof as antigenic peptide or peptide composition comprising at least one peptide, preferably peptide 2 or peptide 3.

The peptides 2 and 3 were observed to be targets of especially effective immune responses, specifically antibody responses. The preferred peptide 2 and 3 three includes H1, H3, and H5 peptides, more preferably H1 and H3, and conformational and/or short peptide, more preferably human infecting variants of the peptides.

In another preferred embodiment, the antigenic peptide composition comprises at least two peptides selected from the group peptide 1, peptide 2 and peptide 3, and in another embodiment all three peptides peptide 1, peptide 2 and peptide 3, and in a preferred embodiment both of the highly immunogenic peptides peptide 2 and 3.

### Methods for Binding and Selection of Molecules, Especially Antibodies Against the Peptides

### Influenza Antibody Target Peptides

The invention revealed specific peptides which are located on surface of influenza virus divalent sialoside binding site. The peptides can be recognized by antibodies, which then can block the binding to the large binding site also referred as divalent sialoside binding site on the surface of influenza virus. The peptides are thus targets for antibody recognition methods and antibody selection methods based on the specific recognition of the peptides by antibodies.

The antibody recognition method measures of binding of one or more antibody to the peptides. The antibody selection method further involves selection of the binding antibodies, which have desired binding affinity.

### Antibody Fragments, Peptides and Equivalent Binding Reagents

It is further realized that multiple other binding reagents equivalent of antibodies or modulator molecules can be selected similarly as antibodies. In a preferred embodiment the other binding reagents are proteins with varying structures like antibodies, antibody fragments or peptides or part of repetitive oligomeric or polymeric structure resembling peptides such as peptide mimetics, which are well known in the art, or nucleic acid derived binding molecules with repetitive structure such as aptamers or a molecule derived from a molecular library comprising molecules large enough for binding.

It is realized that production of a molecular library for screening of binding reagents against the peptides according to the invention is a routine process known for skilled person and when the molecular library is large enough the finding of suitable other binding reagents is feasible.

It is further realized that the binding reagents have inherently common chemical structures corresponding to the three dimensional structures represented by the peptides on the influenza hemagglutinin surfaces. The peptides according to the invention are naturally located on protein surface and thus comprise at least one amino acid residue comprising polar side chain, more preferably at least two, even more preferably at least three polar side chains. The preferred binding structures recognizing the peptide by hydrogen bond or ionic interactions further includes at least one, more preferably at least two and most preferably at least three polar functional group such as a hydroxyl group, carboxy group including keto group, carboxylic acid group, or aldehyde group, amine group or oxygen linked to phosphorus or sulphur atom such as in sulphate, sulfonyl or phosphate structures or polar halogen atoms such as fluoro-, chloro- or bromo-halogens, more preferably fluoro or chloro-linked to carbon atoms. The invention is directed to the recognition of hemagglutinin peptides by a reagent comprising at least the same amount of polar structures as represented by the desired target hemagglutinin peptide. The invention is further directed to the recognitions of non-polar structures included in the peptide structures by non-polar structures such as non-polar amino acids or amino acid mimetics on the binding reagents.

### Antibody Selection Methods

It is realized that antibodies can be selected in numerous ways involving the step of binding of antibody to the peptide and selection of antibodies binding to the target peptides with desired binding affinity. The preferred binding and/or selection methods include contacting the peptide with a library or multitude of proteins being antibody production involved proteins such as antibodies or molecules representing peptides antibodies. Preferably, the contacting occurs on the surface of genetic entities, such as cells bacteria, or phages, viruses or alike, capable of representing a variant of antibody production involved proteins. In a preferred embodiment genetic entities include immune cells such as leukocytes, preferably lymphocytes, representing antibodies or phages or bacteria representing antibodies or in another embodiment preferred genetic entities include immune cells such as leukocytes, preferably lymphocytes, representing T-cell receptors and/or HLA antigens

The invention is especially directed to the representation of the peptides in libraries of antibodies or antibody fragments for activation of immune cells by the peptides, or in phage display libraries to observe binding of strongly binding antibodies.

The peptides were selected based on the location on the virus surface. It is realized that immunization or selection of antibodies with different longer peptides would produce immune reactions against structures outside of the binding site of the antibodies.

The methods of binding to influenza virus peptides according to the invention, wherein the method is used for selection of chemical entities, preferably antibodies, preferably from a library of the entities and the selection is performed in vivo, ex vivo or in vitro and optionally the detection is observing the result of the selection.

The preferred method involves specific conjugation of the peptide to matrix by a covalent bond or strong non-covalent interaction.

The covalent bond is preferably formed from sulphur atom of a cysteine residue, preferably to maleimide or analogous structure or to a sulphur of cysteine in the matrix or the strong non-covalent interaction is binding of a ligand to a protein, preferably biotin binding to an avidin protein and preferably the peptide is biotinylated.

The binding and/or selection method is in a preferred embodiment an in vitro immunoassay or in vitro selection of an antibody library such as phage display antibody library, preferably involving extensive washing.

In another embodiment the method is an ex vivo or in vivo immunization method, preferably involving activation of immune cells, more preferably lymphocytes, most preferably B-cells.

### Search and Evaluation of Potentially Autoimmunogenetic Peptides Form Databases and Protein Conformations

In a preferred embodiment the binding and/or selection method involves a step of searching any of the peptide epitopes 1-3 of an hemagglutinin from database comprising human genome coded peptide sequences and selection of peptides, which are not expected to cause immune reaction against a human (or animal) subject.

In the preferred search method, when similar peptide sequence(s) is (are) found from human (or animal) genome sequence, these will be evaluated with regard to

i) availability for human (animal) immune system with regard to presence of the peptide sequence on surface of a protein and/or on a cell surface protein and preferably selecting peptides which are not available for human (animal) immune system and/or
ii) conformation of the peptide in a human (or animal) protein being similar to conformation of the peptide on the hemagglutinin surface and preferably selecting peptides which do not have similar conformations on human proteins.

Recognition by immune system. The peptides are recognizable by the immune system of the patient and can induce immune reaction against the peptides. The immune reaction such as an antibody reaction and/or cell mediated immune reaction can recognize the peptide epitope on the surface of the virus and diminish or reduces its activity in causing disease. In a preferred embodiment the invention is specifically directed to peptides recognized by antibodies of a patient and development of such peptides to vaccines.

Preferred immune recognition by relevant species such as human and/or pandemic animal species. It is realized that most of the prior art has studied the immunoreactivity of various, in general long, peptide epitopes with regard to species used for immunological experiments such as mice, rats, rabbits or guinea pigs. It is realized that studies with regard to these immune systems is not relevant with regard to the human disease and there is multitude of results supporting this fact. The results have been very varying and does not reveal useful short epitopes with regard to human immune system.

The present invention is directed to analysis of the effect of the antigen peptides in animal species from which influenza infection is known to effectively spread to humans (see U.S. patent application No. 20050002954). Preferred animal species are avian species and/or pig. The preferred avian species includes poultry animals such as chicken and ducks, and wild bird species such as ducks, swans and other migratory water birds spreading influenza virus.

The present invention revealed that the short peptide epitopes are useful against viruses spreading from the relevant species to human patients. It was realized that the epitopes are recognizable on the surfaces of viruses and antibodies binding to peptides would block the carbohydrate binding sites of the viruses.

Screening of antibodies. The invention is directed to screening methods to reveal natural antibodies binding to peptides, preferably peptides derived from carbohydrate binding sites of human pathogens especially carbohydrate binding sites of parthogens comprising large carbohydrate binding sites involving binding to multiple monosaccharide units, more preferably including binding sites for two sialic acid structures. Preferably the invention is directed to screening of human natural antibody sequences against peptides derived from viruses or bacteria, more preferably against carbohydrate binding sites of influenza viruses.

It is realized that antibodies may be screened by affinity methods involving binding of antibodies to the peptide epitopes. The peptide epitopes may be conjugated to solid phase for the screening, preferably for screening of human antibodies. In a preferred embodiment the peptides are screened from blood, blood cells or blood derivative such as plasma or serum of a patient. In another embodiment the antibodies are screened from a phage display library derived from blood cells of a patient or several patients or normal subjects, preferably expected to have immune reaction and antibodies against the peptides disclosed in the invention.

Screening of peptides. The invention is further directed to screening of the preferred peptide epitopes and analogous peptides and conjugates thereof against human immune reactions for development of the optimal vaccines and antibody development products.

The invention is further directed to further screening of, and binding analysis of peptides, which are recognized by patients immune system preferably by natural antibodies of a patient. The invention is directed to screening methods to reveal further peptides derived from carbohydrate binding proteins (adhesions/lectins) of human pathogens, especially carbohydrate binding sites of parthogens comprising large carbohydrate binding sites involving binding to multiple monosaccharide units, more preferably including binding sites for two sialic acid structures.

Preferred types of influenza viruses. The influenza viruses are preferably viruses involving risk for human infection, including human influenza viruses, and/or potentially human infecting pandemic influenza viruses such as avian influenza viruses. More specifically the preferred virus is influenza A, influenza B and influenza C viruses, even more preferably influenza A or B, and most preferably influenza A. Preferably influenza A is a strain infecting or potentially infecting humans such as strains containing hemagglutinin type H1, H2, H3, H4, or H5.

### Preferred Peptides or Groups of Peptides for Influenza Viruses

The invention is directed to specific peptide epitopes and variants thereof for treatment of influenza (including prophylactic or preventive treatments). The invention is specifically directed to specific peptide epitopes and groups thereof for treatment of specific subtypes of influenza such as influenzas involving hemagglutinin types H1, H2, H3, H4, or H5, more preferably H1, H2, H3, or H5, even more preferably H1, H3 or H5. Especially human infecting types of hemagglutinins, especially hemagglutinins H1, H3, and H5 viruses are preferred and even more preferably H1 and H3 are preferred. In an especially preferred embodiment the peptide is conformational peptide 2 and 3, even more preferably peptide 3, from the preferred hemagglutinin types including H1, H3 and H5, H1 and H3 and most preferably H3.

### Several Peptides Agains the Same Hemaglutin or Homologous Hemagglutinins

It is realized that part of the sequences comprise relatively fast mutating semiconserved residues. Production of peptides with multiple variants for longer about 20 meric peptides is chemically feasible by standard technologies, see for example influenza patent applications of Variation biotechnology and related background publications. The shorter peptide epitopes according to the present invention are even more effective for synthesis and includes less variants. In a preferred embodiment the peptide composition for binding and selection methods or according to the invention includes variants of the peptides currently present in influenza virus. The preferred and most relevant variants includes 1-5 variants for peptides 1-3, more preferably 1-3 variants or 2 or 3 variants. The amount of variants needed depend on the current status of evolution of the specific peptide, when the peptide is changing from one major variant to another there is multiple variants present typically at least one major old variant e.g. WVR variant of H3 and more recent RVR variants of peptide 3 were present simultaneously, see tables 9. It is further realized that the peptide 2 comprises especially many semiconserved residues and invention is directed to including more variants typically two to five, more preferably 2-4 variants or most preferably at least 2 or 3 variants of it for effective vaccine. Less important residues at N- or C-terminus may be more varying such as N-terminal residue of peptide 3. In case a linear peptide or a conformational peptide would be considered, preferably by analysis from databases, as autoimmunity causing non-autoimmunogenice variants thereof are selected and/or peptide(s) from another region(s) (peptide 1 or peptide 2 or peptide 3) are included in the vaccine.

The invention is directed to preferred peptide compositions for binding analysis and/or peptide selection, and especially immunization and/vaccination, when the composition comprises at least 2, preferably 2-5, more preferably 2-4, different peptide sequences, preferably conformational sequences according to the invention, which are variants of the same peptide (selected from the group peptide 1, peptide 2 and peptide 3, more preferably peptide 2 and 3). The preferred vaccine composition preferably further comprises a second type of immunogenic peptide, and optionally current variant(s) thereof, from influenza selected from the group:
i) a peptide from different region of hemagglutinin, selected from the group peptide 1, peptide 2 and peptide 3,
ii) a peptide from the same region of hemagglutinin but from different hemagglutinin type (preferably from hemagglutinins H1-H5, more preferably from the preferred hemagglutinins according to the invention and
iii) another known antigenic peptide from
   a. another site of hemagglutin protein such as the known peptide vaccine epitopes conserved at cleavage site of precursor HA0 from hemagglutinin or other longer hemagglutinin peptides
   b. another protein of influenza virus, preferably a conserved
      i. peptide epitopes of M2 protein
      ii. peptide epitopes of NP protein of influenza

In yet another preferred embodiment the vaccine composition comprises at least two variants of two peptides according to i), preferably peptide 2 and peptide 3 and in yeast another preferred embodiment at least additional peptide according to ii) and more preferably at least two peptides according to ii) and most preferably at least one, more preferably at least two variants there of.

It is further realized that relatively good influenza restricting or taming though not effectively blocking responses have been obtained by M2 peptides of influenza, or by HA₀ or NP protein based epitopes and the vaccines and known combinations therefore it would be beneficial to combine with current peptides according to the invention.

In a preferred embodiment the preferred compositions for the methods according to the invention comprises peptide 2 and 3 of two (preferably H1 and H3) or three hemoglutinins (preferably H1, H3 and H5). In specifically preferred embodiment preferably H1 and H3 hemagglutinin and at least one variant of one peptide, more preferably at least one variant of two peptides, and in another preferred embodiment at least one variant of three or all four peptides, and it is especially preferred to include variants of peptide 2, even 3 or more variants, and optionally a least one variant of one peptide 3 preferably H3 type of peptide 3 for vaccination or analysis of current influenza

The invention is further directed to combinations of current peptides with complete hemagglutinin protein or another influenza virus protein or domain there of comprising e.g. about 50-100 aminoacid residues, known as potential influenza vaccines and or open influenza viruses or analogous viral particles comprising surface protein(s) of influenza.

The preferred HA0 from hemagglutinin peptides includes e.g. ones developed by Merck and Biondvax and known in background of their publications. Other preferred hemagglutinin peptides from includes e.g. ones developed by Variation biotechnology e.g. including peptide 1 and peptide 4 described in WO06128294 (Dec. 7, 2006). and Biondvax including peptide HA91 (e.g. WO07066334, Jun. 14, 2007) directed to longer peptides epitopes which are not conformational and conjugated according to the present invention.

The preferred M2 protein or peptide epitopes are developed by the companies including Merck US (peptides), Acambis (with Flanders Univ.), AlphaVax (with NIH, pandemic), VaxInnate (with Yale Univ.), Dynavax (with support from NIH), Cytos Biotech, CH), GenVec (with NIAID), or Molecular Express, Ligocyte or Globe immune or Biondvax (Israel, Ruth Amon and colleagues) and known from the background of their publications. M2 also referred as M2e is common (conserved) antigen and ion channel on influenza, it is not accessible on viral surface but targeted on infected cells (assembly of virus) and it does not cure effectively but relieve disease (Science 2006, Kaiser).

The preferred NP protein (nucleoprotein of influenza) or peptide epitope are developed e.g. by the companies Biondvax, AlphaVax, GenVec and known from the background of their publications)

### Preferred Conserved Amino Acid Epitopes, Antigen Peptides, for Vaccine or Antibody Development

The present invention is preferably directed to following peptide epitopes, and any linear tripeptides or tetrapeptides derivable thereof or combinations thereof for vaccine and antibody development, preferably directed for the treatment of human influenza. The invention is further directed to elongated versions of the peptides containing 1-3 amino acid residues at N- and/or C-terminus of the peptide. The numbering of the peptides is based on the X31-hemagglutinin if not otherwise indicated. This indicated corresponding position of the peptides in three dimensional structure of the hemagglutinin and same position with regard to conserved cysteine bridge for Peptide 1 and Peptide 2 and presence in the loop structure as described for Peptide 3.

The invention is specifically directed to sequencing and analysing corresponding peptides from new influenza strains, because the viruses have tendency to mutate to avoid human immune system. The invention further revealed that it is possible to use several peptides according to the invention. Persons resistant to influenza virus had antibodies against 2 or 3 peptides. The invention is directed to vaccines against single type of influenza H1, H2, H3, H4 or H5.

The invention is further directed to peptide compositions comprising at least one peptide, more preferably at least two and most preferably at least three peptide, against at least two, more preferably at least three, different hemagglutinin subtypes, preferably against H1, H3, and/or H5. In a specific embodiment the invention is directed to peptides of H5-hemagglutinins aimed for treatment or prevention of avian influenza.

It is further realized that similar peptides may be derived from other influenza virus hemagglutinins. The invention is specifically directed to defining structurally same peptide positions from influenza B, Influenza C and other hemaglutinin subtypes such as H6, H7, H8, or H9.

It is further realized that the peptides may be used in combination with known and published/patented peptide vaccines against influenza and/or other influenza drug. The invention is specifically directed to the use of the vaccines together with hemagglutinin binding inhibiting molecules according to the invention, preferably divalent sialosides. The invention is further directed to the use of the molecules together with neuraminidase inhibitor drugs against influenza such as Tamiflu of Roche or Zanamivir of GSK or Peramivir of Biocryst or second generation neuraminidase inhibitors such as divalent ones developed by Sankyo and Biota

The peptides are preferably aimed for use as conjugates as polyvalent and/or immunomodulator/adjuvant conjugates. The preferred epitopes do not comprise in a preferred embodiment additional, especially long amino acid sequences. The length of the short conformational epitopes is preferably less than 13 amino acid, and preferred shorter epitopes, as described for the short epitopes. There are preferably less than 7 amino acid, more preferably less than 5, more preferably less than 3 and most preferably less 1 or 0 additional amino acid residues, directly continuing from the original hemagglutinin sequence.

It is realized one or several of the amino acid residue can be replaced by mimicking residue having similar conformation. The invention is further directed to methods for optimization of the peptides so that part of the sequence, which is preferably analyzed by molecular modelling and/or binding method according to the invention, especially N- and/or C-terminal amino acid residue(s)/additional residues at N- or C-terminus, be changeable to similar residues supporting the conformation of the peptide. The invention is further directed to the optimization of chemical epitopes of the linear and or conformational peptides by standard peptide optimization methods, which in a preferred embodiment includes introduction of structures resistant to proteases and or peptidases present in the patient.

Many peptide vaccines have been described against influenza virus. These contain various peptides of the virus usually conjugated to carriers, or other immunogenic peptides and/or adjuvants and further including adjuvant molecules to increase antigenicity.

Person skilled in the art can determine the corresponding amino acid position from other influenza hemagglutinins in relation to most conserved amino acid residues and/or position of disulfide bridges and design similar peptides containing 1-3 different, more preferably 1-2 different amino acid residues, most preferably only one different amino acid residue. Design of analogs and elongated variants of the peptides involves analysis of the surface presentation of the peptides, so that these would be accessible for analytic/diagnostic and/or therapeutic recognition by specific binding agents, such as antibodies, peptides (such as phage display peptides), combinatorial chemistry libraries and/or aptamers.

### Preferred Hemagglutinin Peptides

Region of Amino Acid at Positions of about 210- to 230 of Hemagglutinin

Similarity is observed between influenza A viruses for example as partial, very short peptide epitope sequence KVR and iso forms in hemagglutinin type H1 sequences and similar positively charged RVR in current strains H3 after about year 2000, WVR in older H3 and KVN in H5. The region is favoured because presence on the surface of the virus available for immune recognition and because antibodies binding to the region would interfere with carbohydrate binding of the virus. The peptides form a conserved loop type epitope which can be further used for production of cyclic peptides. The invention is especially directed to conformational epitopes represented by the cyclic peptide structure.

It is realized that it is useful and preferred to represent the peptide 3 epitopes in a assay and/or binding method as a conjugated form. The background describes passive absorption of peptides but the present invention reveals very effective and robust assay, when the peptides are specifically conjugated covalently or by strong non-covalent linkage. The invention is further directed to specifically conjugated or covalently conjugated conformational epitopes represented for the immune system. In a preferred embodiment the invention is directed to conjugated structure, wherein the peptide is conjugated from the N-terminal or C-terminal end of the peptide sequence. In another preferred embodiment the peptide is conjugated only from N-terminal end, the invention revealed that such peptides can be effectively recognized by antibodies. In yet another preferred embodiment the peptide is conjugated from both N-terminal and C-terminal and to solid phase or soluble carrier.

In a preferred embodiment the cyclic peptide is separated from the carrier or solid phase by a linking atom group and/or linking atom group and a spacer.

### Preferred KVR-Region Peptides of H1 Similar Peptides

The conserved amino acid (from amino terminus to C-terminus) Lys222-Va1223-Arg224 KVR homologous to WVR-region of X31 hemagglutinin forms an excellent target for recognition of influenza virus. This relatively conserved sequence is present e.g. in the sequence RPKVRDQ of A/South Carolina/1/1918 (H1N1), also known as “Spanish Flu”-hemagglutinin. The peptide was modelled as an exposed sequence on the surface of the virus. The peptide sequence is preserved in hundreds human influenza A viruses. The region comprise a tripeptide Lys222-Va1223-Arg224 (KVR), which is a preferred peptide epitope according to the invention and present in longer peptide epitopes. Preferred peptide epitopes includes heptapeptide RPKVRDQ and further includes pentapeptides: RPKVR, PKVRD, KVRDQ and hexapaptides RPKVRD and PKVRDQ. The proline is preferred as an amino acid affecting the conformation of the peptide, the D-residues is preferred as a semi-conserved amino acid residue, it may be replaced by similar type amino acid residue

### Conserved Peptide 3 Region of Hemagglutinin 2, H2

The invention revealed that human hemagglutin 2 also contains conserved Peptide 1 region the examples of the sequences includes RPEVNGQ and RPKVNGL at position 99-105, see Table 8, the epitope comprises additional aminoacid residues K and E- especially at N-terminal side, with consensus sequence RPXVNG or PXVNG, RPXVN, RPXV, PXVN, XVNG, RPX, PXV, XVN wherein X is any aminoacid preferably E or K

### Preferred WVR-Region Peptides of H3 Similar Peptides

The conserved amino acid (from amino terminus to C-terminus) Trp222-Val223-Arg224 WVR of region B of X31 hemagglutinin forms another excellent target for recognition of influenza virus. The peptide was modelled as an exposed sequence on the surface of the virus. The peptide sequence is preserved in more than hundred human influenza A viruses. The region comprise a tripeptide Lys222-Val223-Arg224 (WVR), which is a preferred peptide epitope according to the invention and present in longer peptide epitopes. Preferred peptide epitopes includes heptapeptide RPWVRGL and further includes pentapeptides: elongated variants pentapeptides, RPWVR, PWVRG, WVRGL and hexapaptides RPWVRG and PWVRGL. The proline is preferred as an amino acid affecting the conformation of the peptide, the L-residues is preferred as a semi-conserved amino acid residue, it may be replaced by similar hydrophobic amino acid residue. The preferred variants include ones where W is replaced by R-residue.

### Preferred KVN-Region Peptides of H5 Similar Peptides

The conserved amino acids Lys222-Val223-Asn224 (KVN, from amino terminus to C-terminus) observable for example from H5-hemagglutinins A/Vietnam/1203/2004 (H₅N₁) or A/duck/Malaysia/F119-3/97 (H5N3), corresponding to conserved region B of X31 hemagglutinin forms a further target for recognition of influenza virus. The peptide was modelled as an exposed sequence on the surface of the virus. The peptide sequence is preserved in more than hundred human influenza A viruses.

Preferred peptide epitopes further includes elongated variants peptides being the heptapeptide RPKVNGQ, hexapeptides RPKVNG, and PKVNGQ, pentapeptides RPKVN, PKVNG, KVNGQ, RPKVNG, and PKVNGQ. The penta- to hepta peptides all includes the preferred tripeptide structure KVN. The invention is further directed to tetrapeptides RPKV, PKVN, including the preferred subepitope KV and KVNG and VNGQ including preferred subepitope VN. The proline is preferred as an amino acid affecting the conformation of the peptide, it may be replaced by similar type amino acid residue.

The invention is specifically directed to consensus of Peptide 3 region

### RPX₁VX₂X₃

### X₁ is K, E, R or W

### X2 is N, or R

X3 is noting, D or G.
Cyclic Peptides of the Region about 210-230

The invention is further directed cyclic peptides including the preferred peptide epitopes above. Most preferably a natural type heptapeptides RPKVRDQ, RPWVRGL, RPKVNGQ linked to a cyclic peptide by residues X and Y:

X-H7-Y,

wherein H7 is the heptapeptide and
X is group forming cyclic structure with group Y,

In a preferred embodiment X and Y are Cys-residues forming disulfide bridge With each other.

The groups X and Y include preferably pair of specifically reactive groups such as amino-oxy (—R—O—NH2) and reactive carbonyl such as aldehyde or ketone; azide (—R—N═NH2) and reactive carbonyl such as aldehyde or ketone

Region of Amino Acid at Positions of about 85- to about 100/98-106

Similarity is observed between influenza A viruses within a region corresponding to the amino acids located before cysteine 97 in the structure of H3 hemagglutinin X31. The region is favoured because presence on the surface of the virus available for immune recognition and because antibodies binding to the region would interfere with carbohydrate binding of the virus. The region is mainly semiconserved, there is similar variants of the sequences, which are relatively well conserved within each hemagglutinin type.

### Preferred TSNSENGT(C)-Region of H1 Type Viruses

The amino acid residues before the X31Cys97 equivalent are located e.g. at positions 86-93 of A/South Carolina/1/1918 (H1N1) with sequence TSNSENGT(C) or NSENGT(C). Especially the region TSESEN, more preferably SESEN is well exposed on the surface of the virus, while the conformation of the last two amino acid residues GT in the region are less well exposed. In a preferred embodiment one or both of the C-terminal residues and optionally also the Cys-residue are included as “additional residues” to achieve optimal presentation and/or conformation. Preferred variants includes peptides NPENGT(C), PNPENGT(C) and TPPENGT(C); NSENGI(C), PNSENGIC(C) and TPNSENGIC (C).

The preferred consensus sequence includes NX₁ENGX₂(C), and shorter variants ENGX₂(C), NX₁EN, wherein X₁ and X₂ are variable residues, preferably ones described above and cysteine (C) may be present or absent, preferably present, more preferably as thiol conjugate; and ENG.

### Conserved Peptide 1 Region of Hemagglutinin 2, H2

The invention revealed that human hemagglutin 2 also contains conserved Peptide 1 region the examples of the sequences includes NPRNGLC AND NPRYSLC at position 99-105, see Table 8, the epitope comprises additional aminoacid residues K and E-especially at N-terminal side, with consensus sequence NPR or NPRXXL(C), PRXXL(C), RXXL(C), wherein cysteine (C) may be present or absent, preferably present, more preferably as thiol conjugate;

### Preferred SKAFSN(C)-Region Peptides of H3 Type Viruses

The conserved aminoacid (from amino terminus to C-terminus) Ser9′-Lys92-Ala91-Phe94-Ser95-Asn96-Cys97 (SKAFSNC) as presented in human H3-hemagglutinin belong to, at least partially conserved, and exposed and available region. The peptide sequence is preserved in more than hundred human influenza A viruses H3. Preferred peptide epitopes further includes elongated varianta AFSN, SKAFSN, SKAFS, and SKAF. In a preferred embodiment one or both of the C-terminal residues and optionally also the Cys-residue are included as “additional residues” to achieve optimal presentation and/or conformation.

Recent A-influenza viruses contain especially preferred variants wherein F is replaced by Y(tyrosine): AYSN, SKAYSN, SKAYS, and SKAY. Furthermore variant wherein Lysin is replaced by T (theronine) are preferred: STAYSN, STAYS, and STAY, which are also present in recent influenza viruses.

### Preferred KXNPVNXL(C)-Region of H5 Type Viruses

The amino acid residues before the X31Cys97 equivalent are located e.g. at positions 99-106 of A/duck/Malaysia/F119-3/97 (H5N3) with sequence KDNPVNGL(C) and at positions of 98-105 of A/Viet Nam/1203/2004 (H5N1) with the sequence KANPVNDL(C). Especially the region KXNPVN, more preferably XNPVN is well exposed on the surface of the virus, while the conformation of the last two amino acid residues, XL, in the region are less well exposed. In a preferred embodiment one or both of the C-terminal residues and optionally also the Cys-residue are included as “additional residues” to achieve optimal presentation and/or conformation.

Region of Amino Acid at Positions of about 130- to about 140

Similarity is observed between influenza A viruses within a region corresponding to the amino acids located before cysteine 139 in the structure of H3 hemagglutinin X31, and in a preferred embodiment also including Cys139 equivalent and few following amino acid residues. The region is favoured because presence on the surface of the virus available for immune recognition and because antibodies binding to the region would interfere with carbohydrate binding of the virus. The region is mainly semiconserved, there is similar variants of the sequences, which are relatively well conserved within each hemagglutinin type.

### Preferred TTKGVTAA(C)-Region of H1 Type Viruses

The amino acid residues before the hemagglutinin X31-Cys139 equivalent are located e.g. at positions 132-139 of A/South Carolina/1/1918 (H1N1) with sequence TTKGVTAA(C). The preferred exposed sequence includes the Cys residue and 1-4 amino acid residues after it. In a preferred embodiment one or two additional residues of the C-terminal and/or N-terminal residues and optionally also the Cys-residue are included as “additional residues” to achieve optimal presentation and/or conformation.

The H1 Peptide 2 is preferred at position 148-153 in sequences containing signal sequence see Table 6, see Table 8, the Table describes additional aminoacids TK, TN, and TR at aminoterminal side and preferred additional sequences as Peptide 2b and its N-terminal aminoacids and di- to tetrapeptides, the preferred core epitopes are

### GVTAA(C) and GVTAS(C), and

### VTAA(C) and VTAS(C),

### VTAX(C),

cysteine (C) may be present or absent, preferably present, more preferably as thiol conjugate.

### Conserved Peptide 2 Region of Hemagglutinin 2, H2

The invention revealed that human hemagglutin 2 also contains conserved Peptide 1 region the examples of the sequences includes SQGCAV AND SWACAV, see Table 8, the epitope comprises additional aminoacid residues at N-terminal side, preferably TTGG, or TGG, OR GG, with consensus sequence TTGGSXXCAV or

### GSXX(C)AV

### GSX₁X₂(C)A

GSX₁X₂(C), wherein
XᵢX₂ are any aminoacid preferably X₁ is Q and W; and X₂ is A or G, respectively cysteine (C) may be present or absent, preferably present, more preferably as thiol conjugate, when C is absent in the middle of chain it is replaced by glycine or alanine preferably by glycine.

### Preferred GGSNA-Region Peptides of H3 Type Viruses

The conserved amino acid (from amino terminus to C-terminus) Gly134-Gly135-Ser136-Asn137-Ala138 of region A (GGSNA) of region B of X31 hemagglutinin forms an excellent target for recognition of influenza virus. The peptide was modelled as an exposed sequence on the surface of the virus. The peptide sequence is preserved in more than hundred human influenza A viruses.

Preferred peptide epitopes further includes elongated variants such as GGSNACKRG, GSNACKRG, SNACKRG, NACKRG, GGSNACKR, GSNACKR, SNACKR, NACKR. The preferred variants includes sequences wherein N is replaced by S, or T and other variants of recent influenza viruses with 1-2 substitutions, especially aromatic aminoacid variants including tyrosine.

Other preferred sequences includes SYACKR and SSACKR and N- and C-terminally elongated variants with additional 1-3 amino acids the consensus sequences

### SX₂A(C)KR

### X₁ SX₂(C)KR

### GX₁SX₂A(C)KR

### SX₂A(C)K

### X₁SX₂(C)K

### GX₁SX₂A(C)K

### SX₂A(C)

### X₁SX₂(C)

### GX₁SX₂A(C)

Wherein X₁ is any aminoacid preferably G, T, or E

And X2 is any amino acid preferably N, Y or S,
cysteine (C) may be present or absent, preferably present, more preferably as thiol conjugate, when C is absent in the middle of chain it is replaced by glycine or alanine preferably by glycine.

### Preferred DASSGVSSA(C)PY-Region of H5 Type Viruses

The amino acid residues before the hemagglutinin X31-Cys139 equivalent are located e.g. at positions 142-150 DASSGVSSA(C)PYNG (numbering including signal peptide) of A/duck/Malaysia/F119-3/97 (H₅N₃) and at positions of 142-150 of A/Viet Nam/1203/2004 (H5N1) with the sequence EASLGVSSA(C)PYQG. Especially the region (E/D)ASXGVSSA, more preferably GVSSA is well exposed on the surface of the virus. In a preferred embodiment one or both of the C-terminal residues and optionally also the Cys-residue are included as “additional residues” to achieve optimal presentation and/or conformation.

### Less-Available but Conserved Sequences

The invention reveal novel peptide epitopes, which are very conserved among influenza viruses, but less surface exposed and thus less available regular immunotherapies on cell surfaces. It is realized that presence of such peptides for example on T-cell receptors or antibodies against these are indicative of immune reaction against influenza. Studies of such immune reactions are useful for analysis of immune reactions against influenza, though such reaction may be less useful against influenza. Immune reactions are indications about the strength and direction of immune response. The analysis may be used peptide analysis of presence of influenza or other influenza diagnostics. The sequences are further useful for PCR analysis of the infection by analysis of nucleic acid sequences corresponding to the conserved peptide epitopes.

### Conserved Less-Available “Core Sequences” of Influenza a Viruses

Beside the active surface sequences the present invention revealed certain other conserved amino acid sequences present in the viruses. The less available sequences referred here as “core sequences” comprise usually large hydrophobic amino acids. Most of the sequences are conserved in larger groups of influenza viruses such as influenza A or influenza B viruses. The invention is especially directed to the analysis of the highly conserved core sequence(s) together with one or several of the antigen peptides, which are more specific for the subtype of the virus.

### (L)WG(I or V)HHP

(L)WGIHHP and (L)WGVHHP sequences correspond to X31 aminoacids (178) 179-184 and belong to the less available sequences. It does not appear on the surface of virus and would not be useful for regular vaccination use. These peptide sequences and corresponding nucleic acid sequences are, however, useful for analysis of influenza viruses. The sequences are present in practically all influenza A viruses and can be thus used for typing of viruses, especially defining presence of influenza A virus in a sample.

### The Corresponding Nucleic Acid Sequences

Preferred analytical and/or therapeutic tools include corresponding nucleic acid sequences, especially the influenza virus nucleic acid sequences coding the peptide epitopes useful for example DNA/RNA diagnostics and/or for gene therapy/RNAi-methods. Preferred diagnostic methods include known polymerase chain reaction, PCR, methods known for influenza diagnostics (see U.S. Pat. No. 6,811,971 and WO0229118). The preferred nucleic acid sequences include sequences coding amino acid (L)WGIHHP and (L)WGVHHP corresponding to X31 aminoacids (178) 179-184 or part thereof.

### Analysis of Consensus Sequences by a Group of H1-H5 Viruses Form Animals and Human

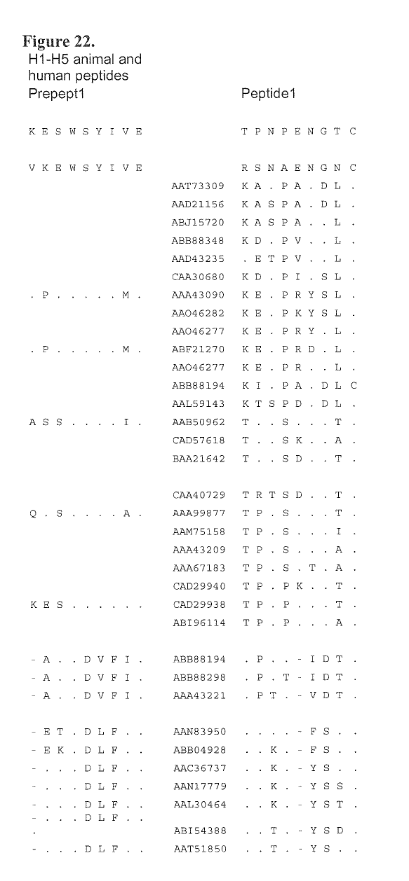

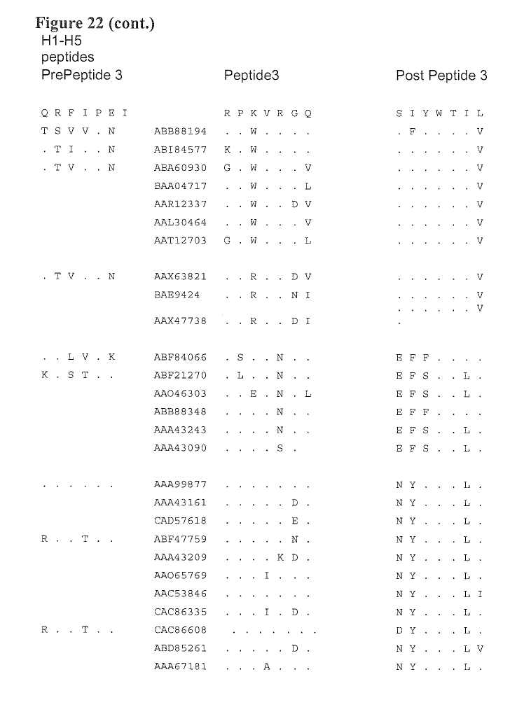

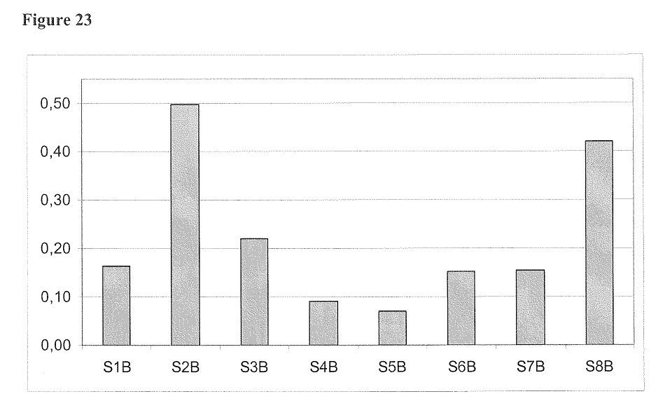

A group of influenza A viruses comprising chicken, duck, swine and human H1-H5 viruses was collected from database. The sequences were aligned and homologies were compared FIG. 22 includes PrePeptide 1, peptide 1, prepeptide 3, peptide 3 and postPeptide 3 from the comparison

Prepeptide 1 from H1-H5 Human and Animal Comparison.

The tables reveal following variant of the peptide, when samples were taken from the search. The sequences revealed to belong to two major groups A and B

The general consensus and preferred prePept1 for group A sequences:

X1 W S Y I X2 E,

wherein X1 is preferably E or S and X2 is A, I, V or M

The preferred sequences are included in following subgroups:

V K E W S Y I V E,

Comprising one characteristic residue E and V from the two following subgroups

V P E W S Y I M E,

associated with specific group of peptides 1, with characteristic proline and methionine

A S S W S Y I I E,

Q K S W S Y I A E

K E S W S Y I A E,

K E S W S Y I V E

(consensus used in H1 analysis)
these forms another group which further includes similar sequences from influenza H1 analysis, The serine in position 3 is characteristic, with one exeption G, which is present e.g. in human Asian strain BAC82843, following four residues WSYI are quite conserved, and second last residue is hydrophobic residue preferably A, I or V and the last residue E is quite conserved. The two first residues are more varying and usually polar or charged. Similar sequences were found from animal viruses.

The general consensus and preferred prePept1 for group B sequences:

A1 E2 W D V3 F I E,

### Wherein

### A1 is A, E or K; E2 is E, T or K; and V3 is V or L

Further including two subgroups

B1

A E W D V F I E,

which is preferably coexpressed with a characteristic
Peptide 1 region and similar type of viruses
And B2 peptides further divided to two groups

B2a

E T1 W D L F I E

Wherein T1 is either T or K and this is preferably present in a group hemagglutinins with specific peptide 1 comprising AFS-epitope

B2b

K E W D L F . . .

Wherein B2b is preferably present in a group hemagglutinins with specific peptide 1 comprising AYS-epitope.

It is realized that the specific pre- or postpeptide or Peptide 1-4 subgroups are useful for the characterization and classification of hemagglutinins. The most conserved sequences and combinations thereof are useful for development of PCR-primers.

### Peptide 1

The peptide 1 sequences were revealed to be present as four major groups A, B, C and D

The consensus sequence for peptide Peptide 1 group A is

K A₁ N₂ P A₃ N₄ D₅ L C

wherein A₁ is A, D, E, I, or T; N₂ is N, S, or T; A₃ is A or V, I, D, R or K; N₄ is N, Y or D; D₅ is D, G or S. Additionally in a variant C may replace sub-carboxyterminal L and amino-terminal K may be replaced by the similar positive charged R. The group A can be further divided to two groups A1 and A1
subgroup A1, wherein A₃ is positively charged residue, preferably R or K, and A₁ is negatively charged residue, preferably E
subgroup A2, wherein A₃ is hydrophobic alkyl-side chain residue, preferably A, V or I, and A₁ is negatively charged residue, preferably E, or D, or hydrophobic residue A; and/or N₂ is optionally S or T

The consensus sequence for peptide Peptide 1 group B is

T S₁ N₂ S₃ E₄ N₅ G T₅ C

wherein S₁ is S, R, or P; N₂ is N, or T; S₃ is S or P; E₄ is charged residue E, K or D; N₅ is N or T; T₅ is T, A or I.

Preferred subgroups of B includes B1 with S₃ is S and B2 wherein S₃ is P having clear conformational differences due to structure of P. In a preferred embodiment N₅ is N, which is common residue in peptides B.

The consensus sequence for peptide Peptide 1 group C is

R P N₁ A₂- I₃ D T C

wherein N₁ is N, or T; A₂ is A, or T; I₃ is hydrophobic aliphatic residue, preferably branched residue, more preferably V or I. This group form a specific group of hemagglutinins with preferred PrePeptide 1 comprising D V F I or very homologous residues.

The consensus sequence for peptide Peptide 1 group D is

R S N₁ A - F₂ S N₃ C

wherein N₁ is N, K, or T; F₂ is an aromatic side chain amino acid, preferably F, or Y; N₃ is polar residue, preferably N, D, S or T. This group form a specific group of hemagglutinins with preferred PrePeptide 1 comprising D L F or very homologous residues.

It is realized that additional few aminoacid residues may be included to amino or carboxy-terminal to improve conformation of the peptide. The elongated peptides may be more useful for database searches. The preferred carboxyterminal additional amino acid residue includes 1-6, more preferably 2-4 and most preferably 3 or 4 amino acid residue consecutive to the peptide 1.

### Total Consensus of Peptide 1

The total consensus sequence for peptide Peptide 1 is

R₁ S₂ N₃ A₄ E₅ N₆ G₇ N₈ C

wherein
R₁ is a polar positively charged or non-charged residue preferably from group R, K, or T;
S₂ is polar residue S, or T; N or D or R: or conformational residue P
N₃ is polar residue S, or T; N or K.
A₄ is polar residue S, or T; or aliphatic small chain A or conformational residue P.
E₅ is polar residue with negative charge E or D, positive charge R or K; or hydrophobic A, V or I or deleted.
N₆ polar residue N, or D; aromatic F or Y; or hydrophobic residue I or V
G₇ is polar residue G, D or S,
N₈ is polar residue S or T, N, or D; or hydrophobic residue A or L.

It is notable that S or T in position 2, 3, 4 and 8 are very similar with polar hydroxyl side chain, T (and thus putatively also S) can be present in position 1 and S in 7; polar positively charged R and K with similar sizes were both observed position 1, 5 and one of these in 2 and 3; negatively charged similar D and E both in position 5 and D in 2, 6, 7, 8 and amide of D derivative N (positions 2, 3, 6 and 8); at least hydrophobic aliphatic A, V, L, I are present in 4, 5, 6, and 8; and aromatic similar Y and F in position 6. Referring positive +, negative −, polar O, P-proline, C-hydrophobic alkyl, B-aromatic) (+ O), (+ − O P), (+ O), (O C P), (+ − C or del), (− O B C), (− O), (− O C), revealing relatively limited actual variation.

Peptide 3 from Animal and Human H1-H5 Peptide Search

### Total Consensus Peptide

R₁ P₂ K₃ V₄ R₅ G₆ Q₇

wherein
R₁ is a polar positively charged group R, K, or non polar small G; or rarely S or I
P₂ is polar residue S, or conformational residue P or hydrophobic L
K₃ is polar charged residue R or K, E or aromatic non-polar residue W.
V₄ is aliphatic hydrophobic aminoacid residue A, V, or I.
R₅ is positively charges R or K; or polar N or S.
G₆ similar polar/negative residue N, or D or E; or small polar G,
Q₇ is polar residue Q, or aliphatic hydrophobic aminoacid residue V, L or I.

Referring positive +, negative −, polar O, P-proline, C-hydrophobic alkyl, B-aromatic) (+ O C), (O C), (+ − B), C, (+ O), (− O), (O C), revealing relatively limited actual variation.

The peptide 3 sequences were revealed to be present as three major groups A, B, and C.

The consensus sequence for peptide Peptide 3 group A is

R₁ P K₂ V R G₆ Q₇

wherein

R₁ is a polar positively charged group R, K, or non polar small G;

K₂ is polar charged residue R or K, E or aromatic non-polar residue W.

G₆ similar polar/negative residue N, or D.
Q7 is polar residue Q, or aliphatic hydrophobic aminoacid residue V, L or I.

The group B is homogenous group of hemagglutinins with characteristic PrePeptide 3 and especially PostPeptide 3 structures.

### The Consensus Sequence for Peptide Peptide 3 Group B is

R P₁ K₂ V N₃ G Q₄

### Wherein

P₁ is polar residue S, or conformational residue P or hydrophobic L
K₂ is polar charged residue K or E
N₃ is positively charges R or K; or polar N or S.
Q₄ is polar residue Q, or aliphatic hydrophobic aminoacid residue L.

The group B is homogenous group of hemagglutinins with characteristic PrePeptide 3 and especially PostPeptide 3 structures.

### The Consensus Sequence for Peptide Peptide 3 Group C is

R P K V₁ R₂ G₃ Q₄

wherein
V₁ is aliphatic hydrophobic aminoacid residue A, V, or I.
R₂ is positively charges R or K.
G₃ similar polar/negative residue N, or D or E; or small polar G,
Q₄ is polar residue Q, or aliphatic hydrophobic aminoacid residue L.

The group B is homogenous group of hemagglutinins with characteristic PrePeptide 3 and especially PostPeptide 3 structures.

Analysis of H3 HA-Consensus Sequences from a Group of Influenza Viruses

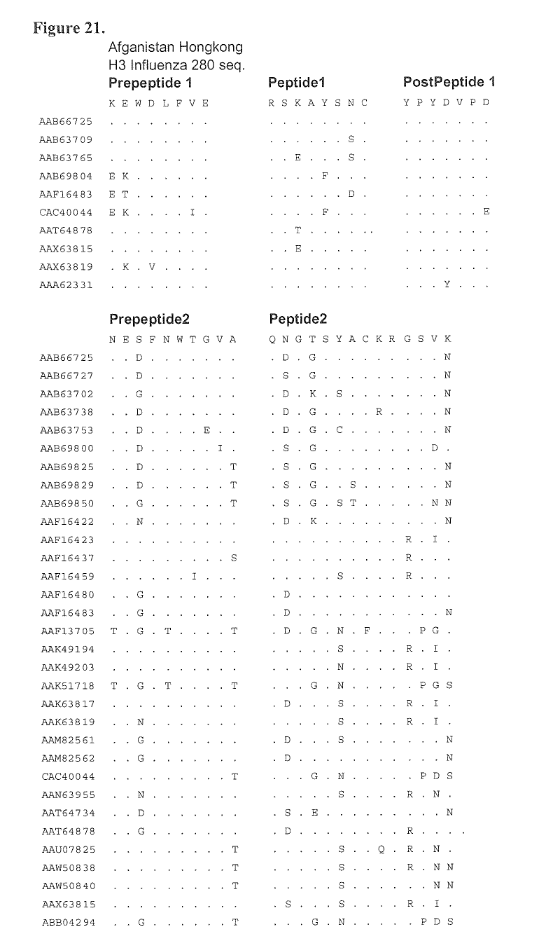

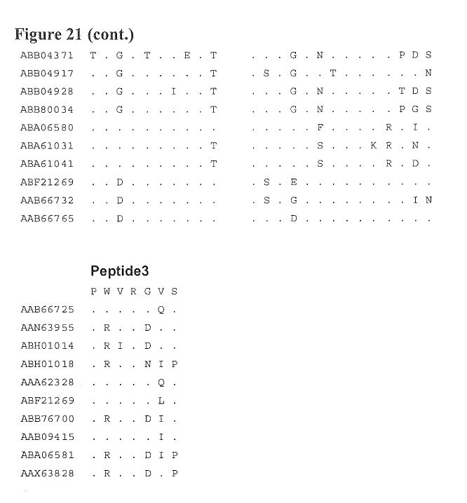

280 H3 sequences was collected and aligned from databank, FIG. 21. The sample sequences were from Honk Kong and Afghanistan, selected as remote places and remote from Finland which was analyzed separately and part of the sequences were added to the consensus. The aligned sequences were compared in order to reveal consensus sequences and collect individual sequence variants.

The invention is especially directed to collecting and grouping of sequence variants in order to classify viruses and reveal groups of viruses with specific antigenic and other functional such as sialylated natural glycan binding properties as studied in the previous applications of the inventors.

### Total Consensus of Peptide 1

The peptides appeared to be homologous, with minor changes

The total consensus sequence for peptide Peptide 1 is

R S K₁ A Y₂ S N₃ C

wherein
K₁ is a polar charged or non-charged residue preferably from group E, K, or T;
Y₂ is aromatic residue Y or F or D (from analysis of Finnish sequences).
N₃ is polar residue S; N or D.

Preferred subgroups of Peptide 1 includes 4 groups A, B C and D

### The Group A Consist of Sequences

R S K A Y S N₃ C

Wherein the polar residue N3 varies as above
This is a characteristic sequence in many recent viruses

### The Group B Consist of Sequences

R S K A F S N C

Which is a characteristic sequence in many viruses.

### The Group C Consist of Sequences

R S K₁ A Y S N₃ C

Wherein the polar residue N3 varies as above and

### K₁ is E or T

### The Group D Consist of Unusual Sequences

R S K₁ A D S N₃ C

Wherein the polar residue N₃ varies as above and
K₁ is as above, or these are more preferably N and K, respectively
Peptide 2 of H3 viruses

Analysis of Finnish sequences gave consensus core peptide sequences SNACKR, SYAKR and SSACKR These the core peptides were compared to ones obtained from the analysis of HongKong/Afganistan viruses The core epitope was elongated by four aminoacid residues to include conserved and binding functional residues and by one residue from carboxy-terminus, further residues in the close region are in the Table.

QN₁GT₂SY₃A₄CK₅R₆G₇

wherein
N₁ is a polar negatively charged or non-charged residue preferably from group D, N and S,
T₂ is polar neutral or charged residue T, G; D, E or K.
Y₃ is polar residue S, N, or C; or aromatic Y or F
A₄ is aliphatic small chain A or similar polar residue S, or T
K₅ is polar residue with positive charge K or R;
R₆ polar residue with positive charge R or K; preferably R
G₇ is polar residue G, or positively charged, preferably R.

Preferred variant groups includes peptides with different Y₃, in four groups

Group A according to formula above, wherein Y₃ is N. This is present in old and some new viruses.

Group B according to formula above, wherein Y₃ is Y or F. This is characteristic with residue Y in part of new/90′ s influenza viruses as in analysis of Finnish viruses.

Group C according to formula above, wherein Y₃ is S. This is characteristic in especially for a group of new influenza viruses observed especially after year 2000 as shown in analysis of Finnish viruses

### Peptide 3 of H3 Influenza Viruses

Analysis of Finnish influenza viruses revealed RPWVRGL, RPWVRGV, RPWVRGI, RPWVRGQ, RPRVRD(V/I/X). The Afganistan/Hongkong viruses were analyzed including one additional residue at carbody terminus of the core sequence, as preferred additional residue.

Consensus sequence of H3 influenza peptides

### R₁PW₂V₃RG₄V₅S₆

wherein
R₁ is a polar positively charged group preferably R, or other G, S or I;
W₂ is large aromatic hydrophobic W or positively charged group. preferably R
V₃ is alkyl hydrophobic residue, preferably V or I.
G₄ is polar residue G, N or D
V₅ is non-charged Q or hydrophobic V, L or I.
S₆ is polar S or conformational P.

Preferred structure groups include common according to the consensus Formula:

Group A wherein R₁ is R and
More rare
group B wherein R₁ is not R and is preferably G, S or I.

The preferred structure

Group C includes Structures according to the consensus Formula above wherein W₂ is W. and
Group D includes peptides according to the consensus Formula, wherein W₂ is not W, preferably being positively charged residue, more preferably R, and also preferably
G₄ is not G, and preferably G₄ is D or N.

### Antigenic Compound

An “antigenic compound” as used herein means a compound, for example a peptide, or a composition of multiple, two or three or more peptides, or peptide like compounds, which can elicit an antigenic reaction in an animal. It is not necessary for an antigenic compound to elicit or raise an immunogenic reaction; it may do so or not. An antigenic compound may be used for the purposes of raising immunogenic response or for screening assays. An antigenic compound comprises an epitope or epitopes which may be or are suitable for eliciting an immunogenic response. Favorably, an antigenic compound, for example, a peptide or peptides conjugated to together, via a peptide sequence or by other means, e.g. covalently, binds an antibody substance and can elicit an immunogenic response in a mammalian subject, e.g. in humans. An antigenic compound can be used in in vitro assays, for example in binding assays when screening antibody substances which bind an antigenic compound or compounds.

Preferably an antigenic compound comprises a peptide selected from the group consisting of K₁V₂R₃, W₁V₂R₃, K₁V₂N₃, T₁P₂N₃P₄E₅N₆G₇T₈, S₁K₂A₃Y₄S₅N₆, K₁A₂N₃P₄A₅N₆D₇L₈, V₁T₂K₃G₄V₅S₆A₇S₈, G₁T₂S₃S₄A₅, E₁A₂S₃S₄G₅V₆S₇S₈A₉, and said peptide corresponding to influenza virus A hemagglutinin. “Corresponds” as used herein means that the amino acids of an antigenic compound are similar or homologous to influenza virus A hemagglutinin amino acids. Skilled artisan understands when peptide of the present invention corresponds influenza virus A hemagglutinin and when the peptide or the antigenic compound is something else than HA or influenza virus A.

Preferably an antigenic compound comprises at least one peptide selected from the group of K₁V₂R₃, W₁V₂R₃, K₁V₂N₃, T₁P₂N₃P₄E₅N₆G₇T₈, S₁K₂A₃Y₄S₅N₆, K₁A₂N₃P₄A₅N₆D₇L₈, V₁T₂K₃G₄V₅S₆A₇S₈, G₁T₂S₃S₄A₅, E₁A₂S₃S₄G₅V₆S₇S₈A₉.

In another favorable embodiment an antigenic compound comprises at least two peptides selected from the group consisting of K₁V₂R₃, W₁V₂R₃, K₁V₂N₃, T₁P₂N₃P₄E₅N₆G₇T₈, S₁K₂A₃Y₄S₅N₆, K₁A₂N₃P₄A₅N₆D₇L₈, V₁T₂K₃G₄V₅S₆A₇S₈, G₁T₂S₃S₄A₅, E₁A₂S₃S₄G₅V₆S₇S₈A₉.

In more preferred embodiment an antigenic compound comprises at least three peptides selected from the group consisting of K₁V₂R₃, W₁V₂R₃, K₁V₂N₃, T₁P₂N₃P₄E₅N₆G₇T₈, S₁K₂A₃Y₄S₅N₆, K₁A₂N₃P₄A₅N₆D₇L₈, V₁T₂K₃G₄V₅S₆A₇S₈, G₁T₂S₃S₄A₅, E₁A₂S₃S₄G₅V₆S₇S₈A₉.

In more preferred embodiment the peptide K₁V₂R₃ according to claim 1, wherein K₁ is an optional residue of an amino acid selected from the group of K, E, M and conservative substitutes thereof; V₂ stands for a residue of an amino acid selected from the group of V, I, L, F, A and conservative substitutes thereof; and R₃ is a residue of an amino acid selected from the group of R, K and N and conservative substitutes thereof.

In more preferred embodiment the peptide W₁V₂R₃ according to claim 1, wherein Wᵢ is an optional residue of an amino acid selected from the group of W, R, L, K and conservative substitutes thereof; V₂ stands for a residue of an amino acid selected from the group of V, I, A, E, G and conservative substitutes thereof; and R₃ is a residue of an amino acid selected from the group of R and conservative substitutes thereof.

In more preferred embodiment the peptide K₁V₂N₃ according to claim 1, wherein K₁ is an optional residue of an amino acid selected from the group of K, E, R, Q, M and conservative substitutes thereof; V₂ stands for a residue of an amino acid selected from the group of V, I, L, F, A and conservative substitutes thereof; and N₃ is a residue of an amino acid selected from the group of N, R, K, D and conservative substitutes thereof.

In more preferred embodiment the peptide T₁P₂N₃P₄E₅N₆G₇T₈ according to claim 1, wherein T₁ is an optional residue of an amino acid selected from the group of T, K, A, P and conservative substitutes thereof; P₂ stands for a residue of an amino acid selected from the group of P, S, K, T and conservative substitutes thereof; N₃ is a residue of an amino acid selected from the group of N, D, S, T and conservative substitutes thereof; P₄ is a residue of an amino acid selected from the group of P, S, C, A, T and conservative substitutes thereof; E₅ is a residue of an amino acid selected from the group of E, K, D, G, Y and conservative substitutes thereof; N₆ is a residue of an amino acid selected from the group of N, Y, T and conservative substitutes thereof; G₇ is a residue of an amino acid selected from the group of G and conservative substitutes thereof; and T₈ is a residue of an amino acid selected from the group of T, I, A, V, K and conservative substitutes thereof.

In more preferred embodiment the peptide S₁K₂A₃Y₄S₅N₆ according to claim 1, wherein S₁ is an optional residue of an amino acid selected from the group of S, N, R, G, T, D and conservative substitutes thereof; K₂ stands for a residue of an amino acid selected from the group of K, T, R, N, I, E, S and conservative substitutes thereof; A₃ is a residue of an amino acid selected from the group of A and conservative substitutes thereof; Y₄ is a residue of an amino acid selected from the group of Y, F, H, T, S and conservative substitutes thereof; S₅ is a residue of an amino acid selected from the group of S, Q and conservative substitutes thereof; N₆ is a residue of an amino acid selected from the group of N, D, T, S, I, V and conservative substitutes thereof.

In more preferred embodiment the peptide K₁A₂N₃P₄A₅N₆D₇L₈ according to claim 1, wherein K₁ is an optional residue of an amino acid selected from the group of K, R and conservative substitutes thereof; A₂ stands for a residue of an amino acid selected from the group of A, T, P, I, V, D, N and conservative substitutes thereof; N₃ is a residue of an amino acid selected from the group of N, S, D, K, I and conservative substitutes thereof; P₄ is a residue of an amino acid selected from the group of P, T and conservative substitutes thereof; A₅ is a residue of an amino acid selected from the group of A, V, T, P, I, S and conservative substitutes thereof; N₆ is a residue of an amino acid selected from the group of N, K, Y, D and conservative substitutes thereof; D₇ is a residue of an amino acid selected from the group of D, G, F and conservative substitutes thereof; and L₈ is a residue of an amino acid selected from the group of L, P, R, M and conservative substitutes thereof.

In more preferred embodiment the peptide V₁T₂K₃G₄V₅S₆A₇S₈ according to claim 1, wherein V₁ is an optional residue of an amino acid selected from the group of V, I, T, Q, A and conservative substitutes thereof; T₂ stands for a residue of an amino acid selected from the group of T, S, L, N, I, K, F and conservative substitutes thereof; K₃ is a residue of an amino acid selected from the group of K, R, G, I and conservative substitutes thereof; G₄ stands for a residue of an amino acid selected from the group of G and conservative substitutes thereof; V₅ stands for a residue of an amino acid selected from the group of V, G, A, I, T and conservative substitutes thereof; S₆ stands for a residue of an amino acid selected from the group of S, T, M and conservative substitutes thereof; A₇ stands for a residue of an amino acid selected from the group of A, T, V, K, S, D and conservative substitutes thereof; and S₈ stands for a residue of an amino acid selected from the group of S, A and conservative substitutes thereof.

In more preferred embodiment the peptide G₁T₂S₃S₄A₅ according to claim 1, wherein G₁ is an optional residue of an amino acid selected from the group of G, E, R and conservative substitutes thereof; T₂ stands for a residue of an amino acid selected from the group of T, G, E, D, K, I, S, A and conservative substitutes thereof; S₃ is a residue of an amino acid selected from the group of S, G, T and conservative substitutes thereof;

S₄ stands for a residue of an amino acid selected from the group of S, Y, C, N, F, D, G, P, A, H and conservative substitutes thereof; and A₅ is a residue of an amino acid selected from the group of A, S, T, G and conservative substitutes thereof.

In more preferred embodiment the peptide E₁A₂S₃S₄G₅V₆S₇S₈A₉ according to claim 1, wherein E₁ is an optional residue of an amino acid selected from the group of E, D, V, G, N, Y and conservative substitutes thereof; A₂ stands for a residue of an amino acid selected from the group of A, V, S, T, P and conservative substitutes thereof; S₃ is a residue of an amino acid selected from the group of S, T and conservative substitutes thereof; S₄ stands for a residue of an amino acid selected from the group of S, L, V and conservative substitutes thereof; G₅ stands for a residue of an amino acid selected from the group of G, W and conservative substitutes thereof; V₆ stands for a residue of an amino acid selected from the group of V, L, G and conservative substitutes thereof; S₇ stands for a residue of an amino acid selected from the group of S, R and conservative substitutes thereof; and S₈ stands for a residue of an amino acid selected from the group of S, A and conservative substitutes thereof; and A₉ stands for a residue of an amino acid selected from the group of A, V and conservative substitutes thereof.

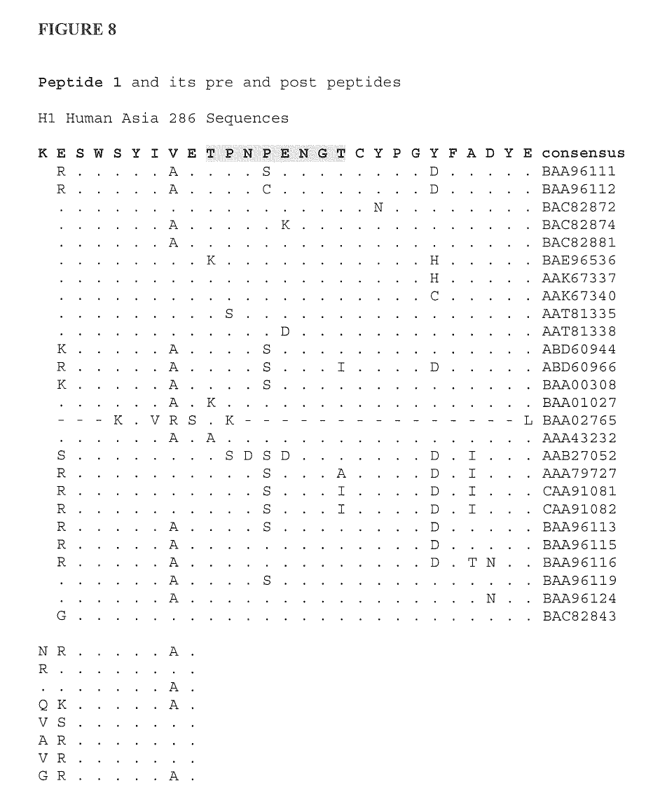

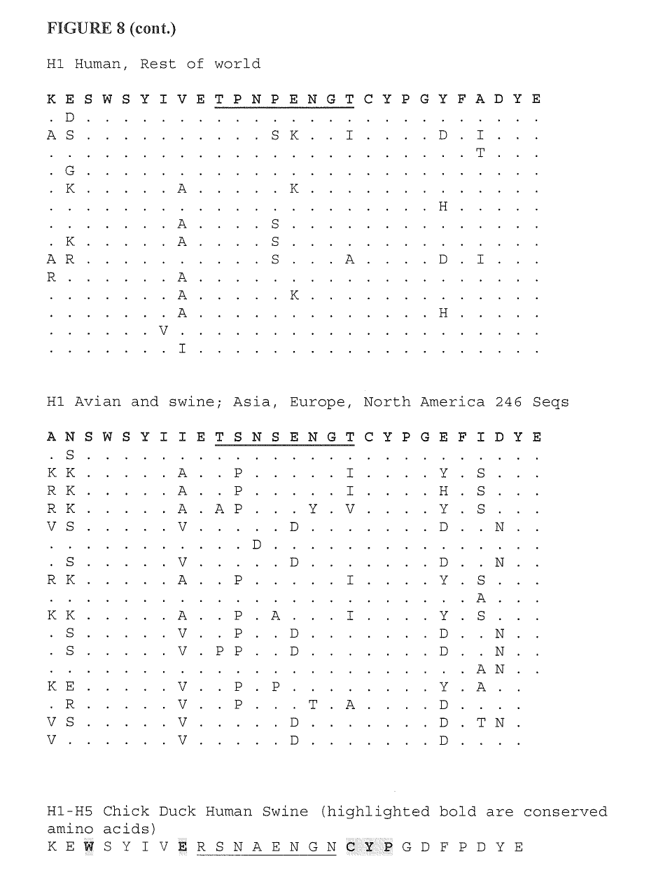

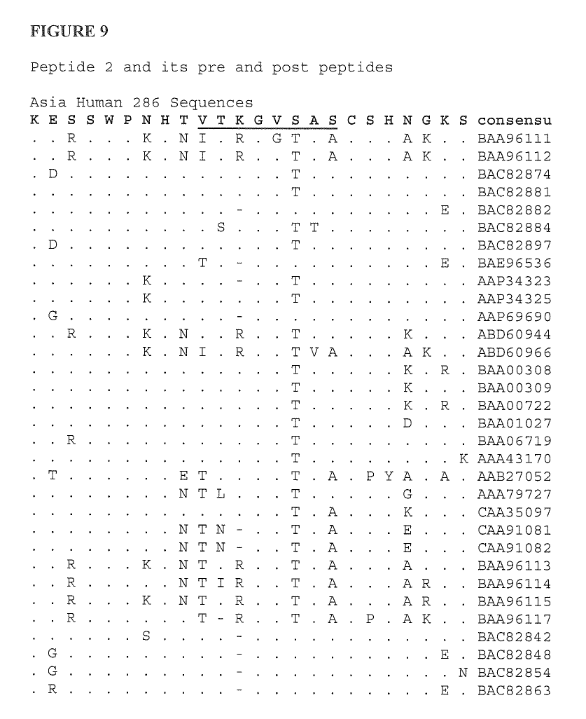

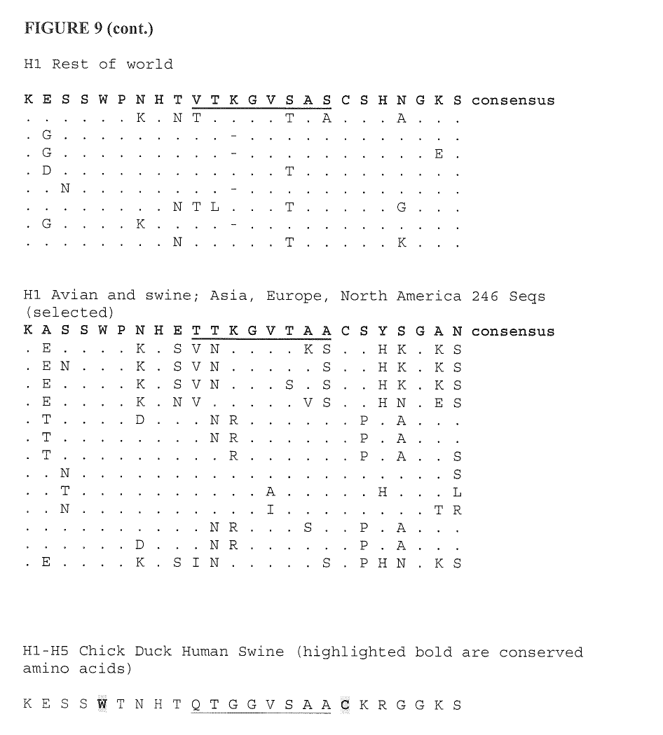

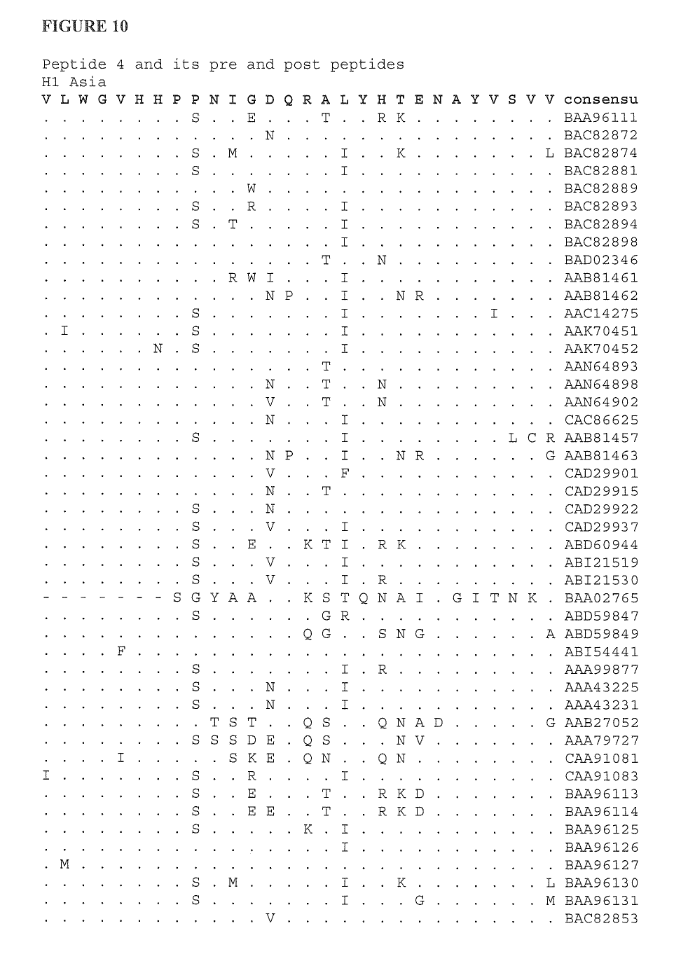

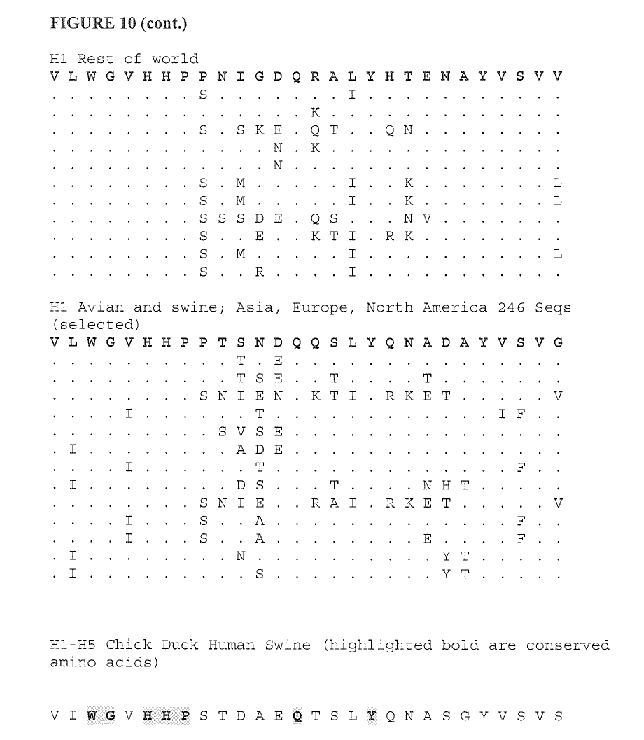

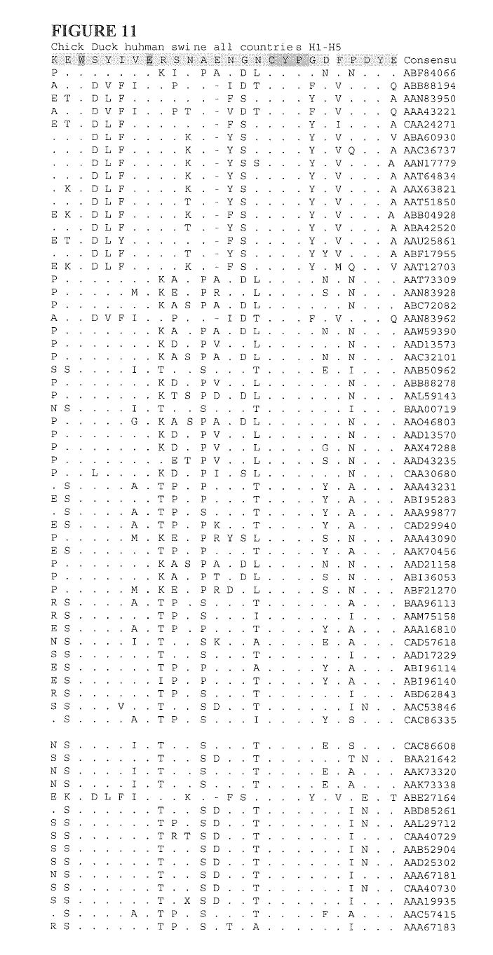

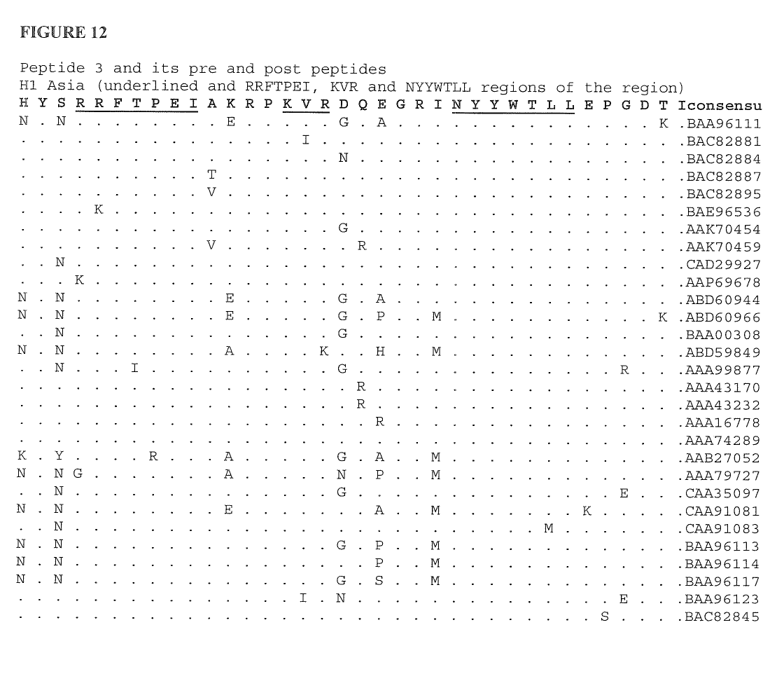

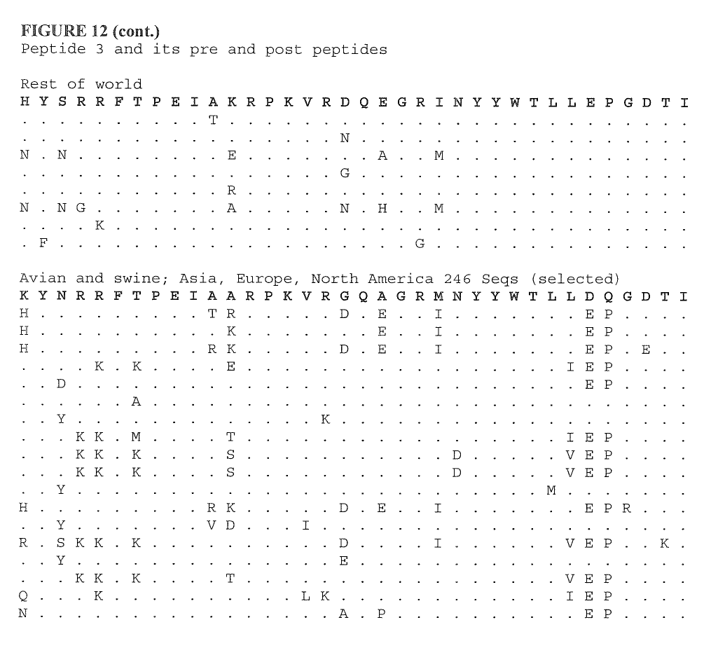

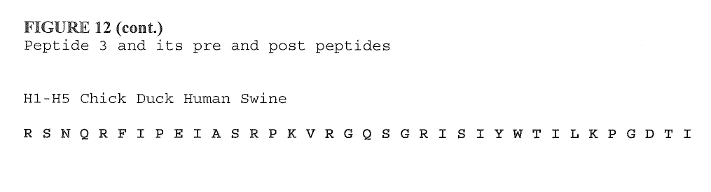

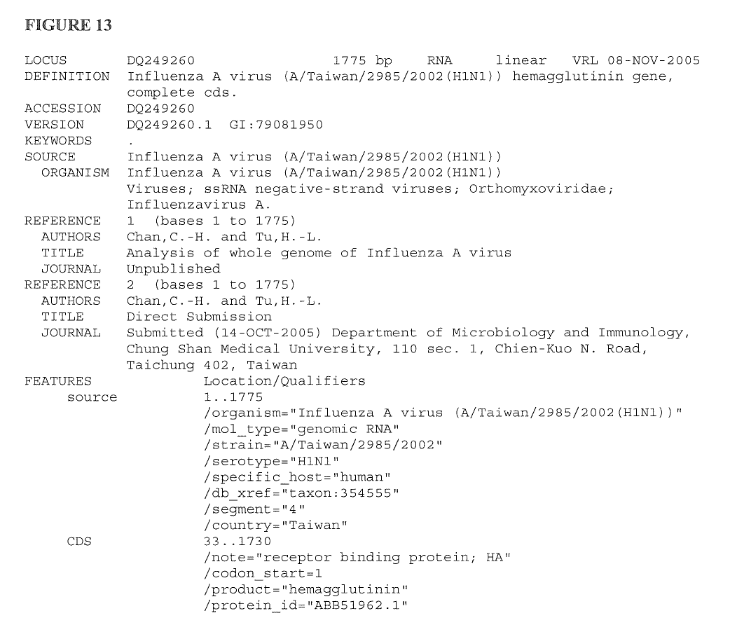

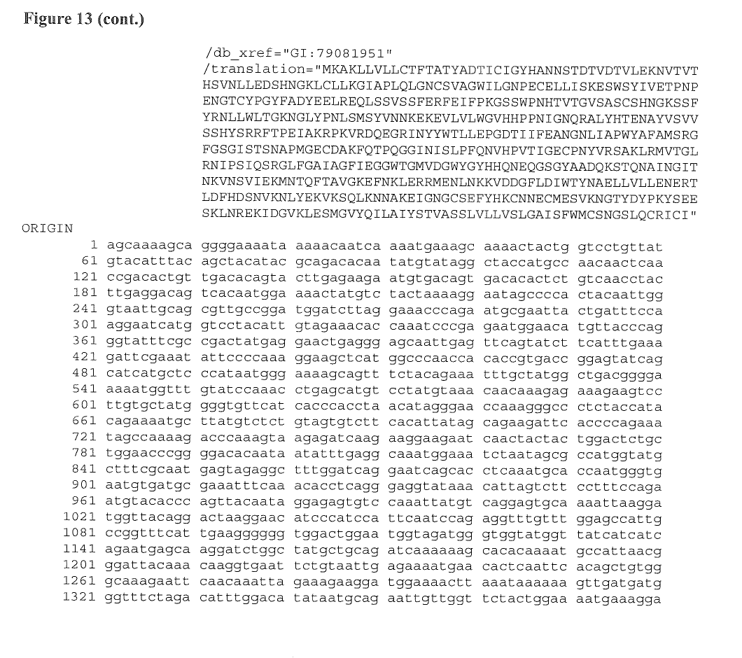

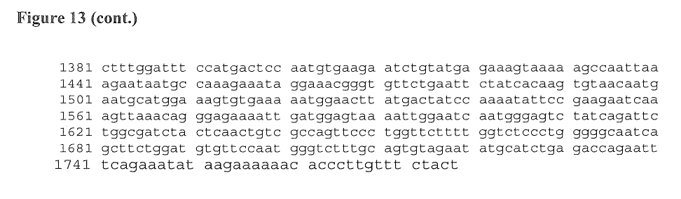

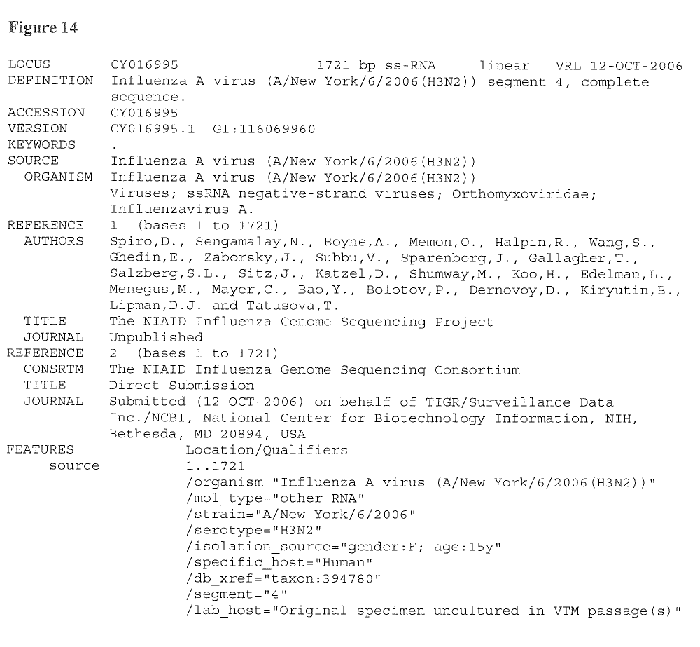

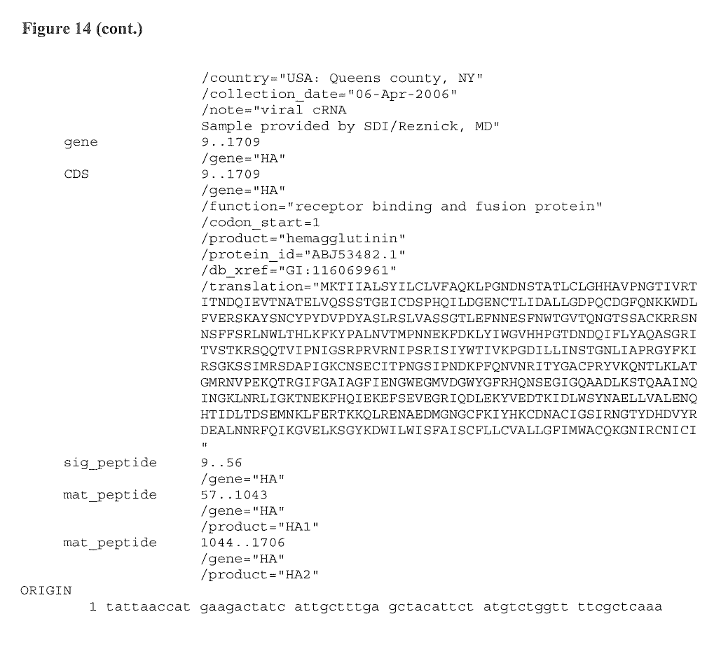

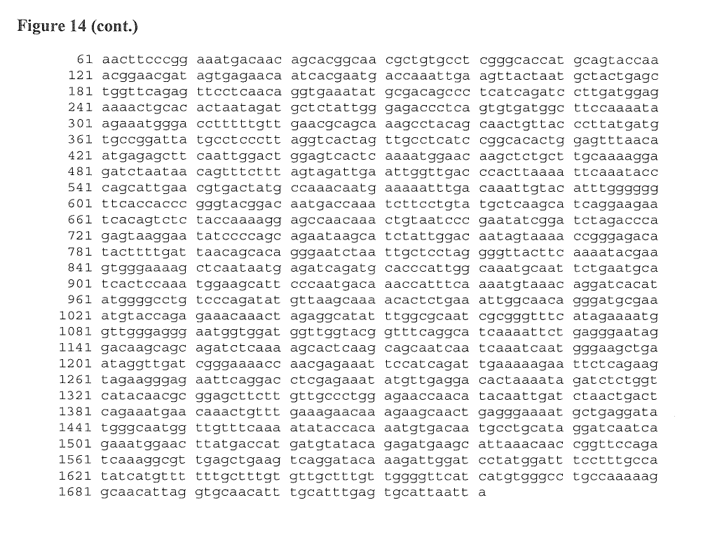

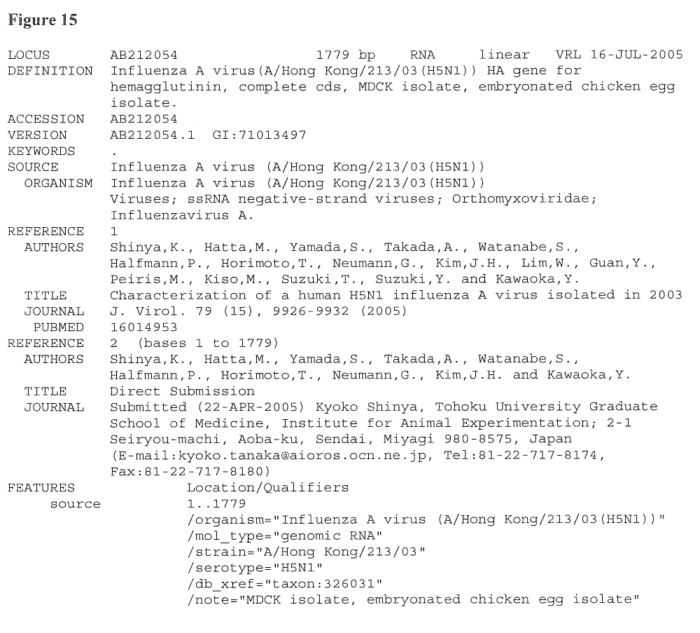

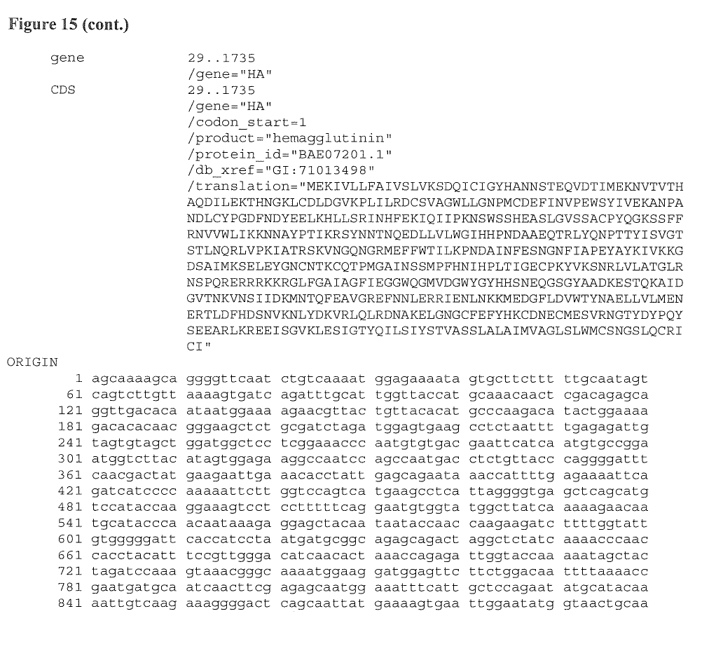

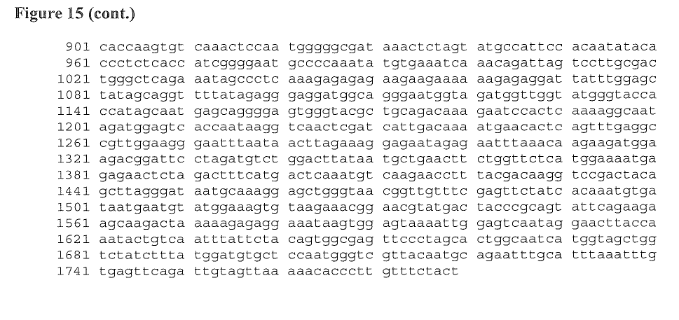

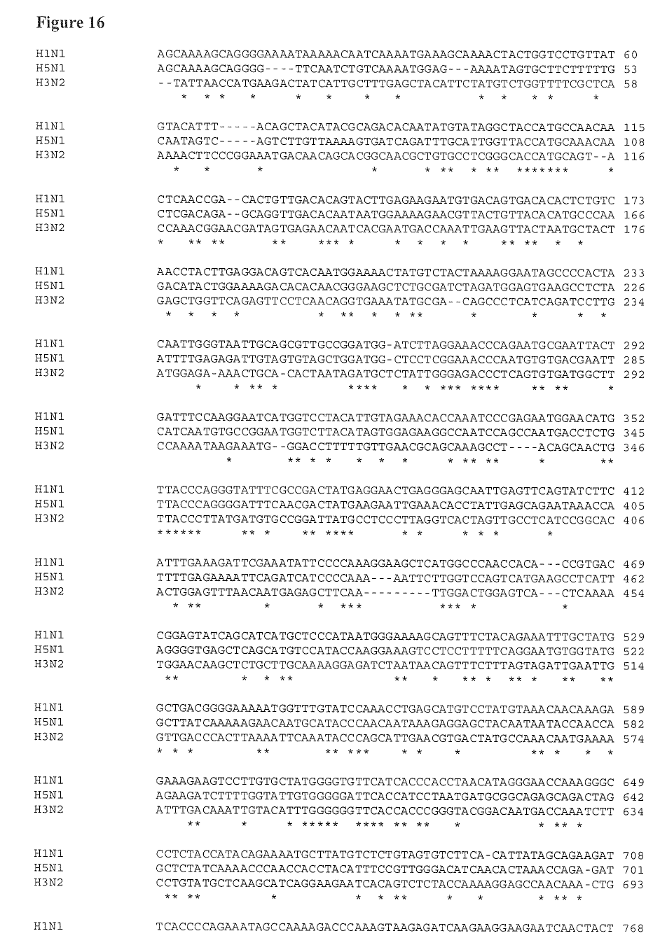

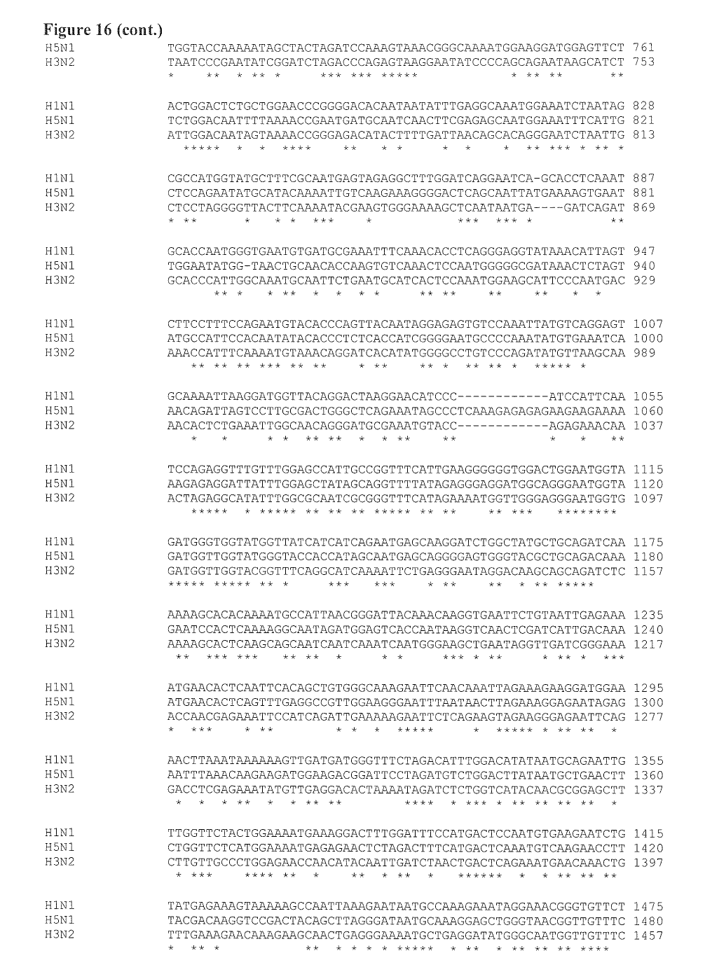

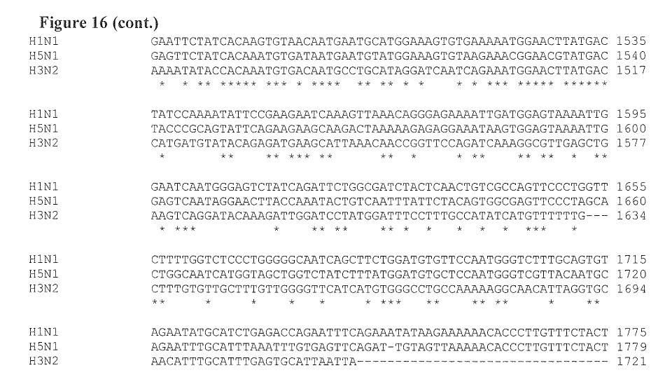

Even in more preferred embodiment a peptide is selected from the group consisting of KVR, WVR, KVN, TPNPENGT, TSNSENGT, RSNAENGN, SKAYSN, SNAFSN, KANPANDL, VTKGVSAS, TTKGVTAA, QTGGVSAA, EASSGVSSA, GTSSA, GGSNA, GTSYA and any natural HA peptide sequence comprising 3-9 amino acids in FIGS. 8-12. Any peptide sequence can be selected from the naturally occurring HA sequences. It is also anticipated that new variants emerge from the natural sequences and the present invention is, in more preferred embodiment, suited for new variants, e.g. H5N1, which infect humans. H5N1 antigenic compounds are preferred embodiments of the present invention.

The present invention embraces also pre and post peptide regions that flank peptide 1, 2, 3, and 4 regions. In some applications these regions are well suited for use of primers directed to amplify or detect antigenic compounds of the present invention. In some applications certain antibody substances can be used concomitantly with antigenic compounds of the present invention. An “antigenic compound” as used herein encompass pre and post peptide amino acid and nucleic acid sequences, typically 2-9 aa or 6-27 by of length.

An antigenic compound comprises preferably 5 to 13 amino acids. The antigenic compound can be shorter, e.g. 3 or 4 amino acids, or it can be longer, 6, 7, 8, 9, 10, 11, or 12 amino acids. The prior art teaches long antigenic peptides derived from influenza virus A but in the present invention inventors have discovered that short amino acid sequences are better to e.g. screen natural antibodies and elicit an immunogenic response.

The preferred influenza virus A hemagglutinin subtypes according to invention are hemagglutinin (HA) subtypes H1, H3 and H5. Even more preferred subtypes are H1N1, H3N2 and H5N1.

Preferably an antigenic compound comprises at least two peptides as defined in claim 1. In some applications it is beneficial to include two peptides, which together enhance the binding efficiency of an antibody substance and inhibition of influenza virus binding to epithelial cells or target cells influenza virus infects. An antigenic compound comprising at last two peptides is even more preferred antigenic compound.

In more preferred embodiment the antigenic compound comprises at least three peptides as defined in claim 1. In some applications it is beneficial to include three peptides, which together enhance the binding efficiency of an antibody substance and inhibition of influenza virus binding to epithelial cells or target cells influenza virus infects. Antibody substances binding to or recognizing three peptides of the present invention are potent inhibitors of influenza virus. An antigenic compound comprising at last three peptides is preferred antigenic compound of the present invention.

The present invention embraces also a method for producing a vaccine against influenza virus. Preferred steps comprise preparing an antigenic compound comprising at least one peptide according to claim 1; administering said compound to an animal; and monitoring the animal in order to detect immune response against the antigenic compound.

In more preferred embodiment an antigenic compound comprises at least two peptides according to claim 1.

Preferably, an antigenic compound used for a vaccine comprises a carrier, other immunogenic peptides, or an adjuvant. Even more preferably, the peptide is covalently linked to the surface of a carrier protein.

The invention contemplates a vaccine composition comprising an antigenic compound.

Vaccination is preferably performed before anticipated influenza virus infection in a mammalian or human subject. Vaccination can also be done for other animal hosts of influenza virus, e.g. avian or swine species. By this mean eradication or prevention of influenza virus spread in animal populations is prevented or diminished.

Invention also contemplates a method for screening a binding agent against influenza virus HA. Screening method comprises steps of selecting an antigenic compound according to claim 1, assaying binding between antigenic compound and the binding agent; and monitoring the binding of the antigenic compound and binding agent.

The present invention contemplates a method of identifying influenza virus in a biological sample, the method comprising: (a) contacting the biological sample with an antibody substance capable of binding antigenic compound according to claim 1; and (b) detecting the binding between said antibody substance and antigenic compound in the sample, said binding indicating the presence and type of influenza virus in the sample.

The above method is preferred method for detecting influenza virus A HA in a sample.

Binding agent can be an antibody substance as described herein. Binding agent can be a sugar molecule and the binding assay can comprise a modulatory agent, e.g. sugar or oligosaccharide that binds to HA or target cells of HA binding, and effect of modulatory agent is monitored on binding between antigenic compound and binding agent. Skilled artisan know several in vitro and in vivo methods to assay screening of binding agents and binding between binding agent and antigenic compound of the present invention. Exemplary assays are represented in U.S. Pat. No. 7,067,284, U.S. Pat. No. 7,063,943 by Cambridge Antibody Tech, WO2006055371, US2006205089 by Univ. Montana, which are incorporated here in their entirety.

Preferably, binding agents for a library, e.g. antibody library or phage display library, and an antigenic compound is exposed to constituents of the library in conditions favorable for interaction between binding agent and antigenic compound. Libraries of the present invention comprise phage display libraries in which antigenic compounds of the present invention are incorporated or antibody libraries, e.g. U.S. Pat. No. 7,067,284, U.S. Pat. No. 7,063,943 by Cambridge Antibody Tech, WO2006055371. In more preferred embodiment antibody substance is a human antibody, preferably IgM and/or IgG. In more preferred embodiment screening is performed in human serum. By this mean natural antibodies from a human subject or subjects can be assayed and/or screened. Further, these antibodies can be sequenced, produced and administered to human patients infected by an influenza virus or before anticipated infection.

The present invention also

The term “amino acid” as used herein means an organic compound containing both a basic amino group and an acidic carboxyl group. Included within this term are natural amino acids (e.g., L-amino acids), modified and unnatural amino acids (e.g. β-alanine), as well as amino acids which are known to occur biologically in free or combined form but usually do not occur in proteins. Included within this term are modified and unusual amino acids, such as those disclosed in, for example, Roberts and Vellaccio, 1983, the teaching of which is hereby incorporated by reference. Genetically coded, “natural” amino acids occurring in proteins include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include, but are not limited to arginosuccinic acid, citrulline, cysteine sulfmic acid, 3,4-dihydroxyphenylalanine, homocysteine, homoserine, ornithine, 3-monoiodo tyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, norleucine, N-methylaminobutyric acid, naphthylalanine, phenylglycine, 9-phenylproline, tert-leucine, 4-aminocyclohexylalanine, N-methyl-norleucine, 3,4-dehydroproline, N,N-dimethyl-aminoglycine, N-methylaminoglycine, 4-aminopiperidine-4-carboxylic acid, 6-amino-caproic acid, trans-4-(aminomethyl)-cyclohexanecarboxylic acid, 2-, 3-, and 4-(amino˜methyl)-benzoic acid, 1-aminocyclopentanecarboxylic acid, 1-amino cyclopropane-carboxylic acid, and 2-benzyl-5-aminopentanoic acid.

Generally, “peptide” stands for a strand of several amino acids bonded together by amide bonds to form a peptide backbone. The term “peptide”, as used herein, includes compounds containing both peptide and non-peptide components, such as pseudopeptide or peptidomimetic residues or other non-amino acid components. Such a compound containing both peptide and non-peptide components may also be referred to as a “peptide analog”.

The terms “conservative substitution” and “conservative substitutes” as used herein denote the replacement of an amino acid residue by another, biologically similar residue with respect to hydrophobicity, hydrophilicity, cationic charge, anionic charge, shape, polarity and the like. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of argmine for lysine, glutamic acid for aspartic acid, or glutamine for asparagine, and the like. Neutral hydrophilic amino acids, which can be substituted for one another, include asparagine, glutamine, serine and threonine. The term “conservative substitution” also includes the use of a substituted or modified amino acid in place of an unsubstituted parent amino acid provided that substituted peptide reacts with hK2. By “substituted” or “modified” the present invention includes those amino acids that have been altered or modified from naturally occurring amino acids.

Administration of the compositions can be systemic or local and may comprise a single site injection of a therapeutically effective amount of the peptide composition of the present invention. Any route known to those of skill in the art for the administration of a therapeutic composition of the invention is contemplated including for example, intravenous, intramuscular, subcutaneous or a catheter for long-term administration. Alternatively, it is contemplated that the therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

The peptides of the invention will be used as therapeutic or vaccine compositions either alone or in combination with other therapeutic agents. For such therapeutic uses small molecules are generally preferred because the reduced size renders such peptides more accessible for uptake by the target. It is contemplated that the preferred peptides of the present invention are from about 6, 7, 8, 9, or 10 amino acid residues in length to about 90 or 100 amino acid residues in length. Of course it is contemplated that longer or indeed shorter peptides also may prove useful. Thus, peptides of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 and a 100 amino acids in length will be particularly useful. Such peptides may be present as individual peptides or may coalesce into dimers or multimers for greater efficacy.

The polypeptides of the invention include polypeptide sequences that have at least about 99%, at least about 95%, at least about 90%, at least about 85%, at least about 80%, at least about 75%, at least about 70%, at least about 65%, at least about 60%, at least about 55%, at least about 50%, or at least about 45% identity and/or homology to the preferred polypeptides of the invention, the GDNF precursor-derived neuropeptides or homologs thereof.

An “antibody substance” as used herein refers to any antibody or molecule comprising all or part of an antigen-binding site of an antibody and that retains immunospecific binding of the original antibody. Antibody-like molecules such as lipocalins that do not have CDRs but that behave like antibodies with specific binding affinity for the peptides of the present invention also can be used to practice this invention and are considered part of the invention. Antibody substances of the invention include monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention, fragments of the foregoing, and polypeptide molecules that include antigen binding portions and retain antigen binding properties. As described herein, antibody substances can be derivitized with chemical modifications, glycosylation, and the like and retain antigen binding properties.

### Peptide Vaccines and Use Thereof.

### Peptide Vaccine Compositions

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, and Sambrook, et in Molecular Cloning, A Laboratory Manual, Cold Spring Harbor Laboratory Press, 1989.

The application discloses a method of inducing an immune response against a peptide of region B of X31 hemagglutinin. This can be accomplished by conjugating the peptide with a carrier molecule prior to administration to a subject.

In the methods disclosed herein, an immunologically effective amount of one or more immunogenic peptides derivatized to a suitable carrier molecule, e.g., a protein is administered to a patient by successive, spaced administrations of a vaccine composed of peptide or peptides conjugated to a carrier molecule, in a manner effective to result in an improvement in the patient's condition.

In an exemplary embodiment, immunogenic peptides are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the immunogenic peptide. In other cases, there is a linker molecule inserted between the carrier molecule and the immunogenic peptide.

In one exemplary embodiment, the coupling reaction requires a free sulfhydryl group on the peptide. In such cases, an N-terminal cysteine residue is added to the peptide when the peptide is synthesized.

In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Exemplary carrier molecules include proteins such as keyhole limpet hemocyanin (KLH), bovine serum albumin (BSA), flagellin, influenza subunit proteins, tetanus toxoid (TT), diphtheria toxoid (DT), cholera toxoid (CT), a variety of bacterial heat shock proteins, glutathione reductase (GST), or natural proteins such as thyroglobulin, and the like. One of skill in the art can readily select an appropriate carrier molecule.

In a preferred embodiment an immunogenic peptide is conjugated to diphtheria toxin (DT).

In some cases, the carrier molecule is a non-protein, such as Ficoll 70 or Ficoll 400 (a synthetic copolymer of sucrose and epichlorohydrin), a polyglucose such as Dextran T 70.

Another preferred category of carrier proteins is represented by virus capsid proteins that have the capability to self-assemble into virus-like particles (VLPs). Examples of VLPs used as peptide carriers are hepatitis B virus surface antigen and core antigen (Pumpens et al., “Evaluation of and frCP virus-like particles for expression of human papillomavirus 16 E7 oncoprotein epitopes”, Intervirology, Vol. 45, pp. 24-32, 2002), hepatitis E virus particles (Niikura et al., “Chimeric recombinant hepatitis E virus-like particles as an oral vaccine vehicle presenting foreign epitopes”, Virology, Vol. 293, pp. 273-280, 2002), polyoma virus (Gedvilaite et al., “Formation of Immunogenic Virus-like particles by inserting epitopes into surface-exposed regions of hamster polyomavirus major capsid protein”, Virology, Vol. 273, pp. 21-35, 2000), and bovine papilloma virus (Chackerian et al., “Conjugation of self-antigen to papillomavirus-like particles allows for efficient induction of protective autoantibodies”, J. Clin. Invest., Vol. 108 (3), pp. 415-423, 2001). More recently, antigen-presenting artificial VLPs were constructed to mimic the molecular weight and size of real virus particles et al., “Construction of artificial virus-like particles exposing HIV epitopes and the study of their immunogenic properties”, Vaccine, pp. 386-392, 2003).

A peptide vaccine composition may comprise single or multiple copies of the same or different immunogenic peptide, coupled to a selected carrier molecule. In one aspect of this embodiment, the peptide vaccine composition may contain different immunogenic peptides with or without flanking sequences, combined sequentially into a polypeptide and coupled to the same carrier. Alternatively, immunogenic peptides, may be coupled individually as peptides to the same or a different carrier, and the resulting immunogenic peptide-carrier conjugates blended together to form a single composition, or administered individually at the same or different times.

For example, immunogenic peptides may be covalently coupled to the diphtheria toxoid (DT) carrier protein via the cysteinyl side chain by the method of Lee A. C. J., et al., 1980, using approximately 15-20 peptide molecules per molecule of diphtheria toxoid (DT).

In general, derivatized peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the present invention. One preferred vehicle for administration comprises a mixture of mannide monooleate with squalane and/or squalene. Squalene is preferred to squalane for use in the vaccines of the invention, and preferably the ratio of squalene and/or squalane per part by volume of mannide monooleate is from about 4:1 to about 20:1.

To further increase the magnitude of the immune response resulting from administration of the vaccine, an immunological adjuvant is included in the vaccine formulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs. Preferred adjuvants for inclusion in an peptide vaccine composition for administration to a patient are norMDP analogs, such as N-acetyl-nor-muranyl-L-alanyl-D-isoglutamine, N-acetyl-muranyl-(6-O-stearoyl)-L-alanyl-D-isoglutamine, and N-Glycol-muranyl-LalphaAbu-D-isoglutamine (Ciba-Geigy Ltd.). In most cases, the mass ratio of the adjuvant relative to the peptide conjugate is about 1:2 to 1:20. In a preferred embodiment, the mass ratio of the adjuvant relative to the peptide conjugate is about 1:10. It will be appreciated that the adjuvant component of the peptide vaccine may be varied in order to optimize the immune response to the immunogenic epitopes therein.

Just prior to administration, the immunogenic peptide carrier protein conjugate and the adjuvant are dissolved in a suitable solvent and an emulsifying agent or vehicle, is added.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides of the invention. Controlled release preparations may be achieved by the use of polymers to complex or absorb the peptides or antibodies. Controlled delivery may accomplished using macromolecules such as, polyesters, polyamino acids, polyvinyl pyrrolidone, ethylenevinylacetate, methylcellulose, carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the peptide vaccine is entrapped in microcapsules, liposomes, albumin microspheres, microemulsions, nanoparticles, nanocapsules, or macroemulsions, using methods generally known to those of skill in the art.

### Vaccination

The vaccine of the present invention can be administered to patient by different routes such as intravenous, intraperitoneal, subcutaneous, intramuscular, or orally. A preferred route is intramuscular or oral. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats.

It is expected that a dose would consist of the range of to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with influenza virus. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at or 2 months, a third dose at either 4, 6 or 12 months, and additional booster doses at distant times as needed.

A patient or subject, as used herein, is an animal. Mammals and birds, particularly fowl, are suitable subjects for vaccination. Preferably, the patient is a human. A patient can be of any age at which the patient is able to respond to inoculation with the present vaccine by generating an immune response. The immune response so generated can be completely or partially protective against disease and debilitating symptoms caused by infection with influenza virus.

### Evaluation of the Immune Response

In one aspect, the invention provides a means for classifying the immune response to peptide vaccine, e.g., 9 to 15 weeks after administration of the vaccine; by measuring the level of antibodies against the immunogenic peptide of the vaccine.

The invention thus includes a method of monitoring the immune response to the peptide(s) by carrying out the steps of reacting a body-fluid sample with said peptide(s), and detecting antibodies in the sample that are immunoreactive with each peptide. It is preferred that the assay be quantitative and accordingly be used to compare the level of each antibody in order to determine the relative magnitude of the immune response to each peptide.

The methods of the invention are generally applicable to immunoassays, such as enzyme linked immunosorbent assay (ELISAs), radioimmunoassay (RIA), immunoprecipitation, Western blot, dot blotting, FACS analyses and other methods known in the art.

In one preferred embodiment, the immunoassay includes a peptide antigen immobilized on a solid support, e.g., an ELISA assay. It will be appreciated that the immunoassay may be readily adapted to a kit format exemplified by a kit which comprises: (A) one or more peptides of the invention bound to a solid support; (B) a means for collecting a sample from a subject; and (C) a reaction vessel in which the assay is carried out. The kit may also comprise labeling means, indicator reaction enzymes and substrates, and any solutions, buffers or other ingredients necessary for the immunoassay.

### Diagnosis of Influenza Infection

The present invention is also directed to diagnosis of an influenza infection. General methods for diagnosis of an influenza infection are well known to a skilled artisan and are disclosed for instance in U.S. Pat. No. 6,811,971. The present invention provides a method of identifying influenza virus in a biological sample by (a) contacting the biological sample with a nucleic acid primers amplifying the part of virus genome encoding for the divalent sialo side binding site of the X31-hemagglutinin protein as disclosed below under conditions allowing polymerase chain reaction; and (b) determining the sequence of the amplified nucleic acid in the biological sample, to thereby identify the presence and type of influenza virus. Alternatively, the presence of influenza virus can be detected by (a) contacting the biological sample with an antibody or antibody fragment specifically recognizing the divalent sialoside binding site of the X31-hemagglutinin protein as disclosed below; and (b) detecting immunocomplexes including said antibody or antibody fragment in the biological sample, to thereby identify the presence and type of influenza virus in the biological sample.

### The Large Polylactosamine Epitopes: High Affinity Ligands for Influenza Virus

The present invention is directed to a peptide epitopes of hemagglutinin protein of influenza virus derived from the high affinity binding site for sialylated ligands The inventors have previously found out that the influenza virus hemagglutinin bind complex human glycans such as poly-N-acetyllactosamine type carbohydrates using a large binding site according to the invention on its surface, WO2005/037187. The present invention is especially directed to special short peptide epitopes and combinations thereof derived from the large binding site. The special large poly-N-acetyllactosamines are called here “the large polylactosamine epitopes”.

### The Large Binding Site

Furthermore, the present invention is especially directed to the novel large binding site on surface of hemagglutinin, called here “the large binding site”. The large binding site binds effectively special large polylactosmine type structures and analogs and derivatives thereof with similar binding interactions and/or binding surface in the large binding site.

The large binding site includes:
1. the known primary binding site for sialylated structures in human influenza hemagglutinin, the region of the large binding site is called here “the primary site” or “Region A” and
2. so called secondary sialic acid binding site on the surface of the hemagglutinin, wherein the sialic acid or surprisingly also certain other terminal monosaccharide residues or analogs thereof can be bound by novel binding mode, the region of the large binding site is called here “the secondary site” or “Region C” and
3. a groove-like region on surface of hemagglutinin bridging the primary and secondary sites, called here “the bridging site” or “Region B”.

### The Conserved Peptide Sequences of the Large Binding Site

Molecular modelling of mutated sites on the surface of influenza hemagglutinin revealed that many of amino acid residues on the large binding site are strongly conserved and part of the amino acid residues are semiconservatively conserved. The conservation of the protein structures further indicates the biological importance of the large binding site of the hemagglutinin. The virus cannot mutate nonconservatively the large binding site without losing its binding to the sialylated saccharide receptors on the target tissue. It clear that the large binding site is of special interest in design of novel medicines for influenza, which can stop the spreading of the virus.

### Conservation of the Large Binding Site Between Species

Furthermore, it was found out that the large binding sites in general are conserved between various influenza virus strains. Mutations were mapped from hemagglutinins from 100 strains closely related to strain X31. The large binding site was devoid of mutations or contained conservatively mutated amino acids in contrast to the surrounding regions. The large binding site recognized sialylated polylactosamines.

Animal hemagglutinins, especially avian hemagglutinins, are important because pandemic influenza strains has been known to have developed from animal hemagglutinins such as hemagglutinins from chicken or ducks. Also pigs are considered to have been involved in development of new influenza strains. The recognition of large carbohydrate structures on the surface of influenza hemagglutinin has allowed the evolution of the large binding site between terminal carbohydrate structures containing α3- and/or α6-linked sialic acids.

The pandemic strains of bird origin may be more α3-sialic acid specific, while the current human binding strains are more α6-specific. The present invention is further directed to mainly or partially α3-specific large binding sites. The present invention is further directed to substances to block the binding to mainly or partially α6-specific large binding sites.

### Design of Vaccines and Antibodies.

The large binding site and its conserved peptide sequences are of special interest in design of novel vaccines against influenza virus. The general problem with vaccines against influenza is that the virus mutates to immunity. A vaccine inducing the production of antibodies specific for the large binding site and its conserved peptide sequences will give general protection against various strains of influenza virus.

Furthermore, the invention is directed to the use of antibodies for blocking binding to the large binding site. Production of specific antibodies and human or humanized antibodies is known in the art. The antibodies, especially human or humanized antibodies, binding to the large binding site, are especially preferred for general treatment of influenza in human and analogously in animal.

Methods for producing peptide vaccines against influenza virus are well-known in the art. The present invention is specifically directed to selecting peptide epitopes for immunization and developing peptide vaccines comprising at least one one di- to decapeptide epitope, more preferably at least one tri- to hexapaptide epitope, and even more preferably at least one tri to pentapeptide epitope of the “large binding site” described by the invention in Table 1.

The peptide epitopes are preferably selected to contain the said peptide from among the important binding and/or conserved amino acids according to the Table 1, more preferably at least one peptide epitope is selected from region B. In another preferred embodiment two peptides are selected for immunization with two peptides so that at least one is from region B and one from region A or B. Preferably the peptide epitope is selected to comprise at least two conserved amino acid residues, in another preferred embodiments the peptide epitope is selected to comprise at least three conserved amino acid residues. In a preferred embodiment peptide epitope is modelled to be well accessible on the surface of the hemagglutinin protein.

### Combinations of Peptide Epitopes

It was realized that single peptide epitope has multiple strain specific variants. It would be useful to use several variants for current virus type for diagnostic and therapeutics according to the invention. The invention is especially directed to the use of the natural peptide sequences derived from the hemagglutinins, e.g. ones demonstrated in the Tables. The invention is further directed to use of multiple epitopes from different regions of the hemagglutinin large binding site in order to provide maximal immune recognition of virus by patients with different immune history against the viruses and different immune system, this was demonstrated with ELISA assay measuring varying reactions from several persons.

### The Complex Structure Between Large Polylactosamine Epitopes and the Large Binding Site

The invention is further directed to a substance including a complex of influenza virus hemagglutinin with a large polylactosamine epitope, called here “the complex structure”. The present invention is especially directed to the use of the complex structure for design of analogous substances with binding affinity towards hemagglutinin of influenza.

### The Specific Binding Interactions.

The present invention is directed to the use of the binding interactions observed between the large polylactosamine epitopes and the large binding site, called here “the specific binding interactions” for design of novel ligands for influenza virus hemagglutinin.

The invention showed that the binding of the influenza virus to the natural large poly-N-acetyllactosamines to the large binding site of the hemagglutinin could be inhibited by specific oligosacccharides. The present invention is directed to assay to be used for screening of substances binding to the large binding site. Preferably the assay comprises the large binding site, a carbohydrate conjugate or poly-N-acetyllactosamine ligand binding to the large binding site according to the invention and substances to be screened. The substances to be screened are screened for their ability to inhibit the binding between the large binding site and the saccharide according to the invention. The assay may be performed in solution by physical determination such as NMR-methods or fluorescence polarization, by labelling one of the compounds and using various solid phase assay wherein a non-labelled compound is immobilized on a solid phase and binding of alabelled compound is inhibited for example. The substances to be screened may be libraries of chemical synthesis, peptides, nucleotides, aptamers, antibodies etc.

### In Silico Screening

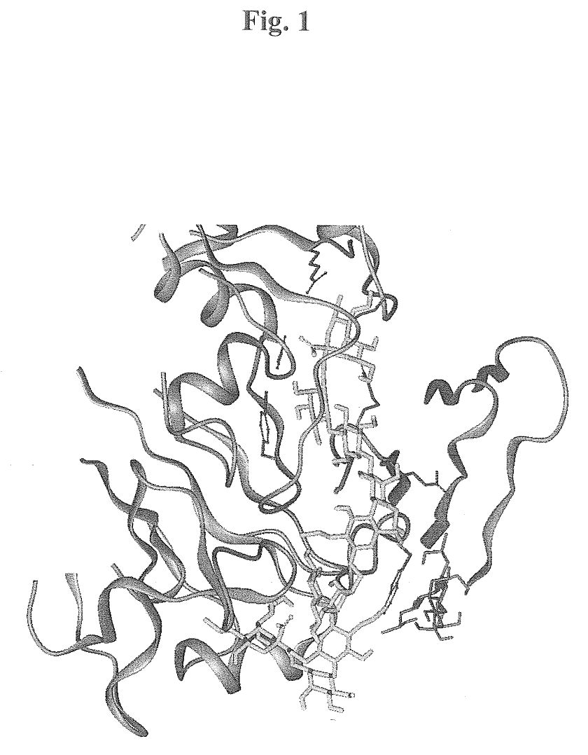

The three-dimensional structure of the large binding site of influenza hemagglutinin is defined by a set of structure coordinates as set forth in FIG. 1. The term “structure coordinates” refers to Cartesian coordinates derived from mathematical equations related to the patterns obtained on diffraction of a monochromatic beam of X-rays by the atoms (scattering centers) of the large binding site of influenza hemagglutinin in crystal form. The diffraction data are used to calculate an electron density map of the repeating unit of the crystal. The electron density maps are then used to establish the positions of the individual atoms of the large binding site of influenza hemagglutinin.

Those of skill in the art will understand that a set of structure coordinates for a protein or a protein-complex or a portion thereof, is a relative set of points that define a shape in three dimensions.

Thus, it is possible that an entirely different set of coordinates could define a similar or identical shape. Moreover, slight variations in the individual coordinates will have little effect on overall shape.

The variations in coordinates discussed above may be generated because of mathematical manipulations of the structure coordinates. For example, the structure coordinates set forth in FIG. 1 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three-dimensional shape is considered to be the same.

Various computational analyses are therefore necessary to determine whether a molecule or molecular complex or a portion thereof is sufficiently similar to all or parts of the large binding site of influenza hemagglutinin described above as to be considered the same.

Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, C alpha, C and O) for all conserved residues between the two structures being compared. We will also consider only rigid fitting operations.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex that has a root mean square deviation of conserved residue backbone atoms (N, C alpha, C, O) of less than 1.5 angstrom when superimposed on the relevant backbone atoms described by structure coordinates listed in FIG. 1 are considered identical. More preferably, the root mean square deviation is less than 1.0 angstrom.

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein or protein complex from the relevant portion of the backbone of the large binding site of influenza hemagglutinin as defined by the structure coordinates described herein.

Once the structure coordinates of a protein crystal have been determined they are useful in solving the structures of other crystals.

Thus, in accordance with the present invention, the structure coordinates of the large binding site of influenza hemagglutinin, and portions thereof is stored in a machine-readable storage medium. Such data may be used for a variety of purposes, such as drug discovery and x-ray crystallographic analysis or protein crystal.

Accordingly, in one embodiment of this invention is provided a machine-readable data storage medium comprising a data storage material encoded with the structure coordinates set forth in FIG. 1.

For the first time, the present invention permits the use of structure-based or rational drug design techniques to design, select, and synthesize chemical entities, including inhibitory compounds that are capable of binding to the large binding site of influenza hemagglutinin, or any portion thereof.

One particularly useful drug design technique enabled by this invention is iterative drug design. Iterative drug design is a method for optimizing associations between a protein and a compound by determining and evaluating the three-dimensional structures of successive sets of protein/compound complexes.

Those of skill in the art will realize that association of natural ligands or substrates with the binding pockets of their corresponding receptors or enzymes is the basis of many biological mechanisms of action. The term “binding site”, as used herein, refers to a region of a molecule or molecular complex, that, as a result of its shape, favorably associates with another chemical entity or compound. Similarly, many drugs exert their biological effects through association with the binding pockets of receptors and enzymes. Such associations may occur with all or any parts of the binding pockets. An understanding of such associations will help lead to the design of drugs having more favorable associations with their target receptor or enzyme, and thus, improved biological effects. Therefore, this information is valuable in designing potential ligands or inhibitors of receptors or enzymes, such as blockers of hemagglutinin.

The term “associating with” or “interacting with” refers to a condition of proximity between chemical entities or compounds, or portions thereof. The association or interaction may be non-covalent, wherein the juxtaposition is energetically favored by hydrogen bonding or van der Waals or electrostatic interactions, or it may be covalent.

In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex. Advantageously, the large binding site of influenza hemagglutinin crystals, may be soaked in the presence of a compound or compounds, such as hemagglutinin inhibitors, to provide hemagglutinin/ligand crystal complexes.

As used herein, the term “soaked” refers to a process in which the crystal is transferred to a solution containing the compound of interest.

### The Storage Medium

The storage medium in which the atomic co-ordinates are provided is preferably random access memory (RAM), but may also be read-only memory (ROM e.g. CDROM), or a diskette. The storage medium may be local to the computer, or may be remote (e.g. a networked storage medium, including the internet).

The invention also provides a computer-readable medium for a computer, characterised in that the medium contains atomic co-ordinates of the large binding site of influenza hemagglutinin.

The atomic co-ordinates are preferably those set forth in FIG. 1, or variants thereof.

Any suitable computer can be used in the present invention.

### Molecular Modelling Techniques

Molecular modelling techniques can be applied to the atomic co-ordinates of the large binding site of influenza hemagglutinin to derive a range of 3D models and to investigate the structure of ligand binding sites. A variety of molecular modelling methods are available to the skilled person for use according to the invention [e.g. ref 5].

At the simplest level, visual inspection of a computer model of the large binding site of influenza hemagglutinin can be used, in association with manual docking of models of functional groups into its binding sites.

Software for implementing molecular modelling techniques may also be used. Typical suites of software include CERIUS2 [Available from Molecular Simulations Inc], SYBYL [Available from Tripos Inc], AMBER [Available from Oxford Molecular], HYPERCHEM [Available from Hypercube Inc], INSIGHT II [Available from Molecular Simulations Inc], CATALYST [Available from Molecular Simulations Inc], CHEMSITE [Available from Pyramid Learning], QUANTA [Available from Molecular Simulations Inc]. These packages implement many different algorithms that may be used according to the invention (e.g. CHARMm molecular mechanics [Brooks et al. (1983) J. Comp. Chem. 4: 187-217]). Their uses in the methods of the invention include, but are not limited to: (a) interactive modelling of the structure with concurrent geometry optimisation (e.g. QUANTA); (b) molecular dynamics simulation of the large binding site of influenza hemagglutinin (e.g. CHARMM, AMBER); (c) normal mode dynamics simulation of the large binding site of influenza hemagglutinin (e.g. CHARMM).

Modelling may include one or more steps of energy minimisation with standard molecular mechanics force fields, such as those used in CHARMM and AMBER.

These molecular modelling techniques allow the construction of structural models that can be used for in silico drug design and modelling.

### Pharmacophore Searching

As well as using de novo design, a pharmacophore of the large binding site of influenza hemagglutinin can be defined i.e. a collection of chemical features and 3D constraints that expresses specific characteristics responsible for biological activity. The pharmacophore preferably includes surface-accessible features, more preferably including hydrogen bond donors and acceptors, charged/ionisable groups, and/or hydrophobic patches. These may be weighted depending on their relative importance in conferring activity.

Pharmacophores can be determined using software such as CATALYST (including HypoGen or HipHop) [Available from Molecular Simulations Inc], CERIUS2, or constructed by hand from a known conformation of a lead compound. The pharmacophore can be used to screen in silico compound libraries, using a program such as CATALYST [Available from Molecular Simulations Inc].

Suitable in silico libraries include the Available Chemical Directory (MDL Inc), the Derwent World Drug Index (WDI), BioByteMasterFile, the National Cancer Institute database (NCI), and the Maybridge catalog.

The term “treatment” used herein relates both to treatment in order to cure or alleviate a disease or a condition, and to treatment in order to prevent the development of a disease or a condition. The treatment may be either performed in an acute or in a chronic way.

The pharmaceutical composition according to the invention may also comprise other substances, such as an inert vehicle, or pharmaceutically acceptable carriers, preservatives etc., which are well known to persons skilled in the art.

The substance or pharmaceutical composition according to the invention may be administered in any suitable way, although an oral or nasal administration especially in the form of a spray or inhalation are preferred. The nasal and oral inhalation and spray dosage technologies are well-known in the art. The preferred dose depend on the substance and the infecting virus. In general dosages between 0.01 mg and 500 mg are preferred, more preferably the dose is between 0.1 mg and 50 mg. The dose is preferably administered at least once daily, more preferably twice per day and most preferably three or four times a day. In case of excessive secretion of mucus and sneezing or cough the dosage may be increased with 1-3 doses a day.

The present invention is directed to novel divalent molecules as substances. Preferred substances includes preferred molecules comprising the flexible spacer structures and peptide and/or oxime linkages. The present invention is further directed to the novel uses of the molecules as medicines. The present invention is further directed to in methods of treatments applying the substances according to the invention.

The term “patient”, as used herein, relates to any human or non-human mammal in need of treatment according to the invention.

Glyco lipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, and 131-6 can be shortened as α3, β3, β4, and β6, respectively. Lactosamine refers to N-acetyllactosamine, Galβ4GlcNAc, and sialic acid is N-acetylneuraminic acid (Neu5Ac, NeuNAc or NeuAc) or N-glycolylneuraminic acid (Neu5Gc) or any other natural sialic acid. Term glycan means here broadly oligosaccharide or polysaccharide chains present in human or animal glycoconjugates, especially on glyco lipids or glycoproteins. In the shorthand nomenclature for fatty acids and bases, the number before the colon refers to the carbon chain length and the number after the colon gives the total number of double bonds in the hydrocarbon chain.

### Antibody Substances

Every method of using antibody substances of the invention, whether for therapeutic, diagnostic, or research purposes, is another aspect of the invention. For example, the invention further contemplates use of the peptide motifs as a method for screening for antibody substances. One aspect the invention provides a method of screening an antibody substance for peptide motif or peptide motifs and influenza virus neutralization activity comprising: contacting a peptide motif/antigen and influenza virus in the presence and absence of an antibody substance; and measuring binding between the peptide motif/antigen and the virus in the presence and absence of the antibody substance, wherein reduced binding in the presence of the antibody substance indicates virus neutralization activity for the antibody substance; wherein the peptide motif/antigen comprises at least one member selected from the group consisting of KVR, KVN, WVR, TPNPENGT, KANPANDL, VTKGVSAS, GGSNA, and EASSGVSSA region; and combinations thereof; wherein the virus is at least one member selected from the group consisting of H1, H2, H3, H4 or H5 HA subtype of the influenza virus A; and wherein the antibody substance comprises an antibody substance according to the invention.

For example, one aspect of the invention is a method for inhibiting, preventing or alleviating influenza virus caused symptoms, by vaccination, comprising administering to a mammalian subject in need of inhibition, prevention or alleviation of influenza virus caused symptoms a peptide motif or peptide motifs according to the invention, in an amount effective to inhibit, alleviate or prevent influenza virus caused symptoms. Methods to determine the extent of inhibition, prevention and alleviation influenza virus caused symptoms are described herein.

For example, one aspect of the invention is a method for inhibiting, preventing or alleviating influenza virus caused symptoms comprising administering to a mammalian subject in need of inhibition, prevention or alleviation of influenza virus caused symptoms an antibody substance according to the invention, in an amount effective to inhibit, alleviate or prevent influenza virus caused symptoms. Methods to determine the extent of inhibition, prevention and alleviation influenza virus caused symptoms are described herein.

The invention further provides a method of inhibiting, preventing or alleviating influenza virus caused symptoms comprising steps of: (a) determining peptide motifs and/or region composition of an influenza virus from a sample or a mammalian subject, (b) assaying binding between peptide motifs and the antibody substances; and (c) administering to a subject an antibody substance according to the invention, wherein the antibody substance binds to peptide motif(s) identified in step (a).

The invention further provides a method of inhibiting, preventing or alleviating influenza virus caused symptoms comprising steps of: (a) determining peptide motifs and/or region composition of an influenza virus from a sample or a mammalian subject, (b) administering to a subject peptide motif(s) according to the invention.

Antibody substances of the invention are useful for preventing, alleviating and/or inhibiting influenza causes symptoms. The invention provides antibody substances for administration to human beings (e.g., monoclonal and polyclonal antibodies, single chain antibodies, chimeric antibodies, bifunctional/bispecific antibodies, humanized antibodies, human antibodies, and complementarity determining region (CDR)-grafted antibodies, including compounds which include CDR sequences which specifically recognize a polypeptide of the invention) specific for polypeptides of interest to the invention. Preferred antibodies are human antibodies which are produced and identified according to methods described in WO 93/11236, published Jun. 20, 1993, which is incorporated herein by reference in its entirety. Antibody fragments, including Fab, Fab′, F(ab′)₂, Fv, and single chain antibodies (scFv) are also provided by the invention. Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lyso lecithin, pluronic polyols, polyanions, oil emulsions, keyhole limpet hemocyanins, dinitrophenol, and potentially useful human adjuvants such as BCG {Bacille Calmette-Guerin) and Corynebacterium parvum.

A monoclonal antibody to a peptide motif(s) may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Agl4, FO, NSO/U, MPC-I 1, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81 : 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

### Nucleic Acids of the Invention

RNA viruses, including the influenza A virus, tend to have high mutation rates due to the low fidelity nature of RNA replication when compared to DNA replication. As a result, influenza viruses tend to evolve rapidly. Furthermore, influenza A viruses tend to undergo genetic reassortment between viral strains, which mechanism has contributed to the development of the various HA and NA subtypes. The inventors compared the sequence of the hemagglutinin (“HA”) gene from known influenza A sequences. Surprisingly, despite the high mutation rate within influenza viruses, the inventors have discovered short regions of highly conserved sequences unique to all subtypes, which regions are suitable to identify or detect the presence of influenza A and/or a subtypes or subtypes in a sample.

The sequences used in the comparison were obtained from publicly available databases and were compared using a variety of sequence comparison software Influenza Virus Resource.

These sequence comparisons allowed the inventors to develop forward and reverse primers set out in Table 1, directed to conserved regions of the HA gene of influenza virus subtypes H1, H3 and H5, for use in a detection assay, for example, reverse-transcription followed by polymerase chain reaction amplification (“RT-PCR”). The comparison of such a large number of viruses allowed for the design of primers directed to well-conserved regions of the HA gene, thus targeting regions that are less likely to be affected by mutational changes and thereby providing primers that can detect a larger pool of H variants than primers that are currently available.

The term “isolate” as used herein refers to a particular virus or clonal population of virus particles, isolated from a particular biological source, such as a patient, which has a particular genetic sequence. Different isolates may vary at only one or several nucleotides, and may still fall within the same viral subtype. A viral subtype refers to any of the subtypes of HA classified according to the antigenicity of these glycoproteins.

The inventors found that in certain conserved regions, one or more nucleotides at a specific location vary between isolates. For those regions, a family of primers can be developed, each primer within the family being based on a conserved sequence of the HA gene, but varying at one or more particular bases within the conserved sequence.

As will be understood by a skilled person, a “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the primer and target molecule and the degree of stringency of the hybridization conditions. The degree of hybridization stringency is affected by parameters such as the temperature, salt concentration, and concentration of organic molecules, such as formamide, and may be determined using methods that are known to those skilled in the art. Primers can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), nucleic acid microarrays, and other methods that are known to those skilled in the art.

The term “RNA” refers to a sequence of two or more covalently bonded, naturally occurring or modified ribonucleotides. The RNA may be single stranded or double stranded. The term “DNA” refers to a sequence of two or more covalently bonded, naturally occurring or modified deoxyribonucleotides, including cDNA and synthetic (e.g., chemically synthesized) DNA, and may be double stranded or single stranded. By “reverse transcribed DNA” or “DNA reverse transcribed from” is meant complementary or copy DNA (cDNA) produced from an RNA template by the action of RNA-dependent DNA polymerase (reverse transcriptase).

Influenza A virus is a single stranded RNA virus and in some embodiments, the primer has a DNA sequence that corresponds to the RNA sequence of a conserved region of the HA gene of human, avian and/or swine influenza virus subtype H1-5. Such primers may be used as a forward primer when sequencing or amplifying DNA reverse transcribed from the HA genes.

TABLE 1

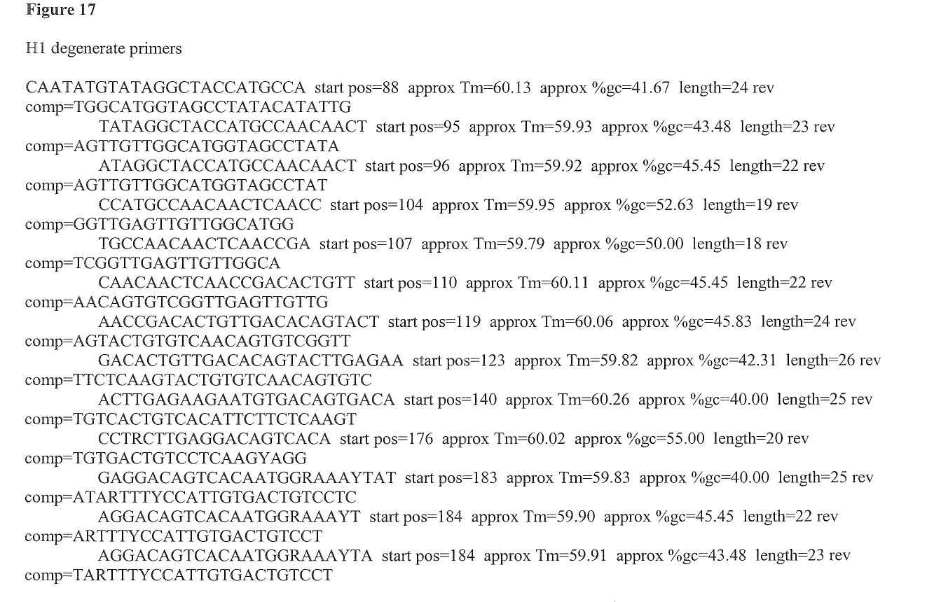

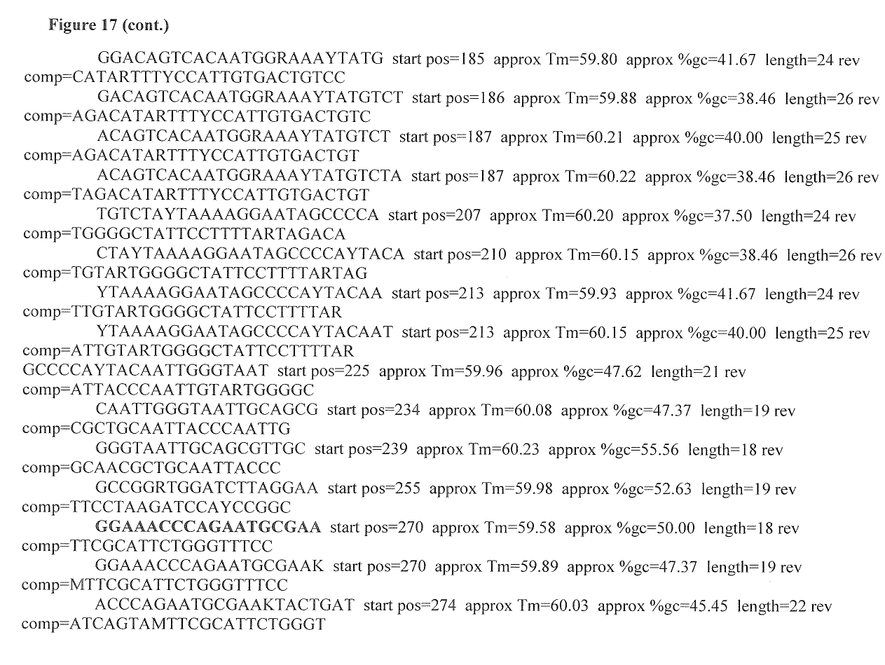

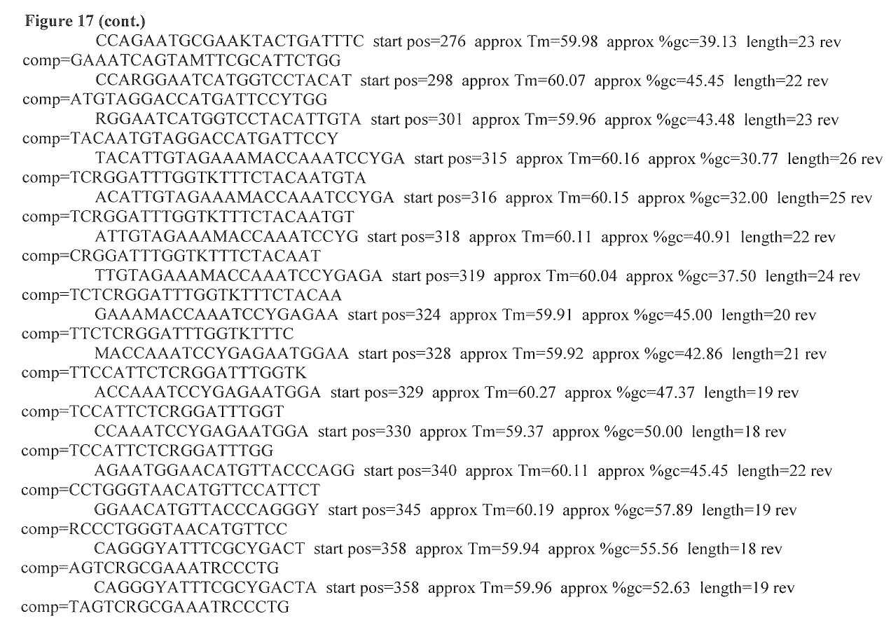

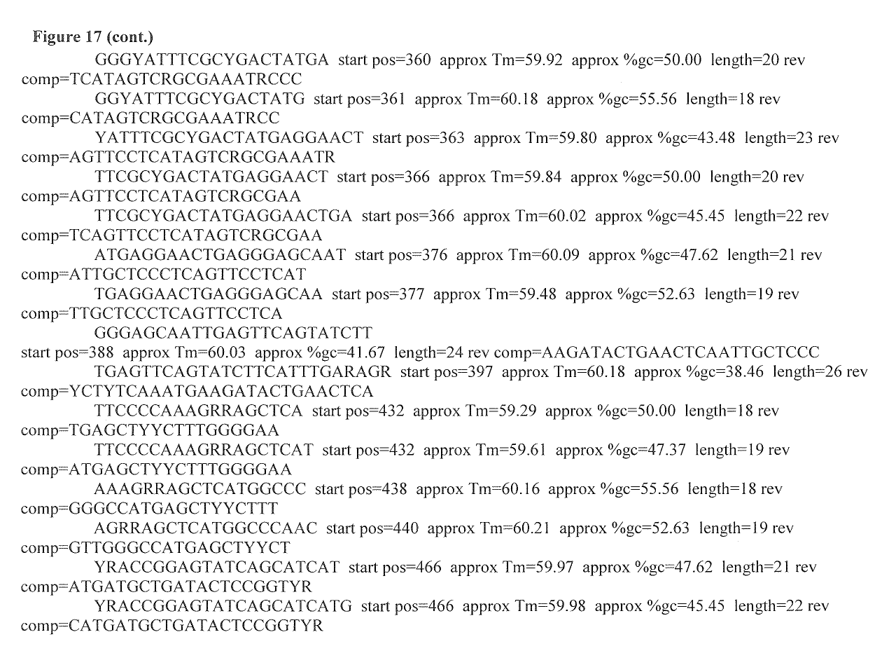

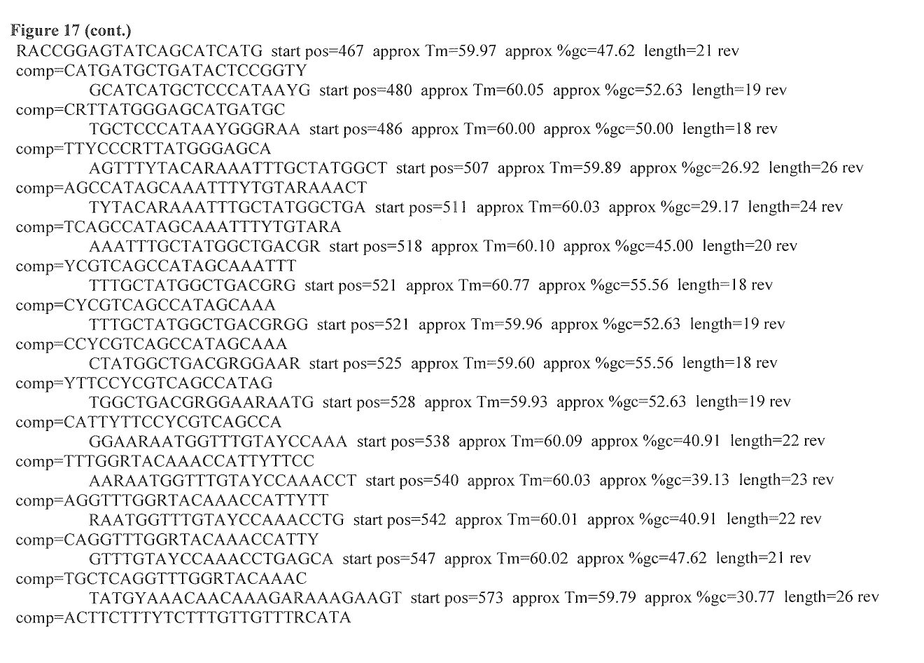

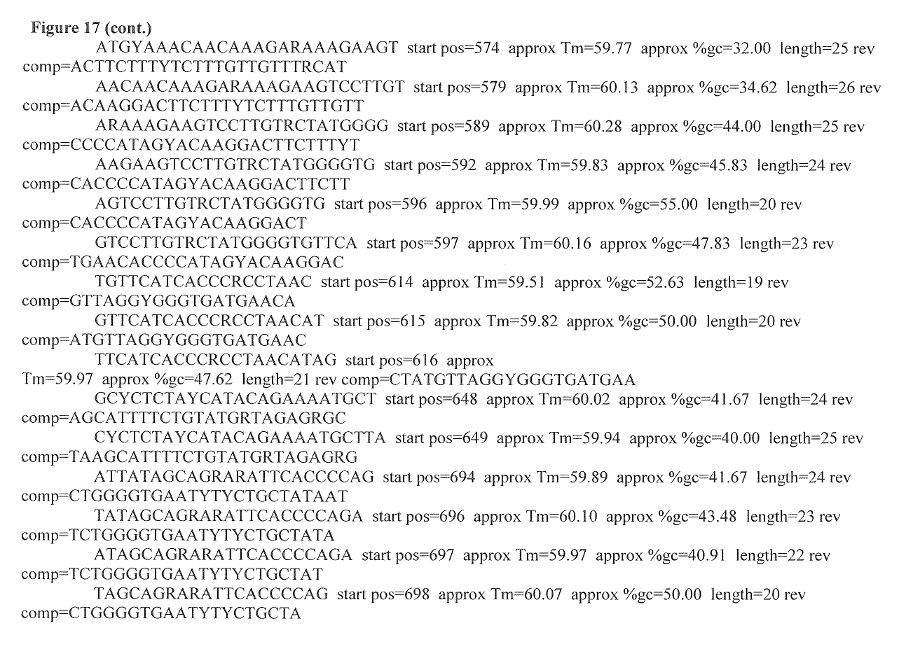

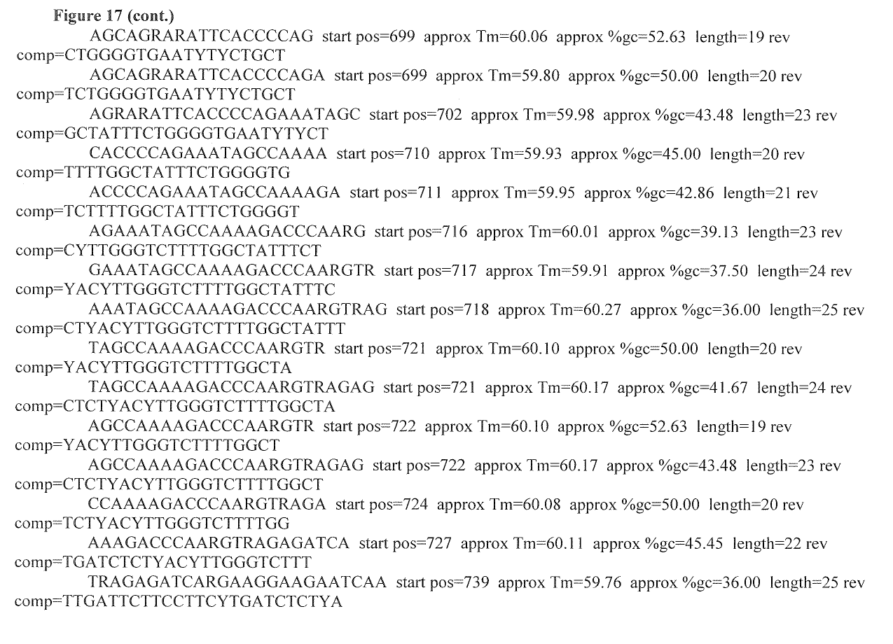

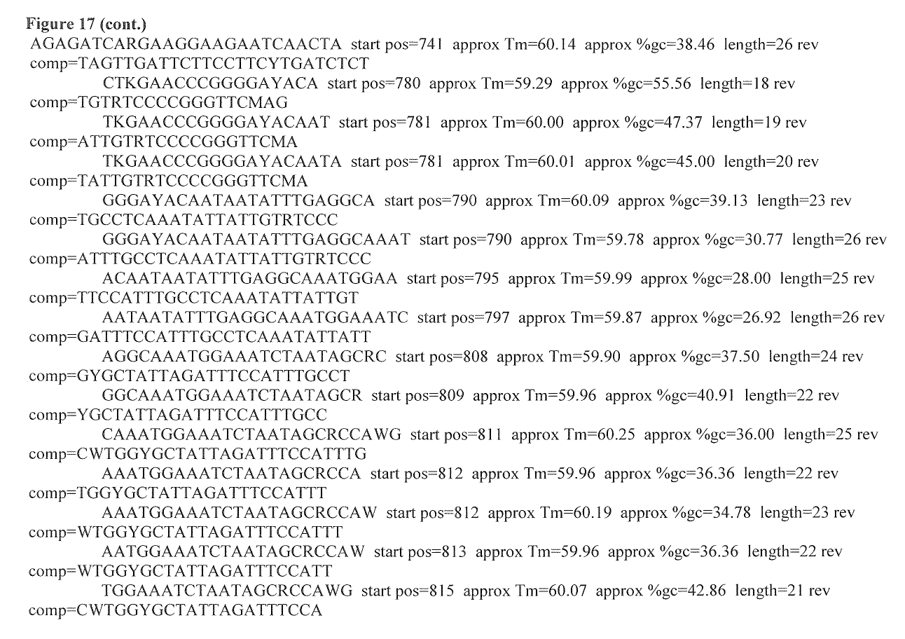

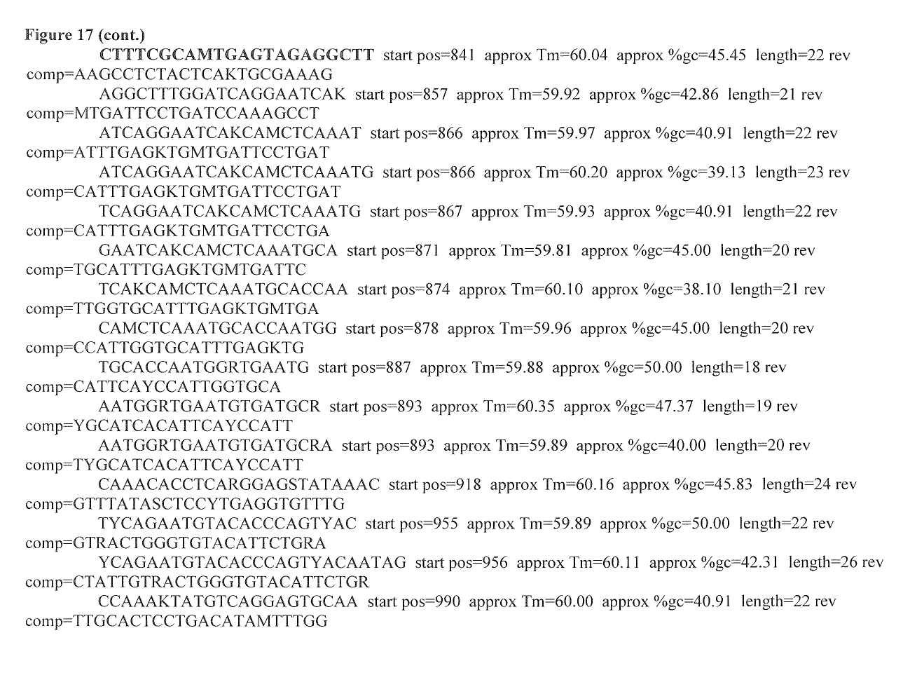

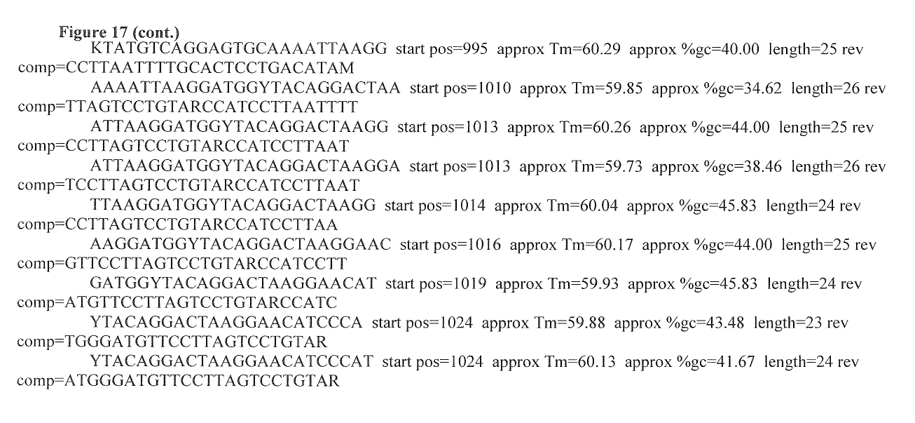

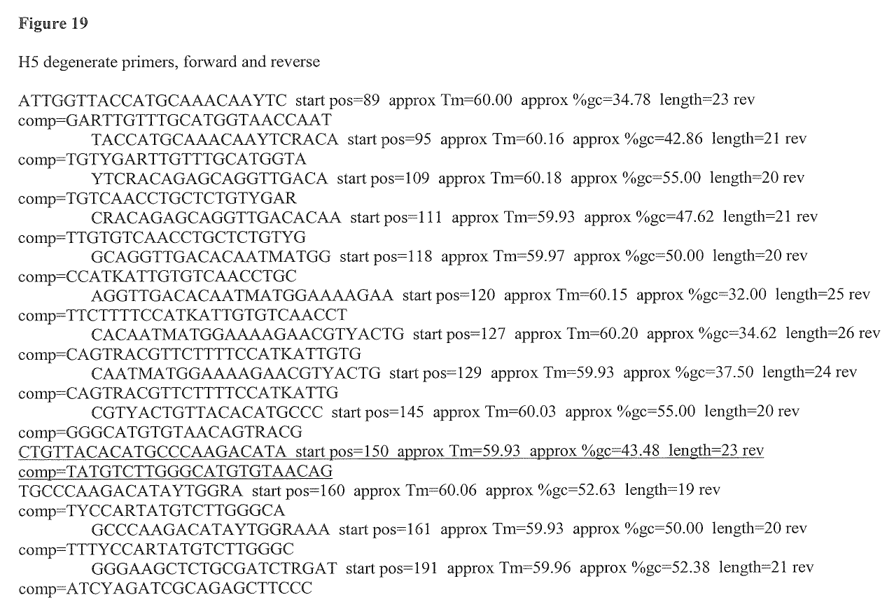

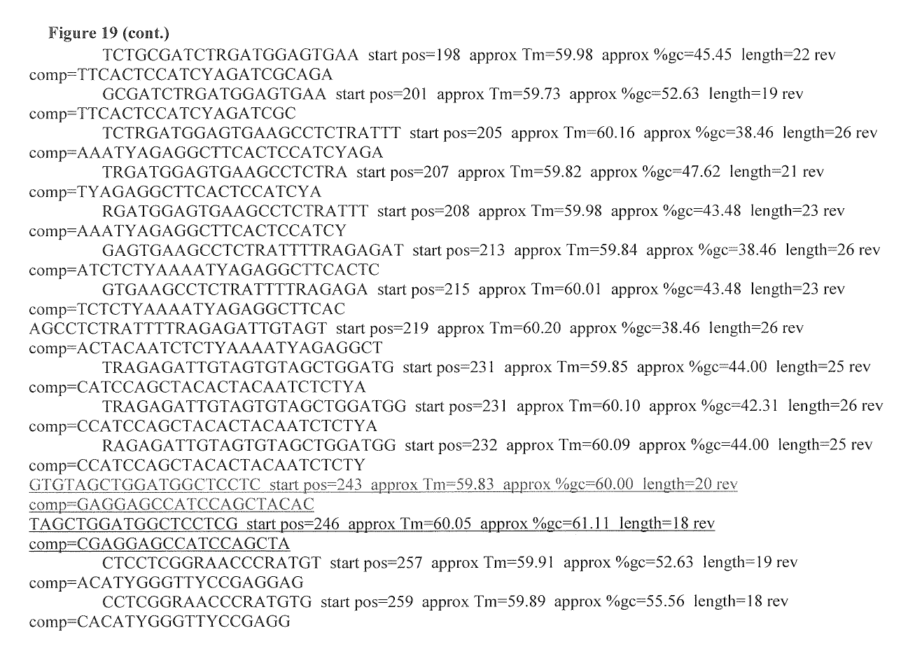

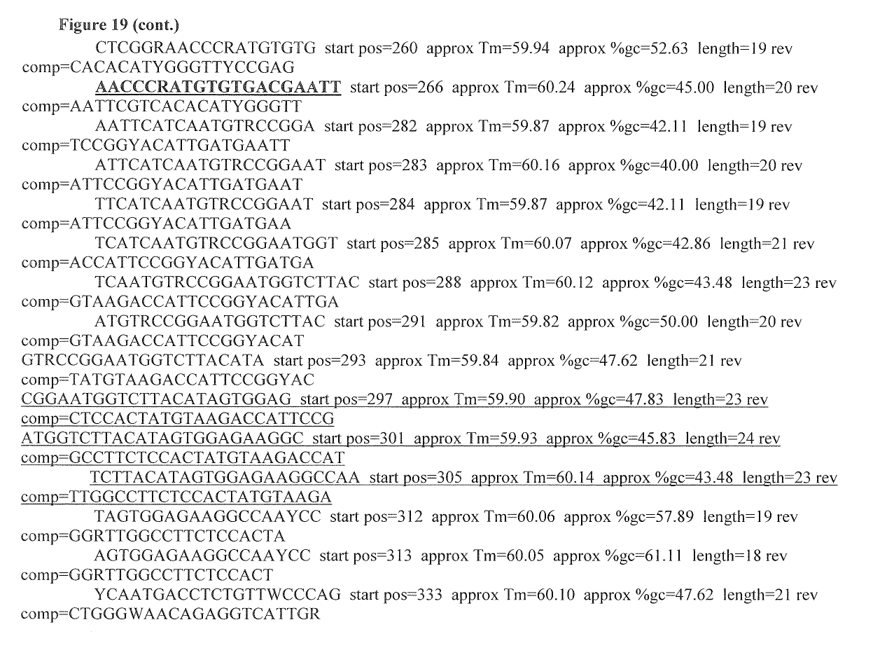

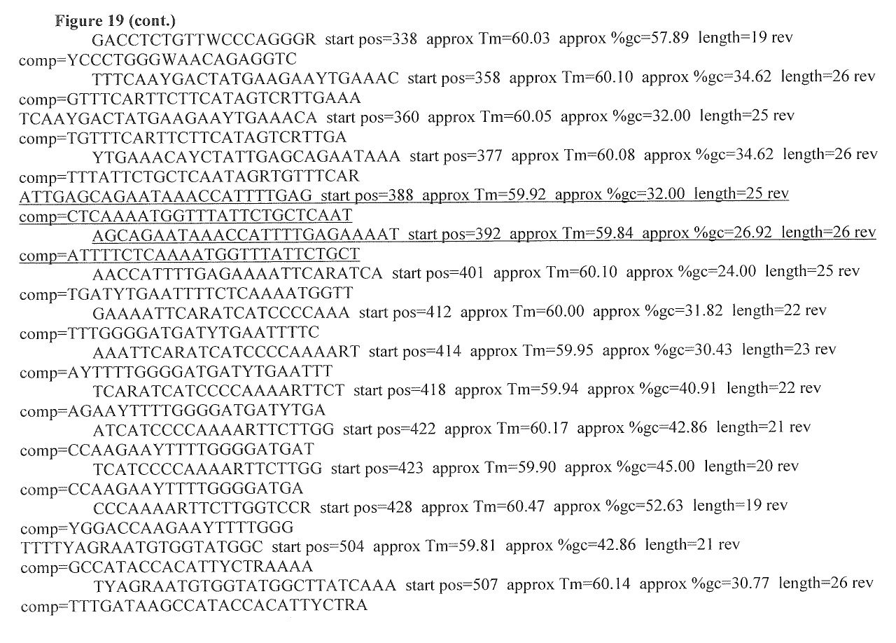

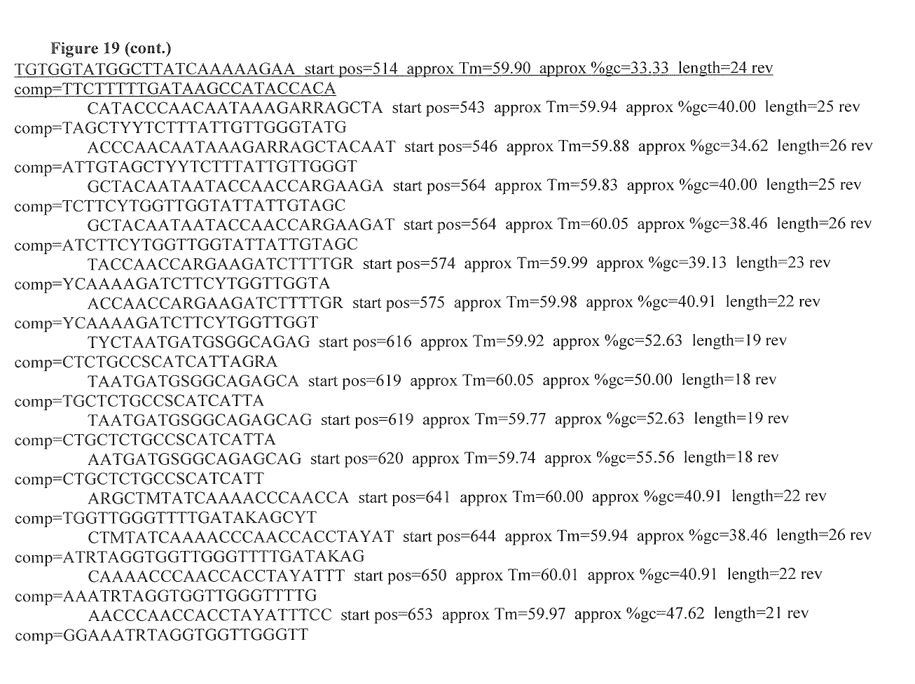

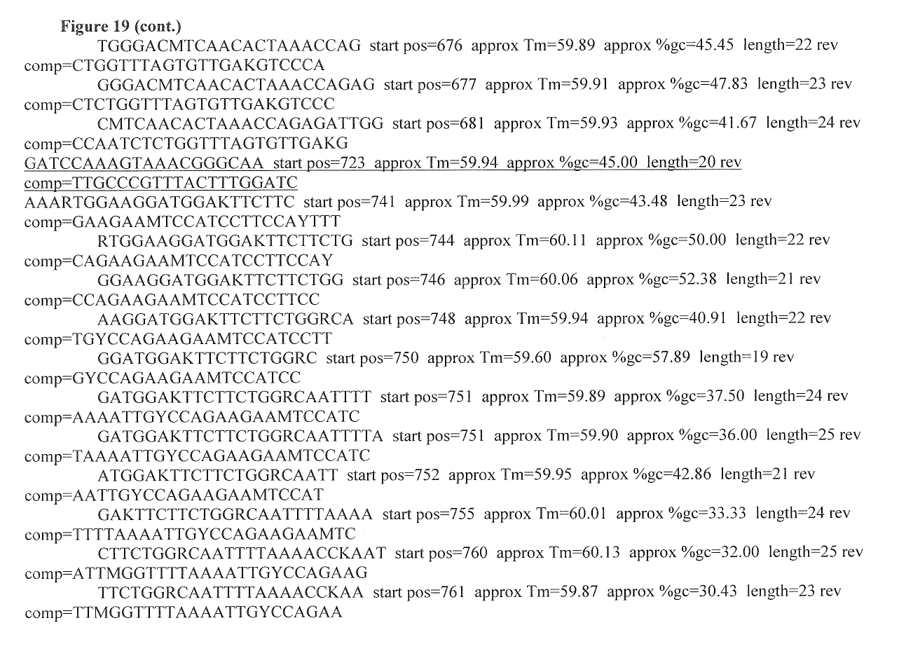

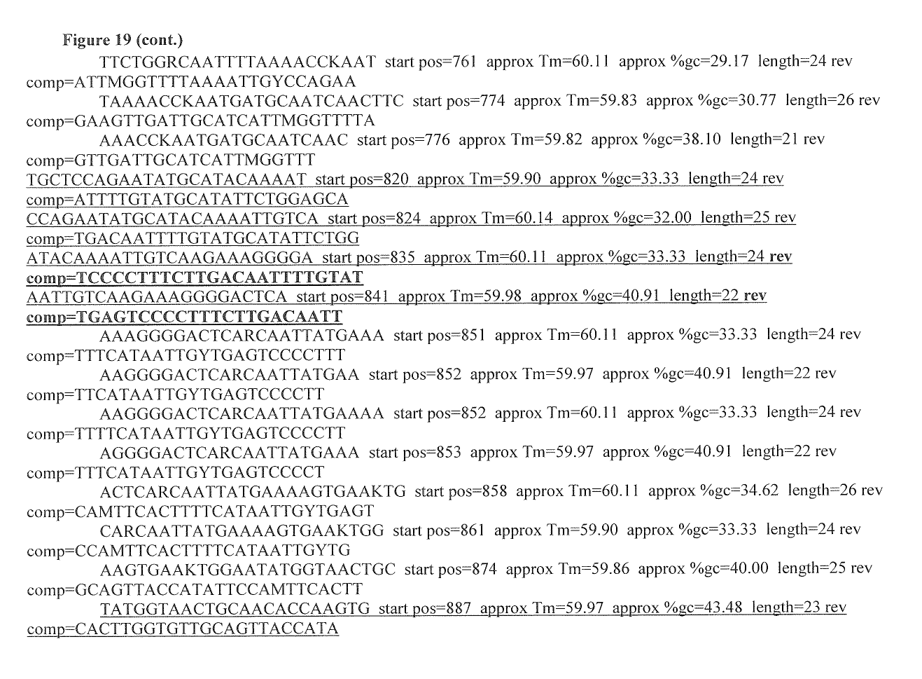

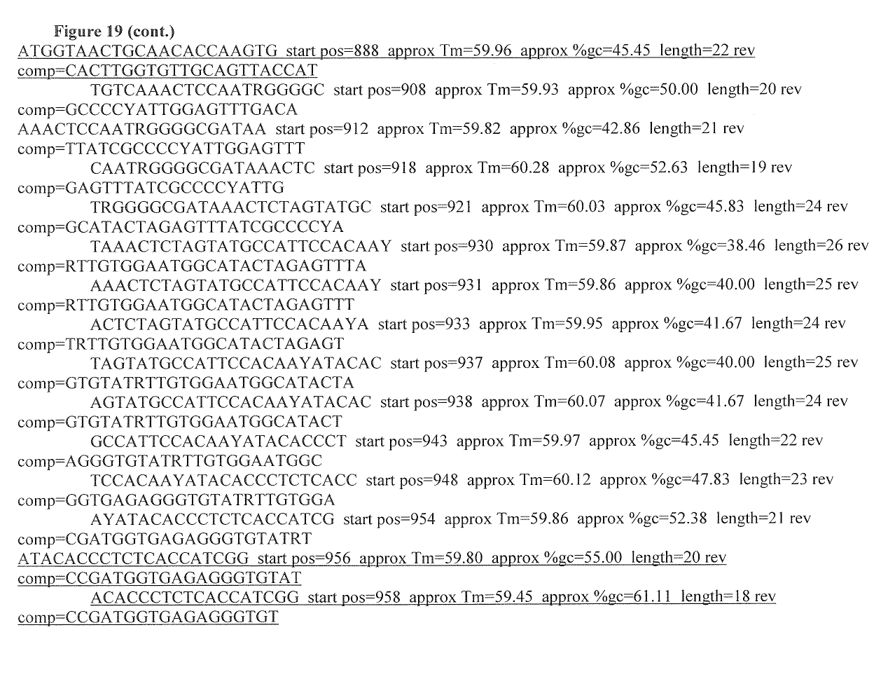

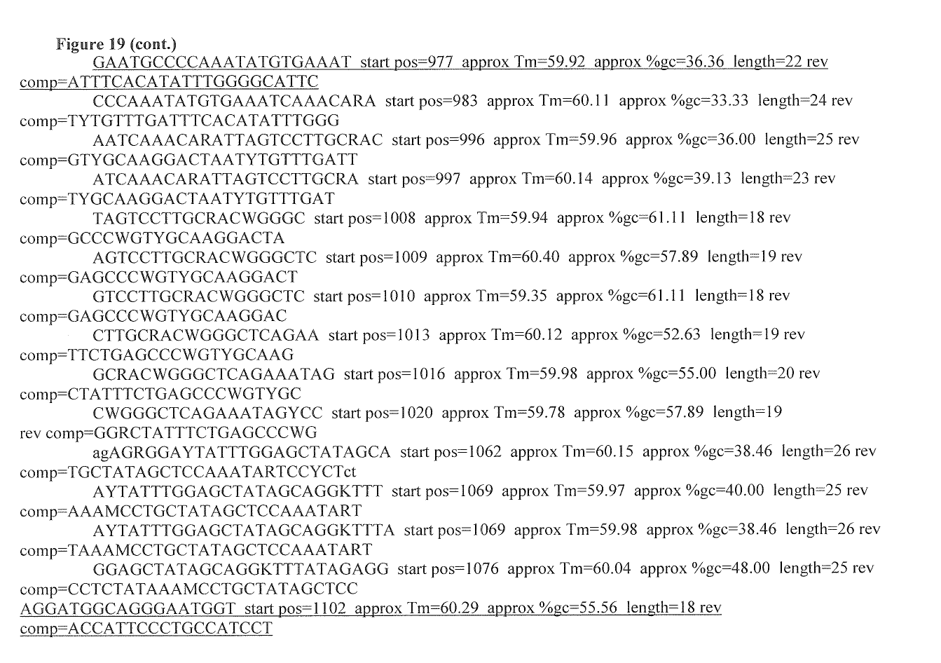

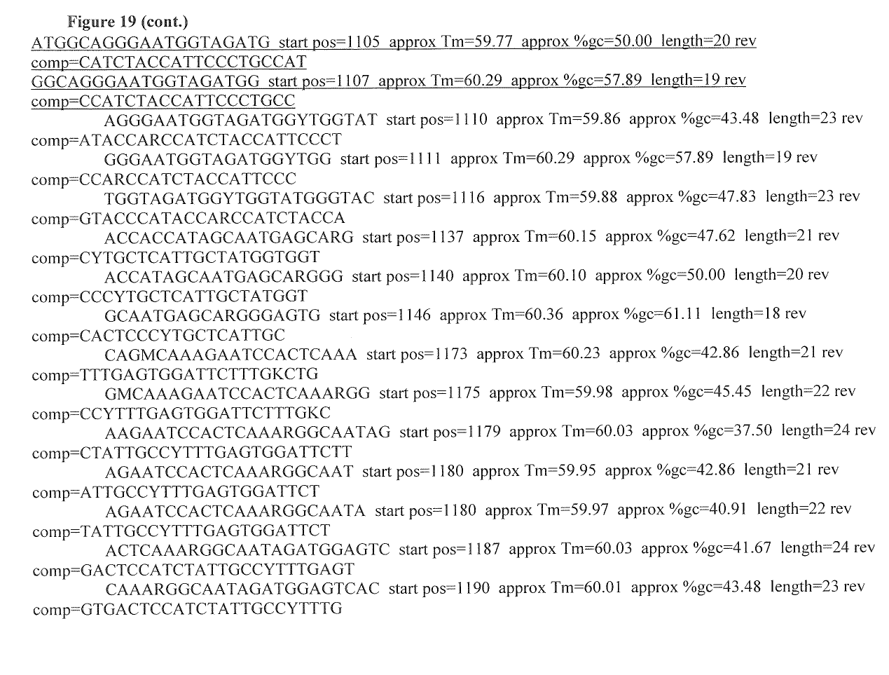

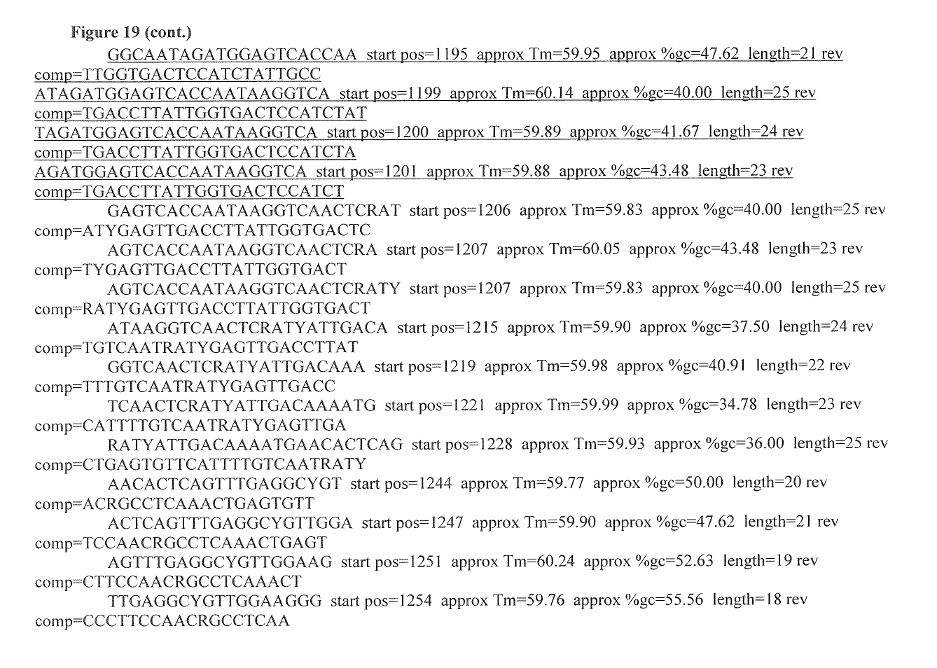

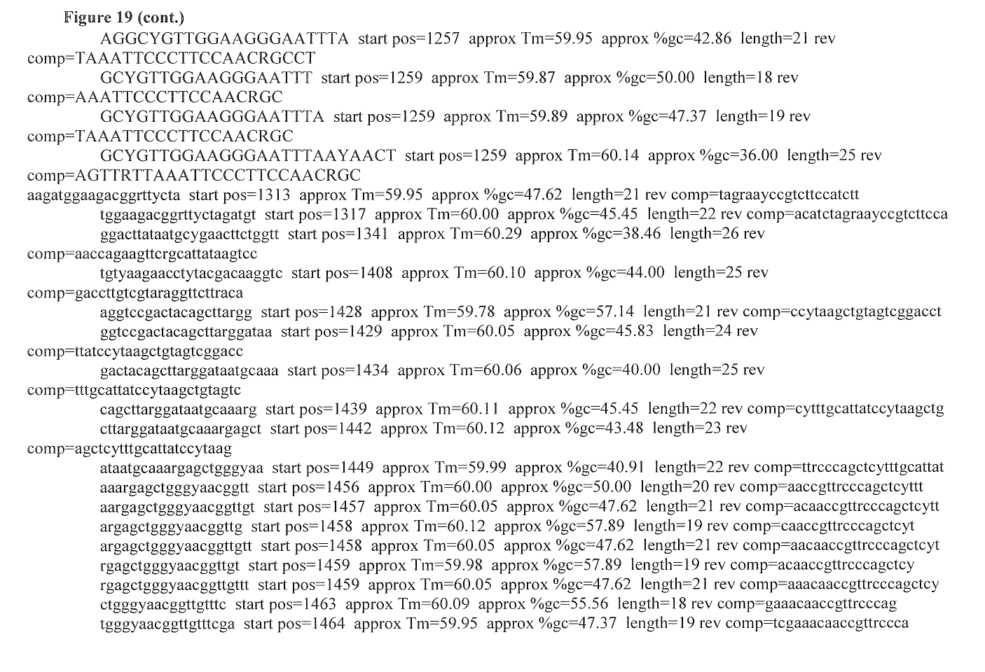

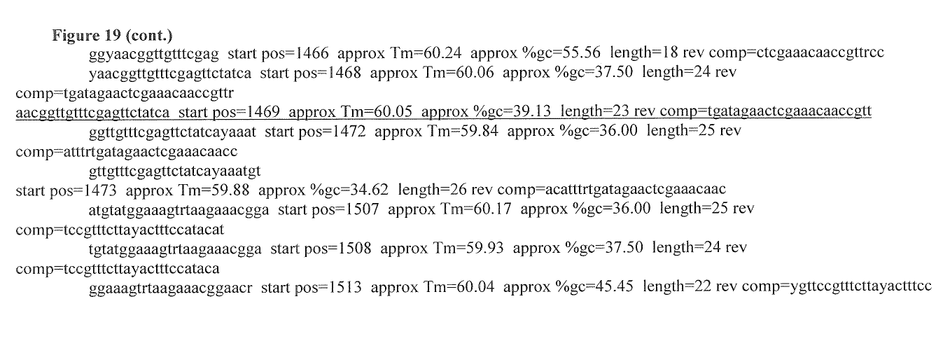

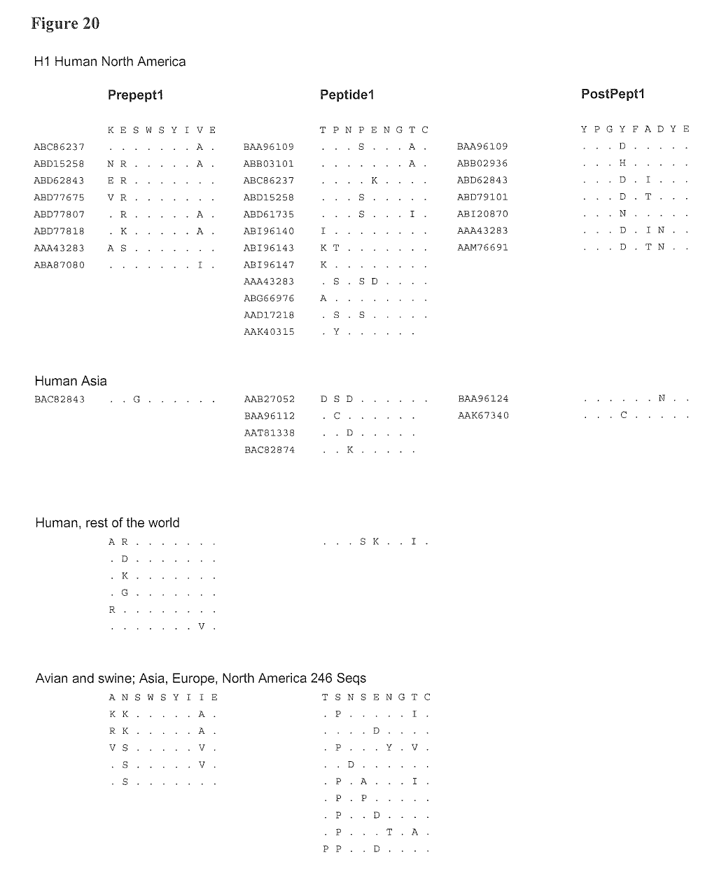

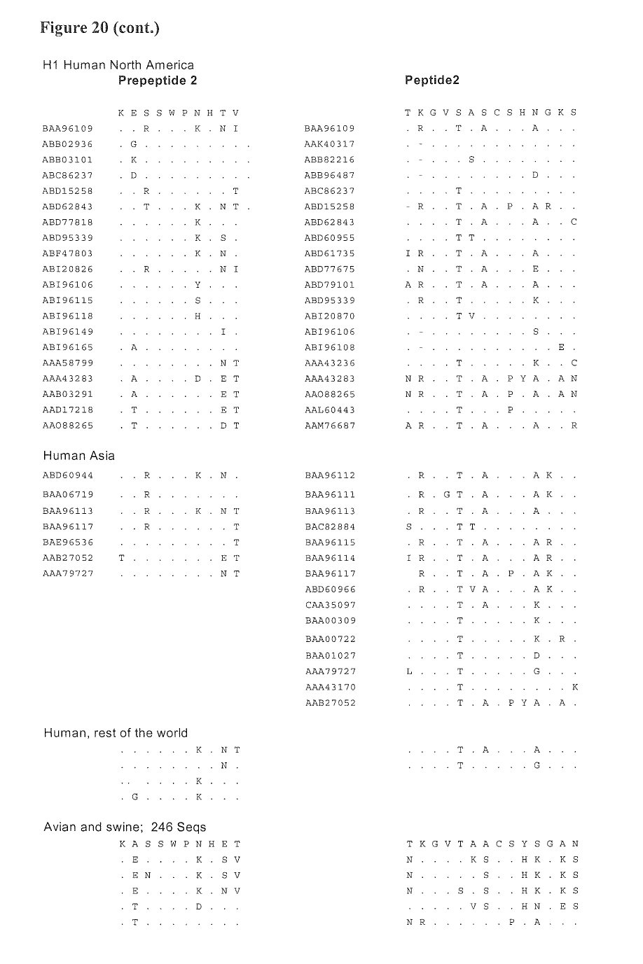

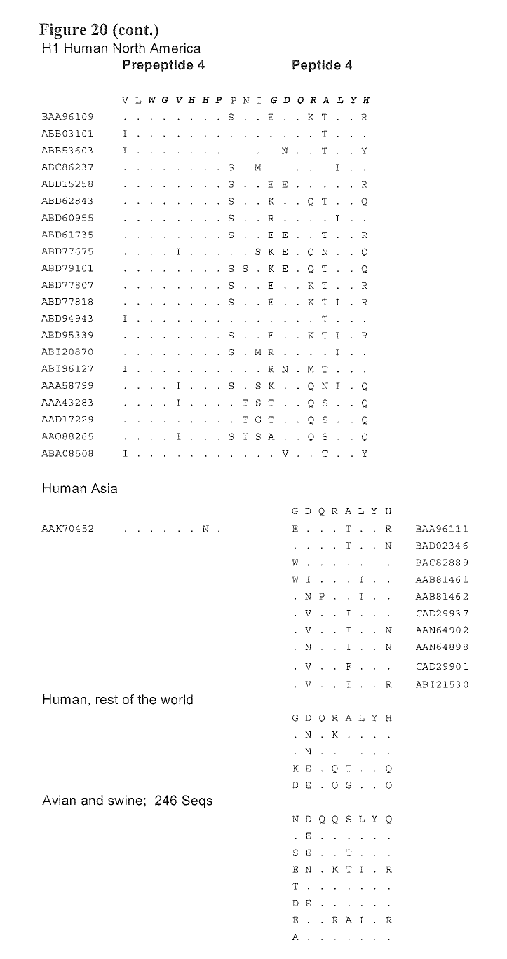

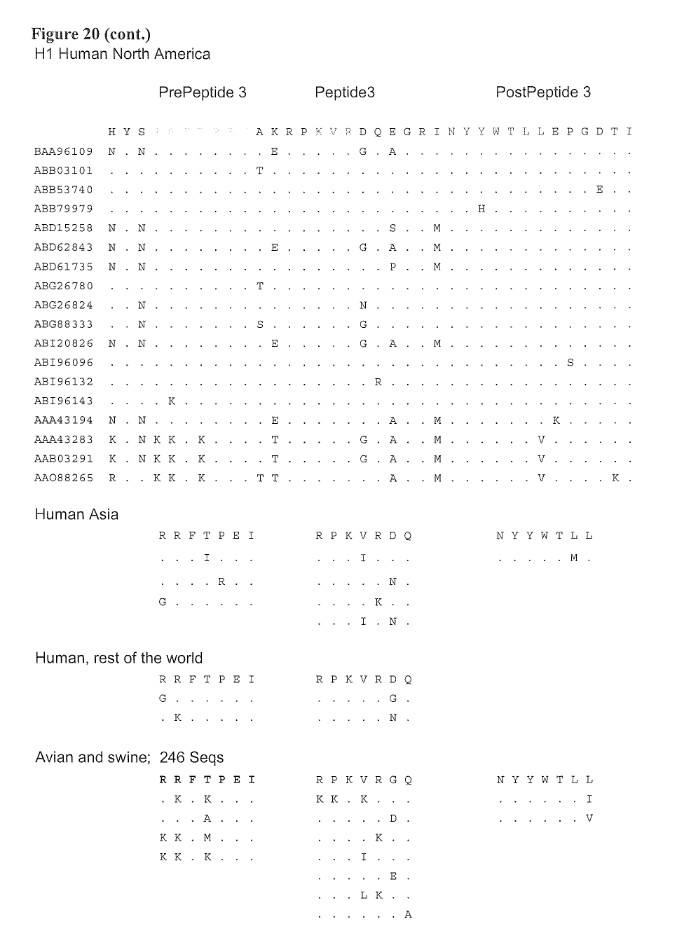

Forward and reverse primers for the H1 gene. Deg denotes
degeneracy of a primer. ID F denotes forward primer and ID R
reverse primer for the complementary sequence. Additional
primers can be found at FIGS. 17-19.

| | | | |
|---|---|---|---|
| ID F | | | ID R |
| No: | Deg | H1 Primer | No: |

| | | | |
|---|---|---|---|
| 1 | 0 | CAATATGTATAGGCTACCATGCCA start pos = 88 approx | |
| | | Tm = 60.13 approx % gc = 41.67 | |
| 2 | 0 | TATAGGCTACCATGCCAACAACT start pos = 95 approx | |
| | | Tm = 59.93 approx % gc = 43.48 | |
| 3 | 0 | ATAGGCTACCATGCCAACAACT start pos = 96 approx Tm = 59.92 | |
| | | approx % gc = 45.45 | |
| 4 | 0 | CCATGCCAACAACTCAACC start pos = 104 approx Tm = 59.95 | |
| | | approx % gc = 52.63 | |
| 5 | 0 | TGCCAACAACTCAACCGA start pos = 107 approx Tm = 59.79 | |
| | | approx % gc = 50.00 | |
| 6 | 0 | CAACAACTCAACCGACACTGTT start pos = 110 approx | |
| | | Tm = 60.11 approx % gc = 45.45 | |
| 7 | 0 | AACCGACACTGTTGACACAGTACT start pos = 119 approx | |
| | | Tm = 60.06 approx % gc = 45.83 | |
| 8 | 0 | GACACTGTTGACACAGTACTTGAGAA start pos = 123 approx | |
| | | Tm = 59.82 approx % gc = 42.31 | |
| 9 | 0 | ACTTGAGAAGAATGTGACAGTGACA start pos = 140 approx | |
| | | Tm = 60.26 approx % gc = 40.00 | |
| 10 | 0 | CAATTGGGTAATTGCAGCG start pos = 234 approx Tm = 60.08 | |
| | | approx % gc = 47.37 | |
| 11 | 0 | GGGTAATTGCAGCGTTGC start pos = 239 approx Tm = 60.23 | |
| | | approx % gc = 55.56 | |
| 12 | 0 | GGAAACCCAGAATGCGAA start pos = 270 approx Tm = 59.58 | |
| | | approx % gc = 50.00 | |
| 13 | 0 | AGAATGGAACATGTTACCCAGG start pos = 340 approx | |
| | | Tm = 60.11 approx % gc = 45.45 | |
| 14 | 0 | ATGAGGAACTGAGGGAGCAAT start pos = 376 approx Tm = 60.09 | |
| | | approx % gc = 47.62 | |
| 15 | 0 | TGAGGAACTGAGGGAGCAA start pos = 377 approx Tm = 59.48 | |
| | | approx % gc = 52.63 | |
| 16 | 0 | GGGAGCAATTGAGTTCAGTATCTT start pos = 388 approx | |
| | | Tm = 60.03 approx % gc = 41.67 | |
| 17 | 0 | CACCCCAGAAATAGCCAAAA start pos = 710 approx Tm = 59.93 | |
| | | approx % gc = 45.00 length = 20 | |
| | 0 | rev comp = TTTTGGCTATTTCTGGGGTG | 61 |
| 18 | 0 | ACCCCAGAAATAGCCAAAAGA start pos = 711 approx Tm = 59.95 | |
| | | approx % gc = 42.86 length = 21 | |
| | | rev comp = TCTTTTGGCTATTTCTGGGGT | 62 |
| 19 | 0 | ACAATAATATTTGAGGCAAATGGAA start pos = 795 approx | |
| | | Tm = 59.99 approx % gc = 28.00 length = 25 | |
| | | rev comp = TTCCATTTGCCTCAAATATTATTGT | 63 |
| 20 | 0 | AATAATATTTGAGGCAAATGGAAATC start pos = 797 approx | |
| | | Tm = 59.87 approx % gc = 26.92 length = 26 | |
| | | rev comp = GATTTCCATTTGCCTCAAATATTATT | 64 |
| 21 | 1-2 | CCTRCTTGAGGACAGTCACA start pos = 176 approx Tm = 60.02 | |
| | | approx % gc = 55.00 | |
| 22 | 1-2 | GAGGACAGTCACAATGGRAAAYTAT start pos = 183 approx | |
| | | Tm = 59.83 approx % gc = 40.00 | |
| 23 | 1-2 | AGGACAGTCACAATGGRAAAYT start pos = 184 approx | |
| | | Tm = 59.90 approx % gc = 45.45 | |
| 24 | 1-2 | GACAGTCACAATGGRAAAYTATGTCT start pos = 186 approx | |
| | | Tm = 59.88 approx % gc = 38.46 | |
| 25 | 1-2 | TGTCTAYTAAAAGGAATAGCCCCA start pos = 207 approx | |
| | | Tm = 60.20 approx % gc = 37.50 | |
| 26 | 1-2 | CTAYTAAAAGGAATAGCCCCAYTACA start pos = 210 approx | |
| | | Tm = 60.15 approx % gc = 38.46 | |
| 27 | 1-2 | GCCCCAYTACAATTGGGTAAT start pos = 225 approx Tm = 59.96 | |
| | | approx % gc = 47.62 | |
| 28 | 1-2 | GCCGGRTGGATCTTAGGAA start pos = 255 approx Tm = 59.98 | |
| | | approx % gc = 52.63 | |
| 29 | 1-2 | ACCCAGAATGCGAAKTACTGAT start pos = 274 approx | |
| | | Tm = 60.03 approx % gc = 45.45 | |
| 30 | 1-2 | CCARGGAATCATGGTCCTACAT start pos = 298 approx | |
| | | Tm = 60.07 approx % gc = 45.45 | |
| 31 | 1-2 | TACATTGTAGAAAMACCAAATCCYGA start pos = 315 approx | |
| | | Tm = 60.16 approx % gc = 30.77 | |
| 32 | 1-2 | TTGTAGAAAMACCAAATCCYGAGA start pos = 319 approx | |
| | | Tm = 60.04 approx % gc = 37.50 | |
| 33 | 1-2 | GAAAMACCAAATCCYGAGAA start pos = 324 approx Tm = 59.91 | |
| | | approx % gc = 45.00 | |
| 34 | 1-2 | ACCAAATCCYGAGAATGGA start pos = 329 approx Tm = 60.27 | |
| | | approx % gc = 47.37 | |
| 35 | 1-2 | GGAACATGTTACCCAGGGY start pos = 345 approx Tm = 60.19 | |
| | | approx % gc = 57.89 length = 19 | |
| | 1-2 | rev comp = RCCCTGGGTAACATGTTCC | 65 |
| 36 | 1-2 | CAGGGYATTTCGCYGACTA start pos = 358 approx Tm = 59.96 | |
| | | approx % gc = 52.63 length = 19 | |
| | 1-2 | rev comp = TAGTCRGCGAAATRCCCTG | 66 |
| 37 | 1-2 | TTCGCYGACTATGAGGAACT start pos = 366 approx Tm = 59.84 | |
| | | approx % gc = 50.00 length = 20 | |
| | | rev comp = AGTTCCTCATAGTCRGCGAA | 67 |
| 38 | 1-2 | TGAGTTCAGTATCTTCATTTGARAGR start pos = 397 approx | |
| | | Tm = 60.18 approx % gc = 38.46 length = 26 | |
| | 1-2 | rev comp = YCTYTCAAATGAAGATACTGAACTCA | 68 |
| 39 | 1-2 | TTCCCCAAAGRRAGCTCAT start pos = 432 approx Tm = 59.61 | |
| | | approx % gc = 47.37 length = 19 | |
| | 1-2 | rev comp = ATGAGCTYYCTTTGGGGAA | 69 |
| 40 | 1-2 | AAAGRRAGCTCATGGCCC start pos = 438 approx Tm = 60.16 | |
| | | approx % gc = 55.56 length = 18 | |
| | 1-2 | rev comp = GGGCCATGAGCTYYCTTT | 70 |
| 41 | 1-2 | YRACCGGAGTATCAGCATCATG start pos = 466 approx | |
| | | Tm = 59.98 approx % gc = 45.45 length = 22 | |
| | 1-2 | rev comp = CATGATGCTGATACTCCGGTYR | 71 |
| 42 | 1-2 | GCATCATGCTCCCATAAYG start pos = 480 approx Tm = 60.05 | |
| | | approx % gc = 52.63 length = 19 | |
| | 1-2 | rev comp = CRTTATGGGAGCATGATGC | 72 |
| 43 | 1-2 | TGCTCCCATAAYGGGRAA start pos = 486 approx Tm = 60.00 | |
| | | approx % gc = 50.00 length = 18 | |
| | 1-2 | rev comp = TTYCCCRTTATGGGAGCA | 73 |
| 44 | 1-2 | AGTTTYTACARAAATTTGCTATGGCT start pos = 507 approx | |
| | | Tm = 59.89 approx % gc = 26.92 length = 26 | |
| | 1-2 | rev comp = AGCCATAGCAAATTTYTGTARAAACT | 74 |
| 45 | 1-2 | AAATTTGCTATGGCTGACGR start pos = 518 approx Tm = 60.10 | |
| | | approx % gc = 45.00 length = 20 | |
| | 1-2 | rev comp = YCGTCAGCCATAGCAAATTT | 75 |
| 46 | 1-2 | TGGCTGACGRGGAARAATG start pos = 528 approx Tm = 59.93 | |
| | | approx % gc = 52.63 length = 19 | |
| | 1-2 | rev comp = CATTYTTCCYCGTCAGCCA | 76 |
| 47 | 1-2 | GTTTGTAYCCAAACCTGAGCA start pos = 547 approx Tm = 60.02 | |
| | | approx % gc = 47.62 length = 21 | |
| | 1-2 | rev comp = TGCTCAGGTTTGGRTACAAAC | 77 |
| 48 | 1-2 | TATGYAAACAACAAAGARAAAGAAGT start pos = 573 approx | |
| | | Tm = 59.79 approx % gc = 30.77 length = 26 | |
| | 1-2 | rev comp = ACTTCTTTYTCTTTGTTGTTTRCATA | 78 |
| 49 | 1-2 | ARAAAGAAGTCCTTGTRCTATGGGG start pos = 589 approx | |
| | | Tm = 60.28 approx % gc = 44.00 length = 25 | |
| | 1-2 | rev comp = CCCCATAGYACAAGGACTTCTTTYT | 79 |
| 50 | 1-2 | GTCCTTGTRCTATGGGGTGTTCA start pos = 597 approx | |
| | | Tm = 60.16 approx % gc = 47.83 length = 23 | |
| | 1-2 | rev comp = TGAACACCCCATAGYACAAGGAC | 80 |
| 51 | 1-2 | GTTCATCACCCRCCTAACAT start pos = 615 approx Tm = 59.82 | |
| | | approx % gc = 50.00 length = 20 | |
| | 1-2 | rev comp = ATGTTAGGYGGGTGATGAAC | 81 |
| 52 | 1-2 | GCYCTCTAYCATACAGAAAATGCT start pos = 648 approx | |
| | | Tm = 60.02 approx % gc = 41.67 length = 24 | |
| | 1-2 | rev comp = AGCATTTTCTGTATGRTAGAGRGC | 82 |
| 53 | 1-2 | TATAGCAGRARATTCACCCCAGA start pos = 696 approx | |
| | | Tm = 60.10 approx % gc = 43.48 length = 23 | |
| | 1-2 | rev comp = TCTGGGGTGAATYTYCTGCTATA | 83 |
| 54 | 1-2 | AAATAGCCAAAAGACCCAARGTRAG start pos = 718 approx | |
| | | Tm = 60.27 approx % gc = 36.00 length = 25 | |
| | 1-2 | rev comp = CTYACYTTGGGTCTTTTGGCTATTT | 84 |
| 55 | 1-2 | TRAGAGATCARGAAGGAAGAATCAA start pos = 739 approx | |
| | | Tm = 59.76 approx % gc = 36.00 length = 25 | |
| | 1-2 | rev comp = TTGATTCTTCCTTCYTGATCTCTYA | 85 |
| 56 | 1-2 | TKGAACCCGGGGAYACAAT start pos = 781 approx Tm = 60.00 | |
| | | approx % gc = 47.37 length = 19 | |
| | 1-2 | rev comp = ATTGTRTCCCCGGGTTCMA | 86 |
| 57 | 1-2 | GGGAYACAATAATATTTGAGGCAAAT start pos = 790 approx | |
| | | Tm = 59.78 approx % gc = 30.77 length = 26 | |
| | 1-2 | rev comp = ATTTGCCTCAAATATTATTGTRTCCC | 87 |
| 58 | 1-2 | CAAATGGAAATCTAATAGCRCCAWG start pos = 811 approx | |
| | | Tm = 60.25 approx % gc = 36.00 length = 25 | |
| | 1-2 | rev comp = CWTGGYGCTATTAGATTTCCATTTG | 88 |
| 59 | 1-2 | ATCAGGAATCAKCAMCTCAAATG start pos = 866 approx | |
| | | Tm = 60.20 approx % gc = 39.13 length = 23 | |
| | 1-2 | rev comp = CATTTGAGKTGMTGATTCCTGAT | 89 |
| 60 | 1-2 | TGCACCAATGGRTGAATG start pos = 887 approx Tm = 59.88 | |
| | | approx % gc = 50.00 length = 18 | |
| | 1-2 | rev comp = CATTCAYCCATTGGTGCA | 90 |

In some embodiments, the primer has a DNA sequence that corresponds to the RNA sequence of a well conserved region of the HA gene of influenza A virus subtype H3 as set out in Table 2. Such primers may be used as a forward or reverse primer when sequencing or amplifying a first strand DNA reversed transcribed from the HA gene.

TABLE 2

Forward and reverse primers for the H3 Gene. Bold primers
indicate a primers suitable for amplification of the whole
peptide region. ID F denotes forward primer and ID R
reverse primer for the complementary sequence.
Additional primers can be found at FIGS. 17-19.

| | | | |
|---|---|---|---|
| ID F | | | ID R |
| No: | Deg | H3 Primer | No: |

| | | | |
|---|---|---|---|
| 91 | 0 | TCTATTGGGAGACCCTCAGTGT start pos = 263 approx | |
| | | Tm = 59.99 approx % gc = 50.00 | |
| 92 | 0 | TATTGGGAGACCCTCAGTGTG start pos = 265 approx Tm = 59.97 | |
| | | approx % gc = 52.38 | |
| 93 | 0 | ATTGGGAGACCCTCAGTGTG start pos = 266 approx Tm = 59.96 | |
| | | approx % gc = 55.00 | |
| 94 | 0 | TTGGGAGACCCTCAGTGTG start pos = 267 approx Tm = 59.64 | |
| | | approx % gc = 57.89 | |
| 95 | 0 | GACCCTCAGTGTGATGGCTT start pos = 273 approx Tm = 60.12 | |
| | | approx % gc = 55.00 | |
| 96 | 0 | CAGTGTGATGGCTTCCAAAAT start pos = 279 approx Tm = 59.99 | |
| | | approx % gc = 42.86 | |
| 97 | 0 | CAGTGTGATGGCTTCCAAAATA start pos = 279 approx | |
| | | Tm = 60.00 approx % gc = 40.91 | |
| 98 | 0 | CTTCCAAAATAAGAAATGGGACC start pos = 290 approx | |
| | | Tm = 60.06 approx % gc = 39.13 | |
| 99 | 0 | ACCTTTTTGTTGAACGCAGC start pos = 310 approx Tm = 60.29 | |
| | | approx % gc = 45.00 | |
| 100 | 0 | TGTTGAACGCAGCAAAGC start pos = 317 approx Tm = 59.70 | |
| | | approx % gc = 50.00 | |
| 101 | 0 | TGAACGCAGCAAAGCCTAC start pos = 320 approx Tm = 60.15 | |
| | | approx % gc = 52.63 | |
| 102 | 0 | TCCGGCACACTGGAGTTT start pos = 399 approx Tm = 60.25 | |
| | | approx % gc = 55.56 length = 18 | |
| | | rev comp = AAACTCCAGTGTGCCGGA | 151 |
| 103 | 0 | CGGCACACTGGAGTTTAACA start pos = 401 approx Tm = 59.76 | |
| | | approx % gc = 50.00 length = 20 | |
| | | rev comp = TGTTAAACTCCAGTGTGCCG | 152 |
| 104 | 0 | AATTGGACTGGAGTCACTCAAAA start pos = 432 approx | |
| | | Tm = 60.03 approx % gc = 39.13 length = 23 | |
| | | rev comp = TTTTGAGTGACTCCAGTCCAATT | 153 |
| 105 | 0 | TGGAACAAGCTCTGCTTGC start pos = 455 approx Tm = 60.28 | |
| | | approx % gc = 52.63 length = 19 | |
| | | rev comp = GCAAGCAGAGCTTGTTCCA | 154 |
| 106 | 0 | CTTTAGTAGATTGAATTGGTTGACCC start pos = 497 approx | |
| | | Tm = 60.47 approx % gc = 38.46 length = 26 | |
| | | rev comp = GGGTCAACCAATTCAATCTACTAAAG | 155 |
| 107 | 0 | GATTGAATTGGTTGACCCACTT start pos = 505 approx | |
| | | Tm = 60.10 approx % gc = 40.91 length = 22 | |
| | | rev comp = AAGTGGGTCAACCAATTCAATC | 156 |
| 108 | 0 | TATGCTCAAGCATCAGGAAGAA start pos = 639 approx | |
| | | Tm = 59.98 approx % gc = 40.91 length = 22 | |
| | | rev comp = TTCTTCCTGATGCTTGAGCATA | 157 |
| 109 | 0 | ATGCTCAAGCATCAGGAAGAA start pos = 640 approx Tm = 59.97 | |
| | | approx % gc = 42.86 length = 21 | |
| | | rev comp = TTCTTCCTGATGCTTGAGCAT | 158 |
| 110 | 0 | CAAGCATCAGGAAGAATCACAG start pos = 645 approx | |
| | | Tm = 59.88 approx % gc = 45.45 length = 22 | |
| | | rev comp = CTGTGATTCTTCCTGATGCTTG | 159 |
| 111 | 0 | GGAAGAATCACAGTCTCTACCAAAA start pos = 654 approx | |
| | | Tm = 60.05 approx % gc = 40.00 length = 25 | |
| | | rev comp = TTTTGGTAGAGACTGTGATTCTTCC | 160 |
| 112 | 0 | GGACAATAGTAAAACCGGGAGAC start pos = 757 approx | |
| | | Tm = 60.12 approx % gc = 47.83 length = 23 | |
| | | rev comp = GTCTCCCGGTTTTACTATTGTCC | 161 |
| 113 | 0 | GACAATAGTAAAACCGGGAGACATAC start pos = 758 approx | |
| | | Tm = 60.39 approx % gc = 42.31 length = 26 | |
| | | rev comp = GTATGTCTCCCGGTTTTACTATTGTC | 162 |
| 114 | 0 | GTAAAACCGGGAGACATACTTTTG start pos = 765 approx | |
| | | Tm = 60.16 approx % gc = 41.67 length = 24 | |
| | | rev comp = CAAAAGTATGTCTCCCGGTTTTAC | 163 |
| 115 | 0 | AAACCGGGAGACATACTTTTGA start pos = 768 approx | |
| | | Tm = 59.87 approx % gc = 40.91 length = 22 | |
| | | rev comp = TCAAAAGTATGTCTCCCGGTTT | 164 |
| 116 | 0 | AACCGGGAGACATACTTTTGATTA start pos = 769 approx | |
| | | Tm = 60.13 approx % gc = 37.50 length = 24 | |
| | | rev comp = TAATCAAAAGTATGTCTCCCGGTT | 165 |
| 117 | 0 | GACATACTTTTGATTAACAGCACAGG start pos = 777 approx | |
| | | Tm = 60.33 approx % gc = 38.46 length = 26 | |
| | | rev comp = CCTGTGCTGTTAATCAAAAGTATGTC | 166 |
| 118 | 0 | TACTTTTGATTAACAGCACAGGGA start pos = 781 approx | |
| | | Tm = 60.06 approx % gc = 37.50 length = 24 | |
| | | rev comp = TCCCTGTGCTGTTAATCAAAAGTA | 167 |
| 119 | 0 | TGATTAACAGCACAGGGAATCTAA start pos = 787 approx | |
| | | Tm = 60.02 approx % gc = 37.50 length = 24 | |
| | | rev comp = TTAGATTCCCTGTGCTGTTAATCA | 168 |
| 120 | 0 | GGGTTACTTCAAAATACGAAGTGG start pos = 821 approx | |
| | | Tm = 60.16 approx % gc = 41.67 length = 24 | |
| | | rev comp = CCACTTCGTATTTTGAAGTAACCC | 169 |
| 121 | 0 | CAAAATACGAAGTGGGAAAAGC start pos = 830 approx | |
| | | Tm = 60.00 approx % gc = 40.91 length = 22 | |
| | | rev comp = GCTTTTCCCACTTCGTATTTTG | 170 |
| 122 | 0 | AAAGCTCAATAATGAGATCAGATGC start pos = 847 approx | |
| | | Tm = 60.13 approx % gc = 36.00 length = 25 | |
| | | rev comp = GCATCTGATCTCATTATTGAGCTTT | 171 |
| 123 | 1-2 | TGAAGTTACTAATGCTACTGARCTGG start pos = 158 approx | |
| | | Tm = 60.36 approx % gc = 42.31 | |
| 124 | 1-2 | TGARCTGGTTCAGAGTTCCTCA start pos = 176 approx | |
| | | Tm = 59.88 approx % gc = 45.45 | |
| 125 | 1-2 | CAGAGTTCCTCAACAGGTGRAATAT start pos = 186 approx | |
| | | Tm = 59.95 approx % gc = 40.00 | |
| 126 | 1-2 | AACAGGTGRAATATGCGACAG start pos = 197 approx Tm = 60.01 | |
| | | approx % gc = 47.62 | |
| 127 | 1-2 | AGYCCTCATCAGATCCTTGAT start pos = 216 approx Tm = 60.05 | |
| | | approx % gc = 47.62 | |
| 128 | 1-2 | AAGCCTACAGCAACTGTTAYCCTTAT start pos = 331 approx | |
| | | Tm = 60.01 approx % gc = 38.46 length = 26 | |
| | | rev comp = ATAAGGRTAACAGTTGCTGTAGGCTT | 172 |
| 129 | 1-2 | CAGCAACTGTTAYCCTTATGATGTG start pos = 338 approx | |
| | | Tm = 59.97 approx % gc = 40.00 length = 25 | |
| | | rev comp = CACATCATAAGGRTAACAGTTGCTG | 173 |
| 130 | 1-2 | AYCCTTATGATGTGCCGG start pos = 349 approx Tm = 59.32 | |
| | | approx % gc = 55.56 length = 18 | |
| | | rev comp = CCGGCACATCATAAGGRT | 174 |
| 131 | 1-2 | ATGTGCCGGATTATGCCTY start pos = 358 approx Tm = 60.31 | |
| | | approx % gc = 47.37 length = 19 | |
| | | rev comp = RAGGCATAATCCGGCACAT | 175 |
| 132 | 1-2 | GATTATGCCTYCCTTAGGTCACT start pos = 366 approx | |
| | | Tm = 59.88 approx % gc = 47.83 length = 23 | |
| | | rev comp = AGTGACCTAAGGRAGGCATAATC | 176 |
| 133 | 1-2 | CTYCCTTAGGTCACTARTTGCCT start pos = 374 approx | |
| | | Tm = 60.03 approx % gc = 47.83 length = 23 | |
| | | rev comp = AGGCAAYTAGTGACCTAAGGRAG | 177 |
| 134 | 1-2 | ACTARTTGCCTCATCCGGC start pos = 386 approx Tm = 60.05 | |
| | | approx % gc = 52.63 length = 19 | |
| | | rev comp = GCCGGATGAGGCAAYTAGT | 178 |
| 135 | 1-2 | CACTGGAGTTTAACAATGARAGCTT start pos = 406 approx | |
| | | Tm = 60.10 approx % gc = 36.00 length = 25 | |
| | | rev comp = AAGCTYTCATTGTTAAACTCCAGTG | 179 |
| 136 | 1-2 | GGTTGACCCACTTAAAATTCAAATAY start pos = 514 approx | |
| | | Tm = 60.25 approx % gc = 34.62 length = 26 | |
| | | rev comp = RTATTTGAATTTTAAGTGGGTCAACC | 180 |
| 136 | 1-2 | CTTAAAATTCAAATAYCCAGCATTG start pos = 524 approx | |
| | | Tm = 60.13 approx % gc = 32.00 length = 25 | |
| | | rev comp = CAATGCTGGRTATTTGAATTTTAAG | 181 |
| 137 | 1-2 | CAAATAYCCAGCATTGAAYGT start pos = 533 approx Tm = 59.87 | |
| | | approx % gc = 42.86 length = 21 | |
| | | rev comp = ACRTTCAATGCTGGRTATTTG | 182 |
| 138 | 1-2 | YCCAGCATTGAAYGTGACTAT start pos = 539 approx Tm = 60.01 | |
| | | approx % gc = 47.62 length = 21 | |
| | | rev comp = ATAGTCACRTTCAATGCTGGR | 183 |
| 139 | 1-2 | GACTATGCCAAACAATGAARAATTT start pos = 554 approx | |
| | | Tm = 59.81 approx % gc = 32.00 length = 25 | |
| | | rev comp = AAATTYTTCATTGTTTGGCATAGTC | 184 |
| 140 | 1-2 | GGGTTCACCACCCRGGTA start pos = 598 approx Tm = 59.15 | |
| | | approx % gc = 61.11 length = 18 | |
| | | rev comp = TACCYGGGTGGTGAACCC | 185 |
| 141 | 1-2 | CTRTATGCTCAAGCATCAGGAAGA start pos = 636 approx | |
| | | Tm = 59.90 approx % gc = 41.67 length = 24 | |
| | | rev comp = TCTTCCTGATGCTTGAGCATAYAG | 186 |
| 142 | 1-2 | TCACAGTCTCTACCAAAAGRAGC start pos = 661 approx | |
| | | Tm = 59.94 approx % gc = 47.83 length = 23 | |
| | | rev comp = GCTYCTTTTGGTAGAGACTGTGA | 187 |
| 143 | 1-2 | CTACCAAAAGRAGCCAACAAAC start pos = 670 approx | |
| | | Tm = 60.04 approx % gc = 45.45 length = 22 | |
| | | rev comp = GTTTGTTGGCTYCTTTTGGTAG | 188 |
| 144 | 1-2 | GRAGCCAACAAACTGTAATCCC start pos = 679 approx | |
| | | Tm = 59.88 approx % gc = 45.45 length = 22 | |
| | | rev comp = GGGATTACAGTTTGTTGGCTYC | 189 |
| 145 | 1-2 | ACTGTAATCCCGAATATCGGR start pos = 690 approx Tm = 60.05 | |
| | | approx % gc = 47.62 length = 21 | |
| | | rev comp = YCCGATATTCGGGATTACAGT | 190 |
| 146 | 1-2 | TCCCCAGYAGAATAAGCATM start pos = 733 approx Tm = 60.18 | |
| | | approx % gc = 50.00 length = 20 | |
| | | rev comp = KATGCTTATTCTRCTGGGGA | 191 |
| 147 | 1-2 | GYAGAATAAGCATMTATTGGACAATA start pos = 739 approx | |
| | | Tm = 59.93 approx % gc = 34.62 length = 26 | |
| | | rev comp = TATTGTCCAATAKATGCTTATTCTRC | 192 |
| 148 | 1-2 | ATCTAATTGCTCCTMGGGGT start pos = 805 approx Tm = 59.92 | |
| | | approx % gc = 50.00 length = 20 | |
| | | rev comp = ACCCCKAGGAGCAATTAGAT | 193 |
| 149 | 1-2 | GCTCCTMGGGGTTACTTCA start pos = 813 approx Tm = 60.20 | |
| | | approx % gc = 57.89 length = 19 | |
| | | rev comp = TGAAGTAACCCCKAGGAGC | |
| 150 | | AAACCATTTCAAAATGTAAAYAGGA start pos = 930 approx | |
| | | Tm = 60.03 approx % gc = 28.00 length = 25 | |
| | 1-2 | rev comp = TCCTRTTTACATTTTGAAATGGTTT | 194 |

TABLE 3

Exemplary forward and reverse primers for the H5 Gene.
Bold primers indicate a primers suitable for amplification of
the whole peptide region. ID F denotes forward primer and ID
R reverse primer for the complementary sequence.
Additional primers can be found at FIGS. 17-19.

| ID F | | | ID R |
|---|---|---|---|
| No: | Deg | H5 Primer | No: |
| 195 | 0 | CTGTTACACATGCCCAAGACATA start pos = 150 approx Tm = 59.93 approx % gc = 43.48 | |
| | | length = 23 | |
| 196 | 0 | GTGTAGCTGGATGGCTCCTC start pos = 243 approx Tm = 59.83 approx % gc = 60.00 | |
| | | length = 20 | |
| 197 | 0 | TAGCTGGATGGCTCCTCG start pos = 246 approx Tm = 60.05 approx % gc = 61.11 | |
| | | length = 18 | |
| 198 | | | |
| 199 | 0 | CGGAATGGTCTTACATAGTGGAG start pos = 297 approx Tm = 59.90 approx % gc = 47.83 | |
| | | length = 23 | |
| | 0 | rev comp = CTCCACTATGTAAGACCATTCCG | 258 |
| 200 | 0 | ATGGTCTTACATAGTGGAGAAGGC start pos = 301 approx Tm = 59.93 approx % gc = 45.83 | |
| | | length = 24 | |
| | 0 | rev comp = GCCTTCTCCACTATGTAAGACCAT | 259 |
| 201 | 0 | TCTTACATAGTGGAGAAGGCCAA start pos = 305 approx Tm = 60.14 approx % gc = 43.48 | |
| | | length = 23 | |
| | 0 | rev comp = TTGGCCTTCTCCACTATGTAAGA | 260 |
| 202 | 0 | ATTGAGCAGAATAAACCATTTTGAG start pos = 388 approx Tm = 59.92 approx | |
| | | % gc = 32.00 length = 25 | |
| | 0 | rev comp = CTCAAAATGGTTTATTCTGCTCAAT | 261 |
| 203 | 0 | AGCAGAATAAACCATTTTGAGAAAAT start pos = 392 approx Tm = 59.84 approx | |
| | | % gc = 26.92 length = 26 | |
| | 0 | rev comp = ATTTTCTCAAAATGGTTTATTCTGCT | 262 |
| 204 | 0 | TGTGGTATGGCTTATCAAAAAGAA start pos = 514 approx Tm = 59.90 approx % gc = 33.333 | |
| | | length = 24 | |
| | 0 | rev comp = TTCTTTTTGATAAGCCATACCACA | 263 |
| 205 | 0 | GATCCAAAGTAAACGGGCAA start pos = 723 approx Tm = 59.94 approx % gc = 45.00 | |
| | | length = 20 | |
| | 0 | rev comp = TTGCCCGTTTACTTTGGATC | 264 |
| 206 | 0 | TGCTCCAGAATATGCATACAAAT start pos = 820 approx Tm = 59.90 approx % gc = 33.33 | |
| | | length = 24 | |
| | 0 | rev comp = ATTTTGTATGCATATTCTGGAGCA | 265 |
| 207 | 0 | CCAGAATATGCATACAAAATTGTCA start pos = 824 approx Tm = 60.14 approx | |
| | | % gc = 32.00 length = 25 | |
| | 0 | rev comp = TGACAATTTTGTATGCATATTCTGG | 266 |
| 208 | 0 | ATACAAAATTGTCAAGAAAGGGGA start pos = 835 approx Tm = 60.11 approx % gc = 33.33 | |
| | | length = 24 | |
| | 0 | rev comp = TCCCCTTTCTTGACAATTTTGTAT | 267 |
| 209 | 0 | AATTGTCAAGAAAGGGGACTCA start pos = 841 approx Tm = 59.98 approx % gc = 40.91 | |
| | | length = 22 | |
| | 0 | rev comp = TGAGTCCCCTTTCTTGACAATT | 268 |
| 210 | 0 | TATGGTAACTGCAACACCAAGTC start pos = 887 approx Tm = 59.97 approx % gc = 43.48 | |
| | | length = 23 | |
| | 0 | rev comp = CACTTGGTGTTGCAGTTACCATA | 269 |
| 211 | 0 | ATGGTAACTGCAACACCAAGTG start pos = 888 approx Tm = 59.96 approx % gc = 45.45 | |
| | | length = 22 | |
| | 0 | rev comp = CACTTGGTGTTGCAGTTACCAT | 270 |
| 212 | 0 | ATACACCCTCTCACCATCGG start pos = 956 approx Tm = 59.80 approx % gc = 55.00 | |
| | | length = 20 | |
| | 0 | rev comp = CCGATGGTGAGAGGGTGTAT | 271 |
| 213 | 0 | ACACCCTCTCACCATCGG start pos = 958 approx Tm = 59.45 approx % gc = 61.11 | |
| | | length = 18 | |
| | 0 | rev comp = CCGATGGTGAGAGGGTGT | 272 |
| 214 | 0 | GAATGCCCCAAATATGTGAAAT start pos = 977 approx Tm = 59.92 approx % gc = 36.36 | |
| | | length = 22 | |
| | 0 | rev comp = ATTTCACATATTTGGGGCATTC | 273 |
| 215 | 1-2 | TRAGAGATTGTAGTGTAGCTGGATGG start pos = 231 approx Tm = 60.10 approx | |
| | | % gc = 42.31 | |
| 216 | 1-2 | RAGAGATTGTAGTGTAGCTGGATGG start pos = 232 approx Tm = 60.09 approx | |
| | | % gc = 44.00 | |
| 217 | 1-2 | CCTCGGRAACCCRATGTG start pos = 259 approx Tm = 59.89 approx % gc = 55.56 | |
| 218 | 1-2 | CTCGGRAACCCRATGTGTG start pos = 260 approx Tm = 59.94 approx % gc = 52.63 | |
| 219 | 1-2 | AACCCRATGTGTGACGAATT start pos = 266 approx Tm = 60.24 approx % gc = 45.00 | |
| 220 | 1-2 | AATTCATCAATGTRCCGGA start pos = 282 approx Tm = 59.87 approx % gc = 42.11 | |
| 221 | 1-2 | ATTCATCAATGTRCCGGAAT start pos = 283 approx Tm = 60.16 approx % gc = 40.00 | |
| 222 | 1-2 | TTCATCAATGTRCCGGAAT start pos = 284 approx Tm = 59.87 approx % gc = 42.11 | |
| 223 | 1-2 | TCATCAATGTRCCGGAATGGT start pos = 285 approx Tm = 60.07 approx % gc = 42.86 | |
| 224 | 1-2 | GTRCCGGAATGGTCTTACATA start pos = 293 approx Tm = 59.84 approx % gc = 47.62 | |
| 225 | 1-2 | TAGTGGAGAAGGCCAAYCC start pos = 312 approx Tm = 60.06 approx % gc = 57.89 | |
| | | length = 19 | |
| | 1-2 | rev comp = GGRTTGGCCTTCTCCACTA | 274 |
| 226 | 1-2 | YCAATGACCTCTGTTWCCCAG start pos = 333 approx Tm = 60.10 approx % gc = 47.62 | |
| | | length = 21 | |
| | 1-2 | rev comp = CTGGGWAACAGAGGTCATTGR | 275 |
| 227 | 1-2 | TTTCAAYGACTATGAAGAAYTGAAAC start pos = 358 approx Tm = 60.10 approx | |
| | | % gc = 34.62 length = 26 | |
| | 1-2 | rev comp = GTTTCARTTCTTCATAGTCRTTGAAA | 276 |
| 228 | 1-2 | YTGAAACAYCTATTGAGCAGAATAAA start pos = 377 approx Tm = 60.08 approx | |
| | | % gc = 34.62 length = 26 | |
| | 1-2 | rev comp = TTTATTCTGCTCAATAGRTGTTTCAR | 277 |
| 229 | 1-2 | AACCATTTTGAGAAAATTCARATCA start pos = 401 approx Tm = 60.10 approx | |
| | | % gc = 24.00 length = 25 | |
| | 1-2 | rev comp = TGATYTGAATTTTCTCAAAATGGTT | 278 |
| 230 | 1-2 | GAAAATTCARATCATCCCCAAA start pos = 412 approx Tm = 60.00 approx % gc = 31.82 | |
| | | length = 22 | |
| | 1-2 | rev comp = TTTGGGGATGATYTGAATTTTC | 279 |
| 231 | 1-2 | TCARATCATCCCCAAAARTTCT start pos = 418 approx Tm = 59.94 approx % gc = 40.91 | |
| | | length = 22 | |
| | 1-2 | rev comp = AGAAYTTTTGGGGATGATYTGA | 280 |
| 232 | 1-2 | CCCAAAARTTCTTGGTCCR start pos = 428 approx Tm = 60.47 approx % gc = 52.63 | |
| | | length = 19 | |
| | 1-2 | rev comp = YGGACCAAGAAYTTTTGGG | 281 |
| 233 | 1-2 | TTTTYAGRAATGTGGTATGGC start pos = 504 approx Tm = 59.81 approx % gc = 42.86 | |
| | | length = 21 | |
| | 1-2 | rev comp = GCCATACCACATTYCTRAAAA | 282 |
| 234 | 1-2 | TYAGRAATGTGGTATGGCTTATCAAA start pos = 507 approx Tm = 60.14 approx | |
| | | % gc = 30.77 length = 26 | |
| | 1-2 | rev comp = TTTGATAAGCCATACCACATTYCTRA | 283 |
| 235 | 1-2 | CATACCCAACAATAAAGARRAGCTA start pos = 543 approx Tm = 59.94 approx | |
| | | % gc = 40.00 length = 25 | |
| | 1-2 | rev comp = TAGCTYYTCTTTATTGTTGGGTATG | 284 |
| 236 | 1-2 | GCTACAATAATACCAACCARGAAGA start pos = 564 approx Tm = 59.83 approx | |
| | | % gc = 40.00 length = 25 | |
| | 1-2 | rev comp = TCTTCYTGGTTGGTATTATTGTAGC | 285 |
| 237 | 1-2 | TACCAACCARGAAGATCTTTTGR start pos = 574 approx Tm = 59.99 approx % gc = 39.13 | |
| | | length = 23 | |
| | 1-2 | rev comp = YCAAAAGATCTTCYTGGTTGGTA | 286 |
| 238 | 1-2 | TYCTAATGATGSGGCAGAG start pos = 616 approx Tm = 59.92 approx % gc = 52.63 | |
| | | length = 19 | |
| | 1-2 | rev comp = CTCTGCCSCATCATTAGRA | 287 |
| 239 | 1-2 | AATGATGSGGCAGAGCAG start pos = 620 approx Tm = 59.74 approx % gc = 55.56 | |
| | | length = 18 | |
| | 1-2 | rev comp = CTGCTCTGCCSCATCATT | 288 |
| 240 | 1-2 | ARGCTMTATCAAAACCCAACCA start pos = 641 approx Tm = 60.00 approx % gc = 40.91 | |
| | | length = 22 | |
| | 1-2 | rev comp = TGGTTGGGTTTTGATAKAGCYT | 289 |
| 241 | 1-2 | CAAAACCCAACCACCTAYATTT start pos = 650 approx Tm = 60.01 approx % gc = 40.91 | |
| | | length = 22 | |
| | 1-2 | rev comp = AAATRTAGGTGGTTGGGTTTTG | 290 |
| 242 | 1-2 | TGGGACMTCAACACTAAACCAG start pos = 676 approx Tm = 59.89 approx % gc = 45.45 | |
| | | length = 22 | |
| | 1-2 | rev comp = CTGGTTTAGTGTTGAKGTCCCA | 291 |
| 243 | 1-2 | AAARTGGAAGGATGGAKTTCTTC start pos = 741 approx Tm = 59.99 approx % gc = 43.48 | |
| | | length = 23 | |
| | 1-2 | rev comp = GAAGAAMTCCATCCTTCCAYTTT | 292 |
| 244 | 1-2 | GGATGGAKTTCTTCTGGRC start pos = 750 approx Tm = 59.60 approx % gc = 57.89 | |
| | | length = 19 | |
| | 1-2 | rev comp = GYCCAGAAGAAMTCCATCC | 293 |
| 245 | 1-2 | GATGGAKTTCTTCTGGRCAATTTTA start pos = 751 approx Tm = 59.90 approx | |
| | | % gc = 36.00 length = 25 | |
| | 1-2 | rev comp = TAAAATTGYCCAGAAGAAMTCCATC | 294 |
| 246 | 1-2 | CTTCTGGRCAATTTTAAAACCKAAT start pos = 760 approx Tm = 60.13 approx | |
| | | % gc = 32.00 length = 25 | |
| | 1-2 | rev comp = ATTMGGTTTTAAAATTGYCCAGAAG | 295 |
| 247 | 1-2 | TAAAACCKAATGATGCAATCAACTTC start pos = 774 approx Tm = 59.83 approx | |
| | | % gc = 32.77 length = 26 | |
| | 1-2 | rev comp = GAAGTTGATTGCATCATTMGGTTTTA | 296 |
| 248 | 1-2 | AAACCKAATGATGCAATCAAC start pos = 776 approx Tm = 59.82 approx % gc = 38.10 | |
| | | length = 21 | |
| | 1-2 | rev comp = GTTGATTGCATCATTMGGTTT | 297 |
| 249 | 1-2 | AAAGGGGACTCARCAATTATGAAA start pos = 851 approx Tm = 60.11 approx % gc = 33.33 | |
| | | length = 24 | |
| | 1-2 | rev comp = TTTCATAATTGYTGAGTCCCCTTT | 298 |
| 250 | 1-2 | ACTCARCAATTATGAAAAGTGAAKTG start pos = 858 approx Tm = 60.11 approx | |
| | | % gc = 34.62 length = 26 | |
| | 1-2 | rev comp = CAMTTCACTTTTCATAATTGYTGAGT | 299 |
| 251 | 1-2 | AAGTGAAKTGGAATATGGTAACTGC start pos = 874 approx Tm = 59.86 approx | |
| | | % gc = 40.00 length = 25 | |
| | 1-2 | rev comp = GCAGTTACCATATTCCAMTTCACTT | 300 |
| 252 | 1-2 | TGTCAAACTCCAATRGGGGC start pos = 908 approx Tm = 59.93 approx % gc = 50.00 | |
| | | length = 20 | |
| | 1-2 | rev comp = GCCCCYATTGGAGTTTGACA | 301 |
| 253 | 1-2 | AAACTCCAATRGGGGCGATAA start pos = 912 approx Tm = 59.82 approx % gc = 42.86 | |
| | | length = 21 | |
| | 1-2 | rev comp = TTATCGCCCCYATTGGAGTTT | 302 |
| 254 | 1-2 | CAATRGGGGCGATAAACTC start pos = 918 approx Tm = 60.28 approx % gc = 52.63 | |
| | | length = 19 | |
| | 1-2 | rev comp = GAGTTTATCGCCCCYATTG | 303 |
| 255 | 1-2 | TAAACTCTAGTATGCCATTCCACAAY start pos = 930 approx Tm = 59.87 approx | |
| | | % gc = 38.46 length = 26 | |
| | 1-2 | rev comp = RTTGTGGAATGGCATACTAGAGTTTA | 304 |
| 256 | 1-2 | TAGTATGCCATTCCACAAYATACAC start pos = 937 approx Tm = 60.08 approx | |
| | | % gc = 40.00 length = 25 | |
| | 1-2 | rev comp = GTGTATRTTGTGGAATGGCCATACTA | 305 |
| 257 | 1-2 | TCCACAAYATACACCCTCTCACC start pos = 948 approx Tm = 60.12 approx % gc = 47.83 | |
| | | length = 23 | |
| | 1-2 | rev comp = GGTGAGAGGGTGTATRTTGTGGA | 306 |

Furthermore, a skilled person will understand that, although the primers are based on conserved sequences, one or more bases within the conserved sequences can be substituted, inserted or deleted, provided that the mutated primer will still hybridize with the target sequence in a sample with the same or similar stringency as the original primer sequence. Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 50 C lower than the thermal melting point for the specific sequence at a defined ionic strength and pH.

A skilled person will understand that having multiple substitution mutations in a short sequence will decrease the strength of hybridization of the primer to the complement of the original, unmutated primer, and that the spacing and location of the mutations within the primer sequence will also affect the strength or stringency of hybridization. Furthermore, a skilled person will understand that insertion or deletion of one or more nucleotides in a short sequence will also decrease the strength of hybridization of the primer to the complement of the original, unmutated primer, and that having insertions or deletions of one or more nucleotides in more than one location in a short sequence may significantly alter the hybridization of the primer to the complement of the unmutated sequence.

In some embodiments, the primer may be modified with a label to allow for detection of the primer or a DNA product synthesized or extended from the primer. For example, the label may be a fluorescent label, a chemiluminescent label, a coloured dye label, a radioactive label, a radiopaque label, a protein including an enzyme, a peptide or a ligand for example biotin.

Alternatively, the additional sequence may not be directed to the HA gene, but may be a sequence, for example, that is recognised by a protein or an enzyme, for example a restriction enzyme, or that is complementary to a nucleic acid sequence that is used for detection, for example, that is complementary to a probe that may be labelled. A skilled person will understand that there will be an optimum length and sequence for the primer, depending on the application for which the primer is to be used, so as to suitably limit the number and type of any such additional sequences. For example, a PCR primer should not be of such length or sequence that the temperature above which it no longer specifically binds to the template approaches the temperature at which the extension by polymerase occurs.

Skilled artisan also understands that primers can surround at least one peptide epitope of the present invention, e.g. peptide 1 region (prepeptide 1, peptide1 and/or postpeptide1), or at least two regions, e.g. peptide 1 and peptide 2 and their surrounding regions. Alternatively, two peptide regions can encompass peptides 2 and 4, or 4 and 3. In the other words, primers can be in between peptide sequences. Furthermore, primers can encompass at least three peptide regions, e.g. peptide 1, 2 and 4, or 2, 4 and 3. One embodiment favors primers which bind upstream of peptide 1 and downstream of peptide 3, i.e. encompassing the whole large binding region. This region is about 500-520 nucleotides and resulting fragment can be about 500, 510, 520, 530, 540, 550, 560 or 570 by of length. Alternatively, is some applications about 600, 700, 800 or longer by fragments are desired.

Skilled artisan also understands that a primer sequence may be located in between peptide epitopes or motifs.

Skilled artisan further understands that in some applications it is preferred to use primers which bind to nucleotides corresponding peptide regions or motifs of the present invention. For example, ID F NOS: 33 and 34 bind to peptide 1 of H1.

Peptide region is defined as a amino acid sequence which encompasses conserved tri- or oligopeptide motifs described herein. For example, conserved peptide motif of peptide 3 of H1 is KVR. Peptide region of KVR means amino acid sequences upstream (toward amino terminus) of KVR and downstream (toward COOH terminus) of KVR, usually 1, 2, 3, 4 or 5 amino acids in length. The upstream and downstream amino acid sequences can also be 6, 7, 8, 9, 10 or longer. These pre and post peptide sequences can be conserved and the invention is directed to identify these conserved sequences. The pre and post peptide sequences can also contain non conserved amino acids which are depicted as X and can be any amino acid or limited to few amino acids which are seen to vary in or between HA gene.

Peptide region also contemplates corresponding nucleotide sequences encoding the amino acids in the region or epitope. Due to degeneracy many nucleotide sequences can encode a single amino acid and are also included in the present invention.

The present invention is directed to all influenza virus A regardless of host species. Host species can be avian, swine, or mammalian. Preferred avian host consist of chicken, duck, and quail. Preferred feline species consist of cat, tiger and leopard. Other preferred mammalians are dog, equine, mouse, seal, whale and mink. Most preferred mammalian is human. Most preferred host is human. Other species include camel. Skilled artisan understands that all influenza A types which infect host species other than human may potentially mutate and infect humans. Therefore the present invention is suitable for screening and anticipating peptide antibodies which are to be administered to humans to treat influenza, alleviate influenza symptoms, to treat and/or alleviate symptoms caused by influenza conditions, for example, secondary infection caused by bacteria. Most preferred is the prevention of influenza symptoms by determining peptide epitopes of an influenza type and administering peptides of the present invention. The determination can be accomplished by using primers of any sequence set forth in ID F/R NOS: 1-306.

Skilled artisan understands to screen for other peptide epitope encompassing and encoding primers using methodology described herein. HA subtypes for additional H1, H3 and H5 can be screened using methodology. Preferably, other HA subtypes like H2, H6-17 can be screened to anticipate their potential threat to mutate and acquire human-to-human or animal-to-human transmission.

The invention is well suited for preventing influenza in a patient. HA subtype is determined using primers of the present invention and peptide epitopes of the present invention are administered into a patient, and immune defense is raised against peptides, thus, against influenza virus.

Of particular preference are the primers set forth in ID F NO: 12 (start at 270) and ID R NO: 89 (end at 866); ID F NO: 95 (start at 273) and ID R NO: 170 (end at 847); and ID F NO: 219 (start at 266) and ID F NOS: 208 and 209 (end at 835 and 841). They encompass the whole peptide binding region, or large binding region. Corresponding nucleotide fragment lengths are about 600-620 by for H1 and H3, respectively.

Therefore, in certain embodiments, the primer consists essentially of the sequence of any one of primers set forth in Tables 1-3 and FIGS. 17-19, meaning the primer may include one or more additional nucleotides, 5′ to, 3′ to, or flanking on either side, of the sequence of any one of primers set forth in Tables 1-3, but that the additional nucleotides should not significantly affect the hybridization of the sequence of any one of primers set forth in Tables 1-3 to a nucleic acid molecule containing the complementary sequence. For example, the addition of several nucleotides on either side of a short primer sequence should not alter the hybridization stringency of the short primer sequence to its complementary sequence even when contained within a larger sequence, to such an extent that the short primer sequence cannot hybridize with the same or similar stringency as when the additional nucleotides are not present. That is, since the regions in the influenza HA gene surrounding the sequences described herein may vary among isolates, a primer consisting essentially of the sequence of any one of primers set forth in Tables 1-3 should not include so much of the viral sequences flanking the conserved sequences described herein so as to affect the sensitivity and ability to detect a wide range of H1, H3 or H5, or preferably H1N1, H3N2 or H5N1 isolates. In certain other embodiments the primer consists of, or is, the sequence of any one of primers set forth in Tables 1-3.

In certain embodiments, the primer comprises a “target annealing sequence” which comprises a sequence of any one of primers set forth in Tables 1-3, and a non-influenza virus A sequence.

The target annealing sequence will hybridize to at least a portion of a target nucleic acid in a sample, the target nucleic acid being homologous to, complementary to, transcribed or reverse transcribed from, or otherwise derived from, an influenza A HA. Thus, the target annealing sequence may also include flanking sequences encoded by or complementary to the sequence of the HA gene flanking the sequence defined by any one of primers set forth in Tables 1-3. The target annealing sequence may alternatively consist essentially of, or consist of, a sequence of primers set forth in Tables 1-3.

The non-influenza A virus sequence is a sequence that is not derived from or corresponding or complementary to the influenza A viral genome sequence. As described above, the non-influenza A virus sequence may be a sequence, for example, that is recognised by a protein or an enzyme, for example a restriction enzyme, or that is complementary to a nucleic acid sequence that is used for detection, for example, that is complementary to a probe that may be labelled or to a capture sequence of an immobilized nucleic acid molecule that may be used to capture the present primer. The non-influenza A virus sequences may be located 5′ to, 3′ to, or may flank on either side, the target annealing sequence.

The length of the primer or primers of the invention will depend on the desired use or application. For example, as will be understood, a PCR primer will typically be between about 15 and about 35 bases in length. The length of a PCR primer will be based on the sequence that is to be amplified as well as the desired melting temperature of the primer/template hybrid. However, for applications such as Southern hybridizations, the primer may be longer, for example from about 15 bases to about 1 kilobase in length or longer. Thus, the primer may be from 15 bases to about 1 kilobase in length, from 15 to about 500 bases, from 15 to about 300 bases, from 15 to about 150 bases, from 15 to about 100 bases or from 15 to 50 about bases.

The primers of the invention may be prepared using conventional methods known in the art. For example, standard phosphoramidite chemical ligation methods may be used to synthesize the primer in the 3′ to 5′ direction on a solid support, including using an automated nucleic acid synthesizer. Such methods will be known to a skilled person.

Although the term “primer” is used herein to describe single-stranded nucleotides that are used to anneal in a sequence-specific manner to a template sequence and initiate a new strand synthesis, a skilled person will understand that uses of the primers of the invention are not so limited. For example, the primers of the invention may be used as probes, to detect a complementary sequence to which the probe hybridizes. For such a use, the primer will typically be labelled for detection, for example, with a fluorescent label, a chemiluminescent label, a coloured dye label, a radioactive label, a protein including an enzyme, a peptide or a ligand for example biotin. When used as probes, the primers may be used in nucleic acid hybridization methods, single stranded conformational polymorphism (SSCP) analysis, restriction fragment polymorphism (RFLP) analysis, Southern hybridization, northern hybridization, in situ hybridization, electrophoretic mobility shift assay (EMSA), nucleic acid microarrays, and other methods that are known to those skilled in the art.

The primers of the invention may be used to diagnose or detect peptide epitopes of influenza H1-5, preferably H1, H3 and H5 in a sample, for example a biological sample derived from an organism suspected of carrying the virus.

Thus, there is provided a method for detecting peptide epitopes of influenza subtype H1-5 in a sample comprising amplifying DNA reverse transcribed from RNA obtained from the sample using one or more reverse primers comprising any one of the sequences set forth in Tables 1-3 and one or more forward primers comprising any one of the sequences set forth in Tables 1-3, and detecting a product of amplification, wherein the product indicates the presence of peptide epitope of an influenza virus subtype in the sample. Table 1 depicts primers for H1, Table 2 depicts primers for H3, and Table 3 primers for H5. It is not excluded that certain primers can bind to at least 2 subtypes, preferably to 3 subtypes. These primers can comprise 1, 2 or 3 degenerate nucleotides so that cross subtype identification is possible. Preferably a primer comprises 3 degenerate nucleotides, more preferably 2, even more preferably 1 and most preferably no degenerate primers.

The primers directed to one subtype can be used also as mixtures. This primer mixture can comprise at least 3 primers 2 of which can bind different subtypes and one binds both subtypes. The mixture can comprise 4 primer directed to 3 different binding sites in subtypes and one common binding site. Alternatively, 2 primer pairs can detect 2 HA subtypes. Moreover, mixtures can comprise multiple primers, for example, some primers can be directed to specific peptide epitopes of the present invention while other primers detect the whole HA gene or other specific peptide epitopes. The primers set forth in tables 1-3 can be mixed and skilled artisan understands how to mix the primers and take into account their Tm and other parameters.

Skilled artisan understands that primers can be used also separately. Primer pairs can be used alone and the data from each test or experiment can be combined. For example, one primer (pair) can detect the whole HA subtype and other pairs in other test chambers or vessels can identify peptide motifs. By these means identity of HA subtype can be obtained by combining the data from separate, but alternatively simultaneous or subsequent, tests or experiments.

There is also provided a method for detecting peptide epitopes of influenza subtype in a sample comprising amplifying DNA reverse transcribed from RNA obtained from the sample using one or more reverse primers comprising any one of the sequences set forth in Tables 1-3 and one or more forward primers comprising any one of the primers set forth in Tables 1-3, or using one or more reverse primers comprising any one of the primers set forth in Tables 1-3 and one or more forward primers comprising any one of the primers set forth in Tables 1-3, and detecting a product of amplification, wherein the product indicates the presence of a peptide epitope of an influenza virus subtype in the sample.

The term “detecting” an amplification product is intended to include determining the presence or absence, or quantifying the amount, of a product resulting from an amplification reaction that used template, primers, and an appropriate polymerase enzyme.

Typically, RNA from a sample is reverse transcribed so as to provide a single DNA strand that is complementary to the RNA HA gene. The reverse transcribing is performed using a reverse transcriptase enzyme that is capable of reading an RNA template and synthesizing a complementary DNA strand from a primer that binds to the RNA template, by polymerizing DNA nucleotides in a sequence complementary to that of the RNA template. Reverse transcriptase enzymes, for example T7 reverse transcriptase, are commercially available, and will be known to a skilled person. The reverse transcription reaction is typically performed in a buffer, under reaction conditions and at a temperature that are designed to optimize the reverse transcriptase activity. Commercially supplied reverse transcriptase enzymes may be supplied with a suitable buffer and DNA nucleotides.

The primer used in the reverse transcription reaction may be a mixture of random hexamers that will bind to random sites along the RNA template. Alternatively, the reverse transcription primer may be a specific primer designed to bind at a particular site within the HA gene gene. Therefore, one or more reverse primers comprising any one of primers set forth in Tables 1-3, may be used as a primer in the reverse transcription reaction. The same reverse primer or primers of the invention may be advantageously used in the amplification step, particularly when the reverse transcription and amplification are effected in the same reaction. Where more than one primer of the invention is used, each of the primers used will have a different sequence, the sequence of each primer comprising any one of primers set forth in Tables 1-3.

Where there is a family of primers based on the same conserved region of the HA gene but varying at one or more nucleotides within the primer sequence, for example ID F NO: 22 to ID F NO: 24, one or more reverse primers from such a family may be used. This allows for reverse transcription of, and therefore eventual detection of, a wide number of possible isolates or variants of influenza virus subtype. A “variant” as used herein refers to an HA subtype in which the HA gene sequence may vary from that of another HA subtype, or an HA subtype in which the HA gene sequence may vary from that of another HA subtype.

The template RNA for the reverse transcription reaction may be obtained from a sample using RNA extraction methods known in the art. RNA extraction kits are also commercially available, for example, RNeasy™ kits (Qiagen), and the availability and use of such kits will be known and understood by a skilled person.

The sample may be a biological sample, for example any sample collected from an individual suspected of carrying influenza virus subtype. The sample may be any sample that contains the virus from an infected individual, and includes tissue and fluid samples, for example, blood, serum, plasma, peripheral blood cells including lymphocytes and mononuclear cells, sputum, mucous, urine, feces, throat swab samples, dermal lesion swab samples, cerebrospinal fluids, pus, and tissue including spleen, kidney and liver.

The forward primers directed against HA gene of influenza A virus subtypes are any of sequences of ID F NOS: 1-60, ID F NOS: 91-150 and ID F NOS: 195-257. A skilled person will understand that the forward and reverse primers used in a particular amplification reaction need to correspond with respect to subtype and gene. Therefore, when a reverse primer is used that comprises any one of ID R NO: 61 to ID R NO: 90, a forward primer may be used that comprises any one of ID F NO:1 to ID F NO:60. Similarly, when a reverse primer is used that comprises any one of ID R NO: 151 to ID R NO: 194, a forward primer may be used that comprises any one of ID F NO: 91 to ID F NO: 150. Similarly, when a reverse primer is used that comprises any one of ID R NO: 258 to ID R NO: 306, a forward primer may be used that comprises any one of ID F NO: 195 to ID F NO: 257. Alternatively, by using degenerate primers in well conserved region, like ID F NO: 12 or ID F NO: 95, two forward primers can be replaced with one, and only two additional reverse primers are needed, for example, ID R NO: 89 and ID R NO: 170.

One or more reverse primers may be chosen from primers comprising ID R NO: 61 to ID R NO:90 or ID R NO: 151 to ID R NO: 194 or ID R NO: 258 to ID R NO: 306, and one or more forward primers may be chosen from primers comprising ID F NO: 1 to ID F NO:60, ID F NO:91 to ID F NO:150, or ID F NO: 195 to ID F NO: 257 even where the primers do not fall within a family of primers. However, this will result in a series of amplification of products of varying lengths. If the multiple reverse and/or forward primers are carefully chosen, amplification products may be readily distinguishable from each other. It should be noted that in this embodiment, the sensitivity of the detection method may be reduced, yielding less of a particular amplification product from a given amount of template. As in the reverse transcription reaction, where more than one primer of the invention is used each of the primers used will have a different sequence, the sequence of each primer comprising any one of ID R NO:61 to ID R NO:90, ID R NO:151 to ID R NO: 194, or ID R NO: 258 to ID R NO: 306 for the reverse primers and any one of ID F NO:1 to ID F NO:60, ID F NO:91 to ID F NO:150, or ID F NO: 195 to ID F NO: 257 for the forward primers.

The forward primer is chosen such that in combination with the reverse primer used, a detectable double-stranded DNA amplification product is produced. That is, the forward primer should be located sufficiently upstream in the HA gene relative to the reverse primer to amplify a double stranded DNA molecule that is of sufficient size such that when produced in the amplification reaction, it is capable of being detected by whichever detection method is chosen. The size of DNA product that can be detected will vary with the specific detection method chosen. For example, if agarose gel electrophoresis is used to detect the amplification product, the end product may have to be larger than if real time PCR using lightcycling is used as the detection method. Depending on the concentration of gel used, agarose gel electrophoresis can be used to detect fragments as small as 25 base pairs. However, larger fragments, for example between 150 to 500 base pairs, are more readily detected using gel-based methods, whereas smaller fragments, for example, less than 100 base pairs are easily detected using real time PCR methods.

The amplified DNA product may be detected using detection methods known in the art. For example, suitable detection methods include, without limitation, incorporation of a fluorescent, chemiluminescent or radioactive signal into the amplified DNA product, or by polyacrylamide or agarose gel electrophoresis, or by hybridizing the amplified product with a probe containing an electron transfer moiety and detecting the hybridization by electronic detection methods.

The detection method may be performed subsequent to the amplification reaction. Alternatively, the detection method may be performed simultaneously with the amplification reaction. In one embodiment, the amplified DNA product is detected using real time PCR, for example by lightcycling, for example using Roche's LightCycler™ Real time PCR techniques will be known by a skilled person and may involve the use of two probes each labelled with a specific fluorescent label, and which bind to the amplified DNA product. The probes are designed such that they bind to the DNA product in such a manner that the fluorescent label of the first probe is in close proximity to the fluorescent label of the second probe. The amplification reaction is performed in an instrument designed to emit and detect the relevant fluorescent signals, and includes an additional detection segment in which the instrument emits light at a wavelength suitable to excite the fluorescent label on the first probe, which then emits light at a wavelength suitable to excite the fluorescent label on the second probe. The light which is then emitted by the second probe's fluorescent label, and which differs in wavelength from the previous emissions, is detected by the instrument.

Alternatively, a fluorescent molecule that binds to double stranded DNA may be used where a single stranded template is used in the amplification reaction. This method allows for detection and fairly precise relative quantification, when compared with a known standard template, of the amplified DNA product throughout the amplification reaction. The quantification of amplified product may enable the determination of viral load in the original biological sample. As well, this method allows for the detection of smaller amounts of amplification products, and amplification products having smaller sizes than methods using conventional PCR techniques.

The simultaneous amplification and detection may also be performed using a detection probe that is labelled at the 5′end with a fluorophore and at the 3′ end with a quenching molecule that quenches emissions of the fluorophore when in proximity to the fluorophore, as in the Taqman™ method designed by ABI Systems. The detection probe will bind to the forward or reverse strand of the amplification template. A polymerase having 5′ exonuclease activity, for example, Taq polymerase or others (for example, synthetic version is available from Roche), is used in the amplification reaction. As the template strand having the bound detection probe is amplified, the detection probe will be digested by the 5′ exonuclease, removing the fluorophore from the proximity of the quencher and allowing the fluorophore to emit. The emissions can be quantified in standard equipment, for example, the LightCycler™ described above.

Although the above embodiments have been described in the context of a PCR amplification method, a skilled person will understand that the sequences of the invention may be used to design primers for use in other amplification methods to detect human or other species influenza virus subtypes in a biological sample. For example, the sequences disclosed in ID F/R NO: 1 to ID F/R NO: 306 may be used to design primers for amplification and detection by NASBA methods, as described for example in Lau et al. (Biochem. Biophys. Res. Comm. 2003 313:336-342), and which are generally known to a skilled person.

Briefly, in the NASBA technique the primers are designed to bind to a portion of the gene of interest, here HA or NA, and to include a promoter for an RNA polymerase, for example T7 RNA polymerase. The viral gene is reverse transcribed and a second complementary DNA strand is synthesized to produce a double stranded DNA molecule that includes an intact RNA polymerase promoter. The relevant RNA polymerase is used to generate copies of an RNA molecule corresponding to an amplified portion of the gene of interest. The amplified RNA is then bound to a detection molecule, typically a nucleic acid that is complementary to a portion of the amplified RNA and that is labelled, for example, with a radio label, a chemiluminescent label, a fluorescent label or an electrochemiluminescent label. The amplified RNA bound to the detection molecule is then typically captured by a capture molecule, for example an immobilized nucleic acid that is complementary to a portion of the amplified RNA product that is a different portion than that to which the detection molecule binds. The captured RNA amplification product with bound detection molecule is then detected by the relevant detection method as determined by the label on the detection molecule and the method of capture.

Thus, the present invention contemplates the use of a primer comprising any one of ID F/R NO: 1 to ID F/R NO: 306 for use in NASBA methods to detect the presence of influenza virus subtype H1-5 in a biological sample.

The primers of the invention are also useful for sequencing a DNA molecule corresponding to the HA gene, or a reverse transcribed DNA molecule complementary to the HA gene of the influenza virus subtype H1, H2, H3, H4, H5 and/or H6-16. A reverse primer comprising any one of ID R NO: 61 to ID R NO: 90, or any one of ID R NO:151 to ID R NO: 194, or any one of ID R NO: 258 to ID R NO: 306 may be used to initiate a sequencing reaction using as template nucleic acid molecule corresponding to a portion of the HA gene, respectively. A forward primer comprising any one of ID F NO: 1 to ID F NO: 60 or any one of ID F NO: 91 to ID F NO: 150 or any one of ID F NO: 195 to ID F NO: 257 may be used to initiate a sequencing reaction using as template a nucleic acid molecule complementary to a portion of the HA gene, respectively. Sequencing reactions may be performed using standard methods known in the art, and may be performed using automated sequencing equipment.

The primers of the invention are also useful as probes or capture molecules to detect RNA from an H1-5 influenza virus isolate. For example, one or more primers comprising any one of ID F/R NO: 1 to ID F/R NO: 306 may be immobilized on a solid support and used to isolate nucleic acid molecules having a sequence that is complementary to some or all of the primer sequence.

Thus, there is presently provided a method for detecting influenza A virus peptide epitopes in a sample comprising contacting one or more immobilized primers comprising any one of the sequences of ID F/R NO: 1 to ID F/R NO: 306 with the sample.

The primer may be immobilized on a solid support using standard methods for immobilizing nucleic acids, including chemical cross-linking, photocross-linking, or specific immobilization via a functional group on the primer, including a functional group that is added to or incorporated into the primer, for example biotin.

The solid support may be any support which may be used in a detection assay, including chromatography beads, a tissue culture plate or dish, or a glass surface such as a slide.

One example of an immobilization and capture application is incorporation of the primer or primers in a DNA or nucleotide microarray, as is known in the art.

Thus, there is also provided a method of detecting influenza A virus subtype peptide epitopes in a sample comprising contacting a microarray containing one or more primers comprising any one of the sequences of ID F/R NO: 1 to ID F/R NO: 306 in at least one spot in the microarray with the sample, and detecting hybridization of the sample to the primer. Nucleic acid microarray technology is known in the art, including manufacture of a microarray and detection of hybridization of a sample with the capture molecules in one or more spots in the microarray.

The present invention contemplates an isolated nucleotide encoding an antigenic compound according to any one of claims 1-21. Nucleotides encoding an antigenic compound are useful in applications where specific type of an HA subtypes is determined. It is understood that degenerate nucleic acid sequences encode the same amino acid sequence.

The invention is directed to methods for detecting nucleic acid encoding antigenic compound according to claim 1 in a sample comprising:

amplifying DNA reverse transcribed from RNA obtained from the sample using one or more primers each comprising a sequence of any one of ID F/R NO: 1 to ID F/R NO: 306 or sequences in FIGS. 17-19;
and detecting a product of amplification, wherein the presence of the product of amplification indicates the presence of an influenza virus hemagglutinin in the sample.

When specific primers are selected, type of the HA is also determined.

The primers can essentially consist of any one of the sequences of ID F/R NO: 1 to ID F/R NO: 306 or sequences set forth in FIGS. 17-19. Or preferably, a primer of claim is any one of the sequences of ID F/R NO: 1 to ID F/R NO: 306 or sequences set forth in FIGS. 17-19.

The method preferably amplifies comprising using a primer set, the primer set comprising

(a) one or more reverse primers each comprising a sequence of any one of ID R NO:61 to ID R NO:90, and one or more forward primers each comprising a sequence of any one of ID F NO:1 to ID F NO:60 or sequences set forth in FIGS. 17-19, or
(b) one or more reverse primers each comprising a sequence of any one of ID R NO:151 to ID R NO:194, and one or more forward primers each comprising a sequence of any one of ID F NO: 91 to ID F NO: 150 or sequences set forth in FIGS. 17-19, or
(c) one or more reverse primers each comprising a sequence of any one of ID R NO: 258 to ID R NO: 306 and one or more forward primers each comprising a sequence of any one of ID F NO:195 to ID F NO:257 or sequences set forth in FIGS. 17-19;
wherein the presence of the product of amplification indicates the presence of an influenza virus hemagglutinin in the sample. The presence of influenza virus HA also indicates the amino acid composition of an antigenic compound present in HA subtype(s). It is useful to know what antigenic compound is as an animal subject can be vaccinated with the corresponding antigenic compound or an antibody substance.

The above method can further comprise the step of reverse transcribing RNA obtained from the biological sample using one or more reverse primers each comprising a sequence of any of ID R NO:61 to ID R NO:90, ID R NO:151 to ID R NO:194, and ID R NO:258 to ID R NO:306 or sequences set forth in FIGS. 17-19.

The sequences of one or more reverse primers each has a sequence of: ID R NO:61 to ID R NO:90, ID R NO:151 to ID R NO:194, and ID R NO:258 to ID R NO:306 or sequences set forth in FIGS. 17-19.

The preferred method comprises one or more forward primers each has the sequence of: ID F NO:1 to ID F NO:60; ID F NO: 91 to ID F NO: 150 and ID F NO: 195 to ID F NO: 257 or sequences set forth in FIGS. 17-19.

In a preferred embodiment amplifying comprises amplifying by PCR amplification or real time PCR. Detection step preferably comprises detecting by an agarose or acrylamide gel.

In an alternative method nucleic acid encoding antigenic compound according to claim 1 is detected in a sample comprising contacting the sample with a primer immobilized on a support, said primer comprising a sequence of any one of ID F/R NO: 1 to ID F/R NO: 306 or sequences in FIGS. 17-19, under conditions suitable for hybridizing the primer and the sample; and detecting hybridization of the primer and the sample.

The primers consists essentially of any one of the sequences of ID F/R NO: 1 to ID F/R NO: 306 or sequences in FIGS. 17-19. Or primer is any one of the sequences of ID F/R NO: 1 to ID F/R NO: 306 or sequences in FIGS. 17-19.

In another embodiment, nucleic acids encoding antigenic compound according to claim 1 in a sample comprising: contacting the sample with a nucleic acid microarray, the nucleic acid microarray comprising one or more primers, each of said primers comprising a sequence of any one of ID F/R NO: 1 to ID F/R NO: 306 or sequences in FIGS. 17-19, under conditions suitable for hybridizing the one or more primers and the sample; and detecting hybridization of the one or more primers and the sample.

The one or more primers in the above method consists essentially of any one of the sequences of ID F/R NO: 1 to ID F/R NO: 306 or sequences in FIGS. 17-19. Or one or more primers is any one of the sequences of ID F/R NO: 1 to ID F/R NO: 306 or sequences in FIGS. 17-19.

A nucleic acid microarray comprising a primer, said primer comprising a sequence of any one of ID F/R NO: 1 to ID F/R NO: 306 or sequences in FIGS. 17-19. One or more primer consists essentially of any one of the sequences of ID F/R NO: 1 to ID F/R NO: 306 or sequences in FIGS. 17-19. Or the primer is any one of the sequences of ID F/R NO: 1 to ID F/R NO: 306 or sequences in FIGS. 17-19.

The invention also contemplates a kit comprising a primer and/or nucleic acid according to any one of claims 27 to 49 and instructions for detecting antigenic compound according to claim 1. The kit is useful to detect efficiently an antigenic compound or compounds of the present invention.

Invention encompasses also a primer comprising a sequence of any one of ID F/R NO: 1 to ID F/R NO: 306 and in FIGS. 17-19. The primer can consist essentially of any one of the sequences of ID F/R NO: 1 to ID F/R NO: 306 and in FIGS. 17-19. Or primer is any one of the sequences of ID F/R NO: 1 to ID F/R NO: 306 and in FIGS. 17-19.

The amino acid sequence and 3D-structure of influenza X-31 hemagglutinin is described previously, e.g., in PCT/FI2006/050157 (published as WO2006111616).

### EXAMPLES

### Example 1

### Modeling Studies of the Influenza Hemagglutinin

Introduction—The X-ray crystallographic structure of the hemagglutinin of the X-31 strain of human influenza virus was used for the docking (PDB-database,www.rcsb.org/pdp, the database structure 1HGE). The structure used in the modelling is a complex structure including Neu5Acα-OMe at the primary sialic acid binding site, the large oligosaccharide modelled to the site had one Neu5Aα-superimposable to the one in the 1HGE, but glycosidic glycan instead of the methylgroup. The studies and sequence analyses described below in conjunction with hemagglutination-inhibition studies used for evaluation of the binding efficacy of the different branched poly-N-acetylactosamine inhibitors. The basic hemagglutinin structure consists of a trimer comprising the two subunits HA1 and HA2, the first of which contains the primary sialic acid binding site.

In addition to the primary site, which binds to both sialyl-α3-lactose and sialyl-α6-lactose, a secondary site exists which has been previously found to bind sialyl-α3-lactose as well but not sialyl-α6-lactose.

Results—Docking of the best binding inhibitory structures was performed under the premise that the primary sialic acid site of the hemagglutinin serves as the nucleation point from which the rest of the oligosaccharide folds itself onto the protein surface. From previous crystal structures of various complexes with small linear oligosaccharides and a branched structure it was obvious that maximally three sugars could be accommodated within the primary site and that further sugars will force the oligosaccharide to fold itself in different directions outside the primary site depending on the actual structure. The only structurally relevant branched compound investigated so far is

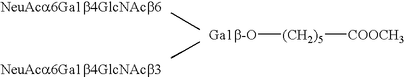

for which only the three terminal sugars of one of the branches is visible in the crystal structure and where the GlcNAc residue is seen to double back placing it on top of the NeuAc residue.

Of the various branched type 2-based disialylated oligosaccharides produced by Carbion for testing of their inhibitory power in the hemagglutination assay, two structures stood out for clearly stronger binding effectivity than the other isomers of similar size: and

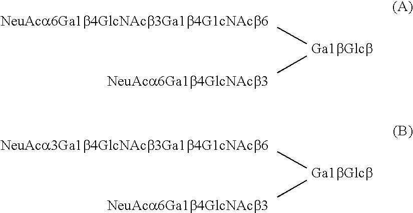

For these larger branched disialylated oligosaccharide structures the topography of the protein surface, the distribution of mutations of residues noncritical for binding from a large number of strains (see below) as well as the existence of a secondary site located within reach of the structures in question, suggested an oligosaccharide fold that would have to involve both the primary and secondary sites and that as a further prerequisite the NeuAc residue in the primary site would have to be α6-linked.

With these considerations in mind it was found that the two structures given above could be manually docked into both the primary and secondary sites without building any strain into either the oligosaccharides or the protein structure, meaning that only energetically favorable conformations around the constituent disaccharide glycosidic linkages as documented earlier in the literature had to be employed. Ensuing energy minimizations and dynamics simulations of these two complexes yielded the pictures shown below.

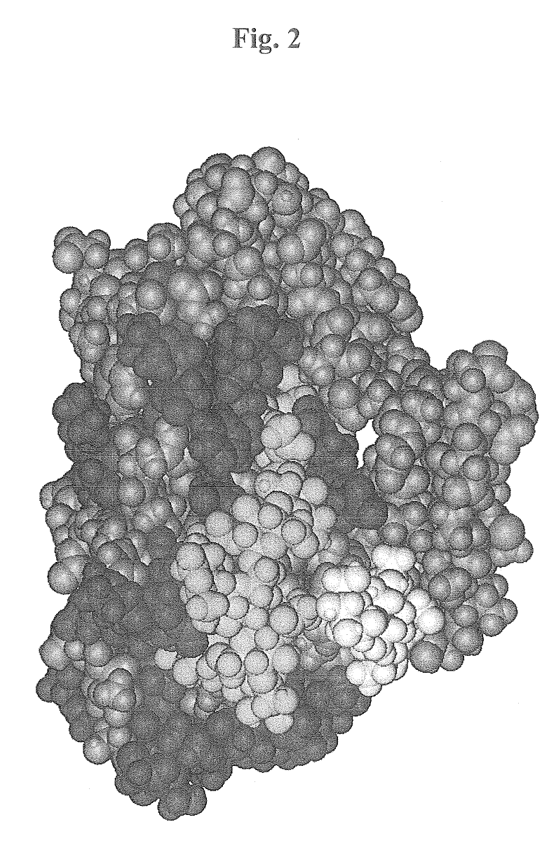

In the FIG. 2 the oligosaccharide having both NeuAc residues α6-linked is shown with the sialic acid of the shorter branch in the primary site at the top of the protein and the other sialic acid at the bottom in the pocket of the secondary site. Although the sialic acid interacts with some amino acid side chains that are identical to those found in the NeuAcα3Galβ4Glc complex an exact superposition cannot be attained since the oligosaccharide is in its most extended conformation leaving the NeuAcα6 residue 2-3 Å above the corresponding NeuAcα3 residue of the trisaccharide. Regarding the oligosaccharide having a NeuAcα3 residue attached at the longer branch a very similar picture is arrived at except of course for the sialic acid itself (not shown). It is noteworthy that the NeuAcα3 residue could be accommodated in the binding pocket without any repositioning of the oligosaccharide chain or perturbation of the protein structure, suggesting that the docked structures may be close to the actual complexes.

Further evidence for the probability of the docked structures being relevant for the true complexes comes from comparative hemagglutination-inhibition studies using structure (B) and different strains of the virus.

| | | Hemagglutination-inhibition |
|---|---|---|
| Virus strain | Hemagglutination | using structure (2) at 5 mM |
| A/Aichi/68 (X:31) | ++ | − |
| A/Victoria/3/75 | ++ | − |
| A/Japan/305/57 | ++ | ++ |
| A/Hong Kong/8/68 | ++ | − |
| A/PR/8/34 | ++ | + |
| B/Lee/40 | ++ | − |

As can be seen the A/Japan/305/57 and A/PR/8/34 strains are not inhibited by structure (B) whereas the other strains are completely inhabitable. A sequence comparison between these strains reveals interesting mutations at critical positions which further substantiates the proposed structure of this complex. First of all, any mutations around the primary site are expected to affect hemagglutination and hemagglutination-inhibition equally whereas mutations occurring further along the oligosaccharide chain towards or in the secondary site are expected to affect the hemagglutination-inhibition only. Secondly, mutations at various positions in strains which are completely inhabitable can be discarded as being important for binding. With this line of reasoning at least three mutations at positions 100, 102 and 209 could be identified in both strain A/Japan/305/57 and strain A/PR/8/34 relative to A/Aichi/68 (X:31) and which are localized around the terminal NeuAcα3 in the deepest part of the secondary binding site. The first two mutations are sterically compensatory in nature (Y100G and V102F, identical for both strains) while the third mutation (S209L in A/Japan/305/57 and S209Y in A/PR/8/34) introduces an even more hydrophobic environment than before. Especially the V102F mutation is expected to affect binding strongly since the phenylalanine side chain would come in contact with the sialic acid carboxyl group in the present model

The sequence analysis was carried further by scanning the SwissProt and TREMBL data bases for the 100 most homologous sequences relative to A/Aichi/68 (X:31). By indicating all mutations occurring in these strains by color one gets a view of where on the surface of the hemagglutinin the antigenic drift has been most prevalent in order for the virus to elude the host immune response, and even though it is likely that several of these species-specific strains have different binding specificities the invariant or conservatively mutated regions on the hemagglutinin surface can be regarded as good candidates for ligand interactions. Below three different views of the oligosaccharide binding region is shown with and without the oligosaccharide.

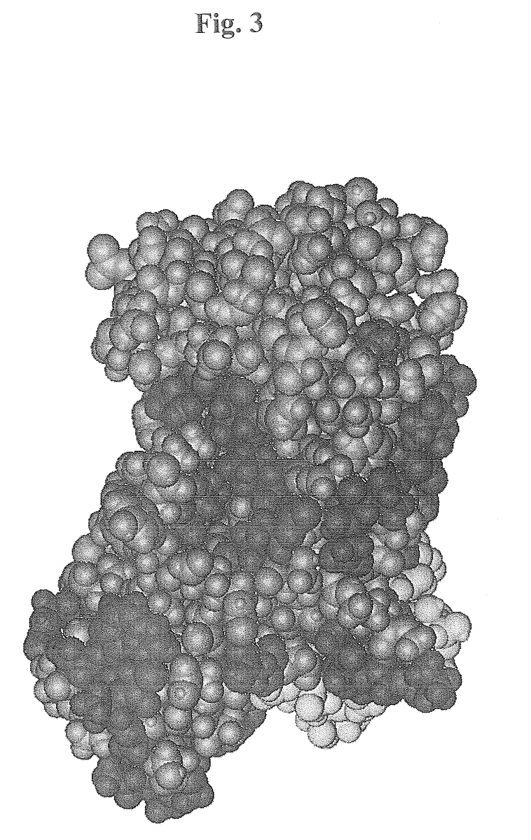

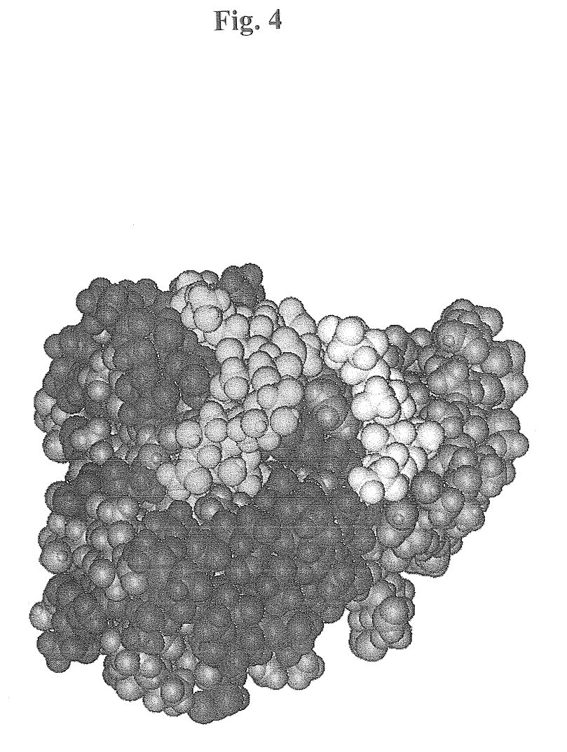

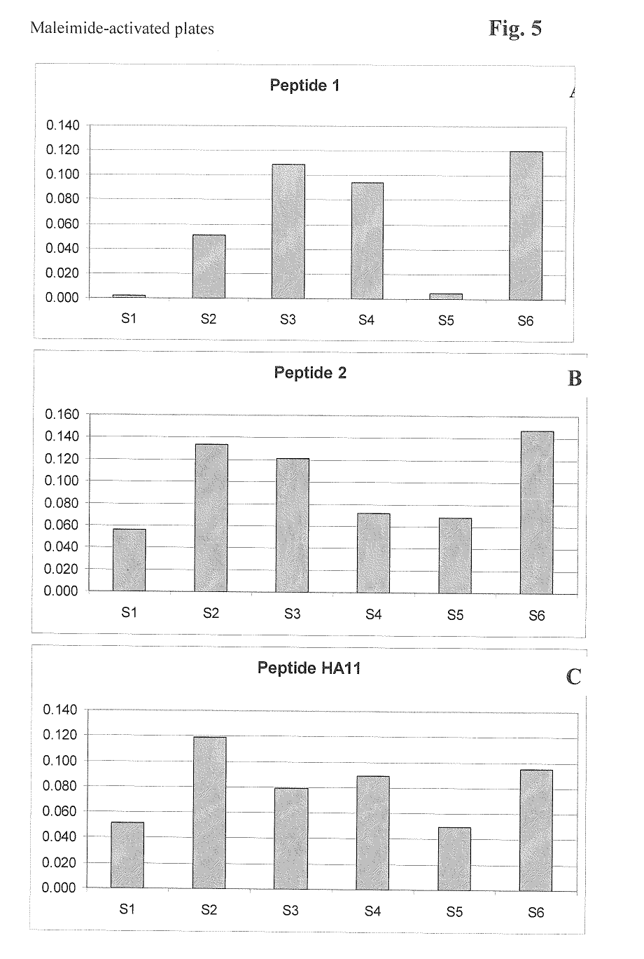

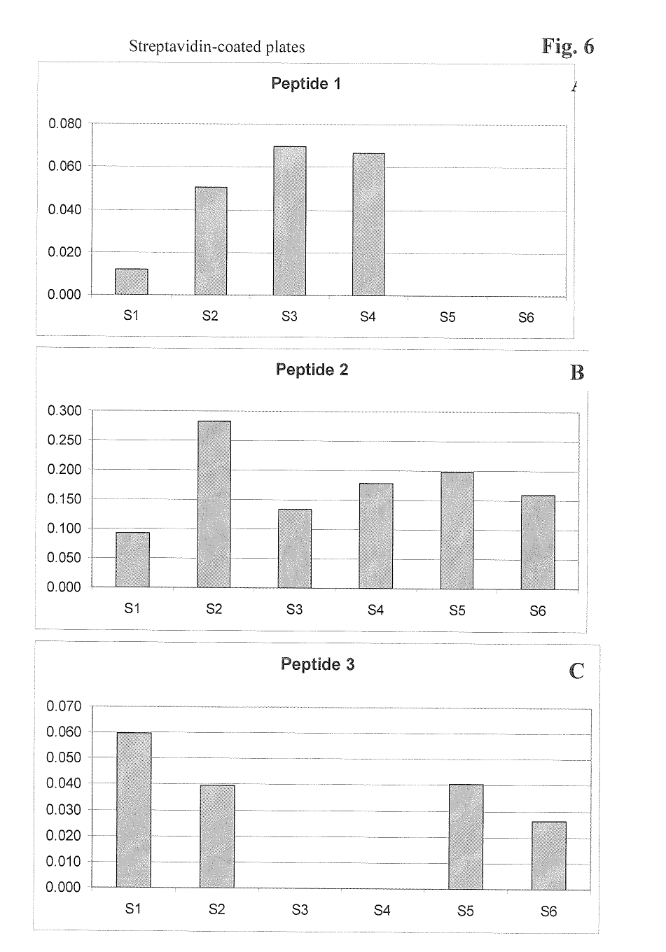

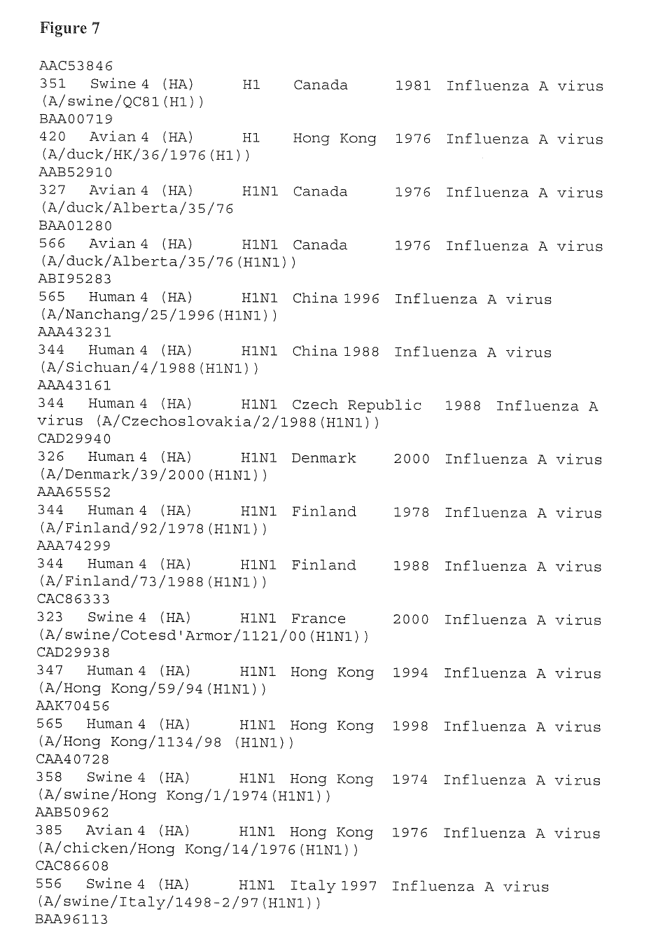

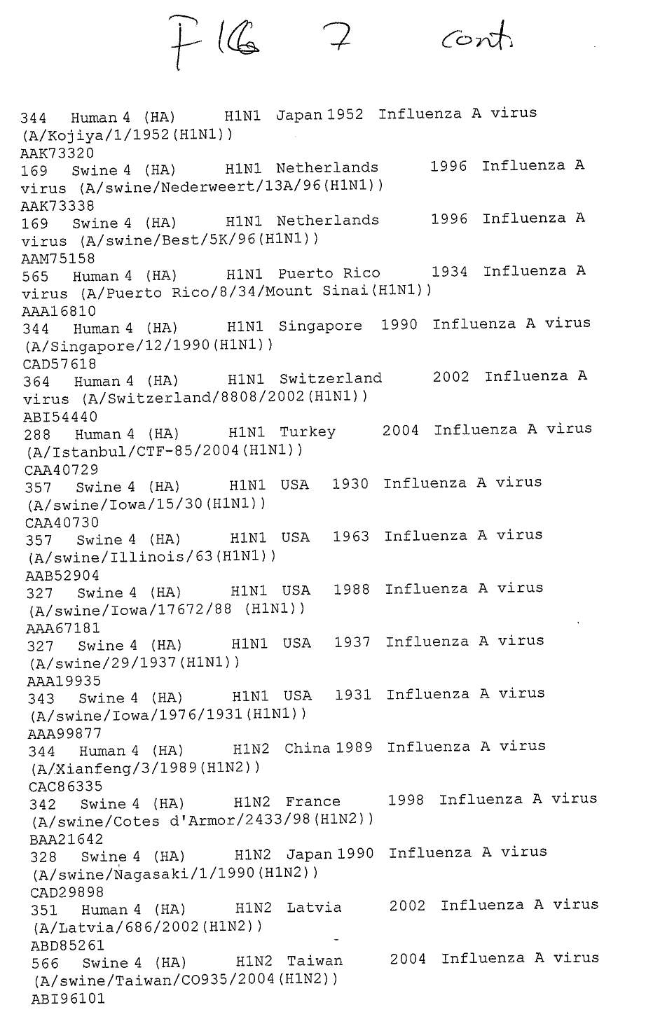

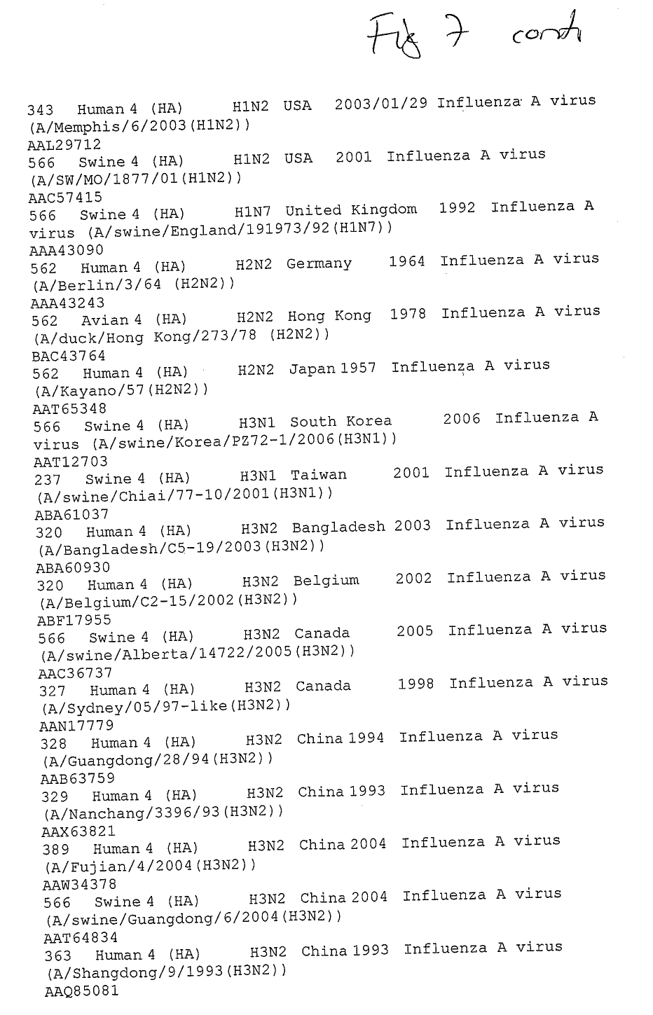

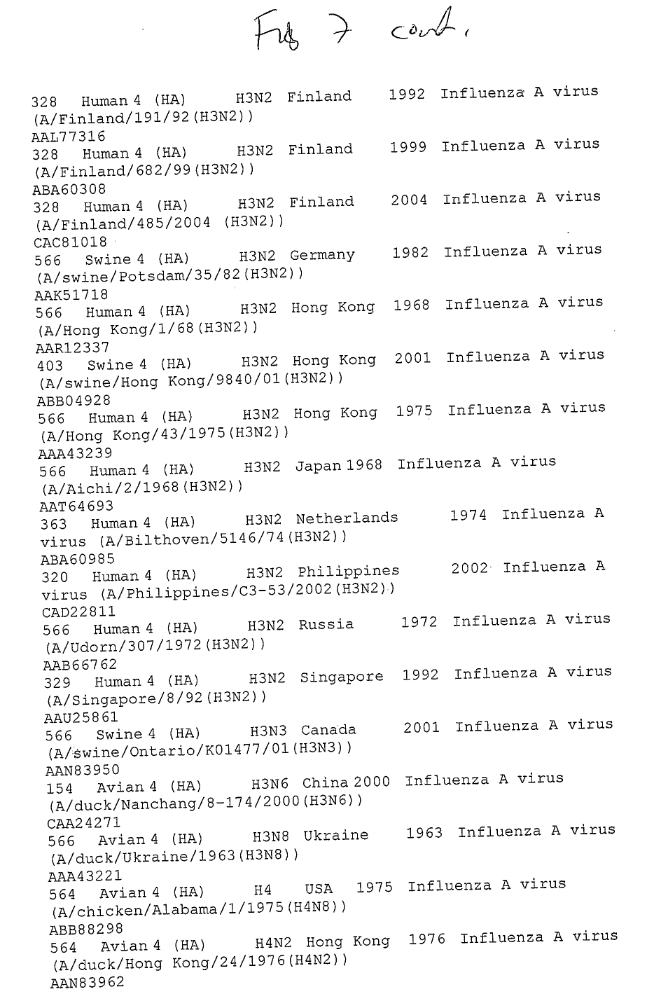

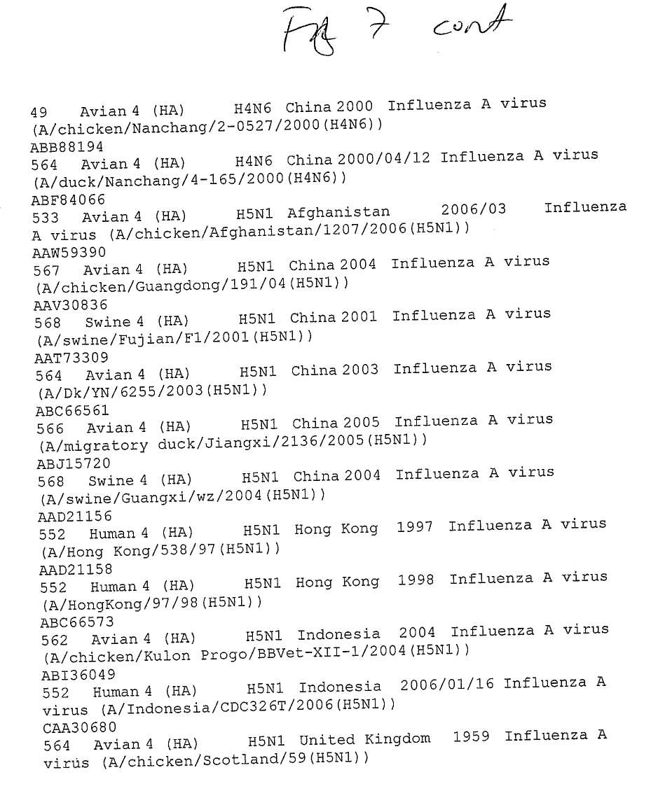

The panels, FIG. 5, shows a “front” view while the panels in FIG. 4 and in FIG. 3 show “right side” and “top” views, respectively. Mutations are colored red and the N-linked sugars are in white whereas the oligosaccharide is shown in yellow. It is evident that the highest mutational frequencies are found on the protruding parts of the protein surface which also are the ones most readily accessible for antibody interactions. The primary site is mainly blue and thus highly conserved as expected as is the path halfway down to the secondary site. However, most of the mutations seen at positions to the lower left of the oligosaccharide point away from the sugars and the mutations to the lower right of the sugars in most cases are conservative or otherwise nondestructive with regard to the secondary binding site topology.

The Complex Structure and Interactions of Oligosaccharide Ligand with the Influenza Virus

Table 1 shows the interactions of the primary site with the saccharide A (oligosaccharide structure 7 according to the Table 3) in complex structure show in FIG. 2. The primary site is referred as Region A, the bridging site referred as region B and the secondary site is referred as Region C. The conserved amino acid having interactions with the oligosaccharide structures are especially preferred according to the invention. The data contains also some semiconservative structures which may mutate to similar structures and even some nonconserved amino acid structures. The nonconserved amino acids may be redundant because their side chains are pointing to the opposite direction. Mutations of the non-conserved or semiconserved amino acid residues are not expected to essentially chance the structure of the large binding site. VDW referres to Van Der Waals-interaction, hb to hydrogen bond. The Table 1 also includes some interactions between amino acid residues in the binding site.

The Table 2 shows the torsion angles between the monosaccharide residues according to the FIG. 1. Glycosidic dihedral angles are defined as follows: phi=H1-C1-O1-C′X and psi=C1-O1-C′X-HX for 2-, 3- or 4-linked residues; phi=H1-C1-O1-C′6, psi=C1-O1-C′6-C′5 and omega=O1-C′6-C′5-O′5 for a 6-linked residue. Imberty, A., Delage, M.-M., Bourne, Y., Cambillau, C. and Pérez, S. (1991) Data bank of three-dimensional structures of disaccharides: Part II. N-acetyllactosaminic type N-glycans. Comparison with the crystal structure of a biantennary octasaccharide. Glycoconj. J., 8, 456-483. The torsion angles define conformation of oligosaccharide part in the complex structure.

### Additional Modelling Work

Distances between carboxylic acid groups of sialic acid residues in binding conformation were produced with X31-hemagglutinin model. The large divalent saccharide 25 with two α6-sialylpentasaccharides had an extended length (most likely conformation with regard to glycosidic torsion angles) of about 59 Å and it could be docked to the primary and secondary sites, the saccharide 26 had an extended length of 47 Å and it could not be docked both to primary and secondary site, the saccharide 27 had extended length of 36 Å and could be fitted to both primary and secondary sites with a configuration similar to saccharide 17; and the saccharide 28 has the extended length of 49 Å with docking to both primary and secondary site.

### Example 2

### Materials and Methods for ELISA Assays of Peptides

### ELISA Assays on Maleimide-Activated Plates

Peptides containing cysteine were bound through the cysteine sulfhydryl group to maleimide activated plates (Reacti-Bind™ Maleimide activated plates, Pierce). The peptides sequences were as follows:

Biotin-aminohexanoyl-SYACKR (custom product, CSS, Edinburgh, Scotland)

Biotin-aminohexanoyl-SKAYSNC (custom product, CSS, Edinburgh, Scotland)

CYPYDVPDYA (HA11; Nordic Biosite)

All peptides were dissolved in 10 mM sodium phosphate/0.15 M NaCl/2 mM EDTA, pH 7.2, to a concentration of 5 nmol/ml. One hundred microliters of the peptide solution (0.5 nmol of peptide) was added to each well and allowed to react overnight at +4° C. The plate was then washed three times with 10 mM sodium phosphate/0.15 M NaCl/0.05% Tween-20, pH 7.2).

The unreacted maleimide groups were blocked with 2-mercaptoethanol: 150 μl of 1 mM 2-mercaptoethanol in 10 mM sodium phosphate/0.15 M NaCl/2 mM EDTA, pH 7.2 was added to each well and allowed to react for 1 hour at RT. The plate was then washed three times with 10 mM sodium phosphate/0.15 M NaCl/0.05% Tween-20, pH 7.2. The plate was further blocked with 1% bovine serum albumin (BSA) in 10 mM sodium phosphate/0.15 M NaCl/0.05% Tween-20, pH 7.2, and then washed with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 30 mins at RT. Control wells contained no peptide but both 2-mercaptoethanol and BSA blockings were employed. All incubations were performed in duplicates.

The plate was then washed with the washing buffer 8 times with at least 5 min incubation period between change of the washing liquid.

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+ color reagent (Dako Cytomation) was then added. The absorbance was read at 650 nm after 15 mins. Immediately after this measurement 100 μl of 1 M sulphuric acid was added and the absorbance read at 450 nm. Results are shown in FIG. 17.

### ELISA Assays on Streptavidin-Coated Plates

Biotinylated peptides were bound to streptavidin-coated plates (Pierce).

The peptides sequences were as follows:

Biotin-aminohexanoyl-PWVRGV (custom product, CSS, Edinburgh, Scotland)

Biotin-aminohexanoyl-SYACKR (custom product, CSS, Edinburgh, Scotland)

Biotin-aminohexanoyl-SKAYSNC (custom product, CSS, Edinburgh, Scotland)

Prior to peptide immobilization, plates were blocked with 150 μl of 0.5% BSA in 10 mM sodium phosphate/0.15 M NaCl/0.05% Tween-20, pH 7.2, for 1.5 h at RT. The plate was then washed three times with 10 mM sodium phosphate/0.15 M NaCl/0.05% Tween-20, pH 7.2.

Peptides were dissolved in 10 mM sodium phosphate/0.15 M NaCl, pH 7.2, to a concentration of 0.5 nmol/ml. One hundred microliters of the peptide solutions (50 μmol of the peptide) were added to the wells and allowed to react overnight at +4° C. The plates were then washed four times with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 60 mins at RT. Control wells did not contain peptides but were blocked as above. All incubations were performed in duplicates.

After serum incubation the plate was washed with the washing buffer 8 times with at least 5 min incubation period between change of the washing liquid.

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+color reagent (Dako Cytomation) was then added. The absorbance was read at 650 nm after 15 mins. Immediately after this measurement 100 μl of 1 M sulphuric acid was added and the absorbance read at 450 nm.

### Results of ELISA Assays of Antigen Peptides

### Design of the Experiments

Three antigen peptides were analysed against natural human antibodies from healthy adults. The individuals were selected based on the resistance against influenza for several years. The persons had been in close contact with persons with distinct influenza type disease in their families and/or at work but have not been infected for several years. At the time of blood testing two of the persons had influenza type disease at home but persons were suffering from only mild disease. The persons were considered to have good immune defense against current influenza strains.

The antigen peptides were selected to correspond structures present on recent influenza A (H3N2) strains in Finland (home country of the test persons). The assumption was that the persons had been exposed to this type of viruses and they would have antibodies against the peptides, in case the peptides would be as short linear epitopes effectively recognizable by human antibodies and peptide epitopes would be antigenic in human. The invention revealed natural human antibodies against each of the peptides studied. The data indicates that the peptides are antigenic and natural antibodies can recognize effectively such short peptide epitopes.

All antigen peptides 1-3 were tested as N-terminal biotin-spacer conjugates, which were immobilized on a streptavidin plate. Aminohexanoic acid spacer was used to allow recognition of the peptides without steric hindrance from protein. It is realized that the movement of the N-terminal part of peptide was limited, which would give conformational rigidity to the peptide partially mimicking the presence on a polypeptide chain.

The Peptides 1 and 2 were Also Tested on Maleimide Coated Plates.

The peptide 1 (Biotin-aminohexanoic-SKAYSNC) was also tested as conjugated from natural C-terminal Cys-residue in a antigen peptide, the peptide further contained spacer-biotin structure at amino terminal end of the peptide. The peptide presented natural C-terminal and Cys-linked presentation at C-terminus of the peptide presenting a preferred conformational structure. The presentation as natural like epitope was further supported by spacer structure blocking the N-terminus and restricting its mobility.

The peptide 2 (Biotin-aminohexanoic-SYACKR) was also tested as conjugated from natural Cys-residue in the middle of the antigen peptide. The peptide presented natural middle Cys-linked presentation at C-terminus of the peptide presenting a preferred conformational structure. The presentation as natural like epitope was further supported by spacer structure blocking the N-terminus and restricting its mobility.

### Control and Core Peptide

A commercial peptide CYPYDVPDYA (HM11-peptide), which has been used as a recognition tag on recombinant proteins was used as a control and for testing of analysis of binding between a free core peptide and human antibodies. Due to restricted availability of at least N-terminal sequence the peptide would not be very effective in immunization against the viral as therapy. This peptide is known to be antigenic in animals under immunization conditions and antibodies including polyclonals from rabbit, mice etc. The ELISA assay was controlled by effective binding of commercial polyclonal antibody from rabbit to the peptide coated on a maleimide plate, while negligible binding was observed without the peptide.

### Results

The absorbance was recorded by two methods (A450 and A650) and with three different dilutions giving similar results (the results with optimal dilutions giving absorbance values about 0.1 AU to about 0.8 AU and by absorbance at 450 nm are shown).

### Peptide 1 as Aminoterminal Conjugate and C-Terminal Cys-Conjugate

Biotin-aminohexanoic-SKAYSNC was tested against the 6 sera as N-terminal conjugate on a streptavidin plate. The sera 3 and 4 showed strongest immune response before serum 2, while sera 1, 5 and 6 were weakly or non-reactive against the construct.

The C-terminal cysteine conjugate of peptide 1 reacted with sera in the order from strongest to weaker: 6, 3, 4, and 2, while 1 and 5 were weakly or non-reactive against the construct. The results indicated, that both conjugates reacted remarkably similarity with antibodies except the serum 6 which contained antibodies preferring the structure including the immobilized cysteine as in natural peptides on viral surface.

### Peptide 2 as Aminoterminal Conjugate and Middle Cys-Conjugate

Biotin-aminohexanoic-SYACKR was tested against the 6 sera as N-terminal conjugate on a streptavidin plate. The sera 2 and 5 showed strongest immune response before sera 3,4 and 6, while serum 1 showed weakest reaction.

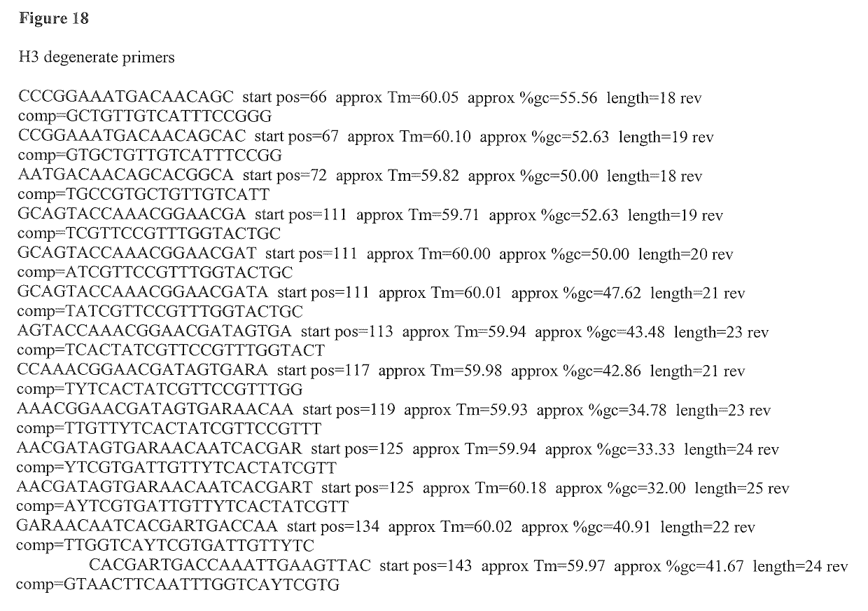

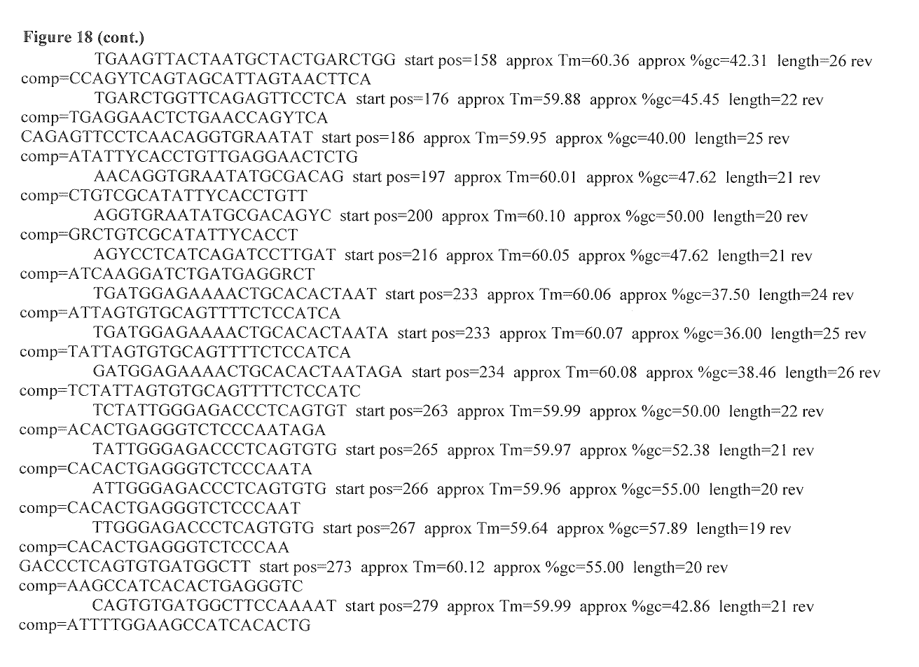

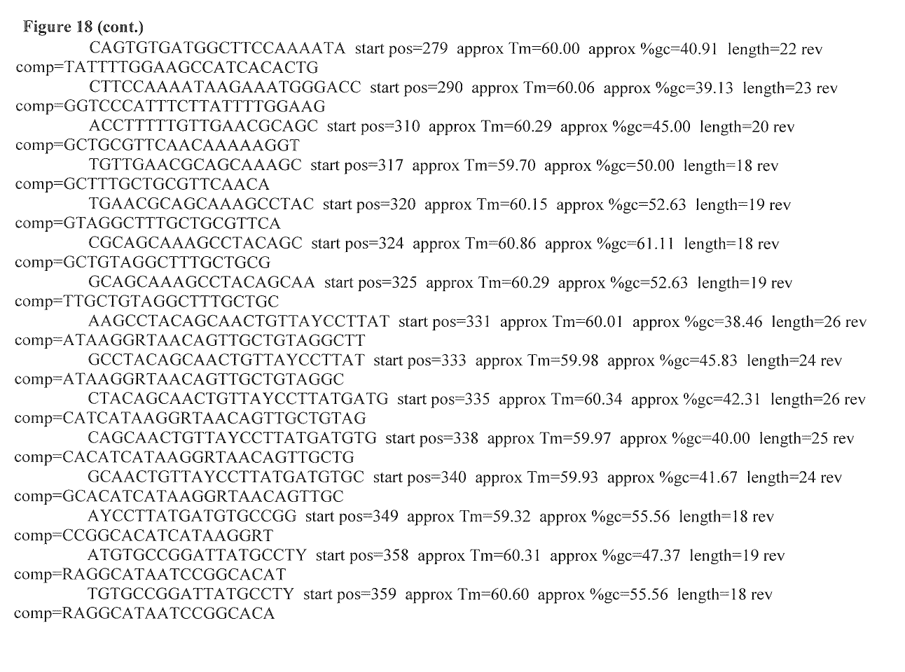

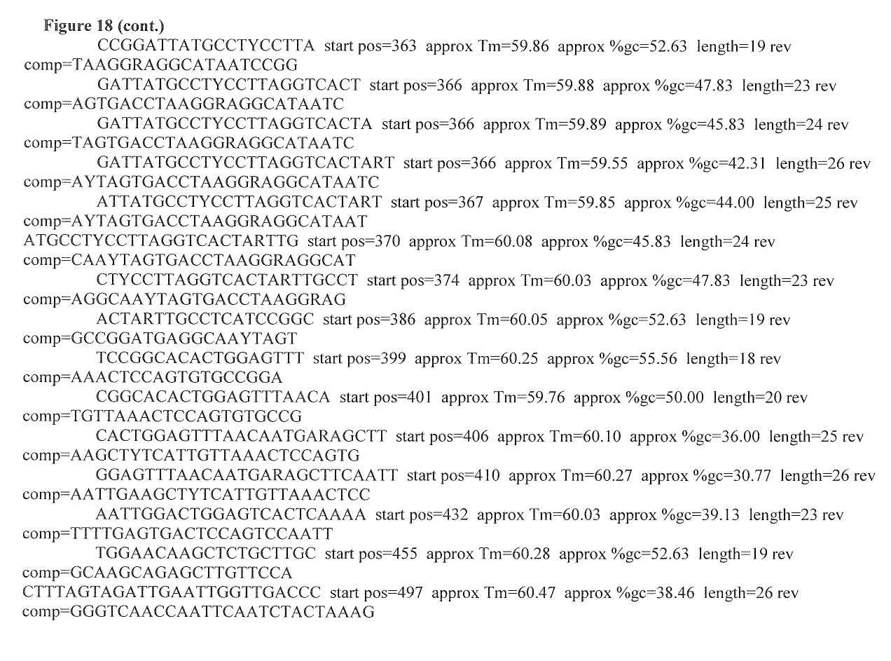

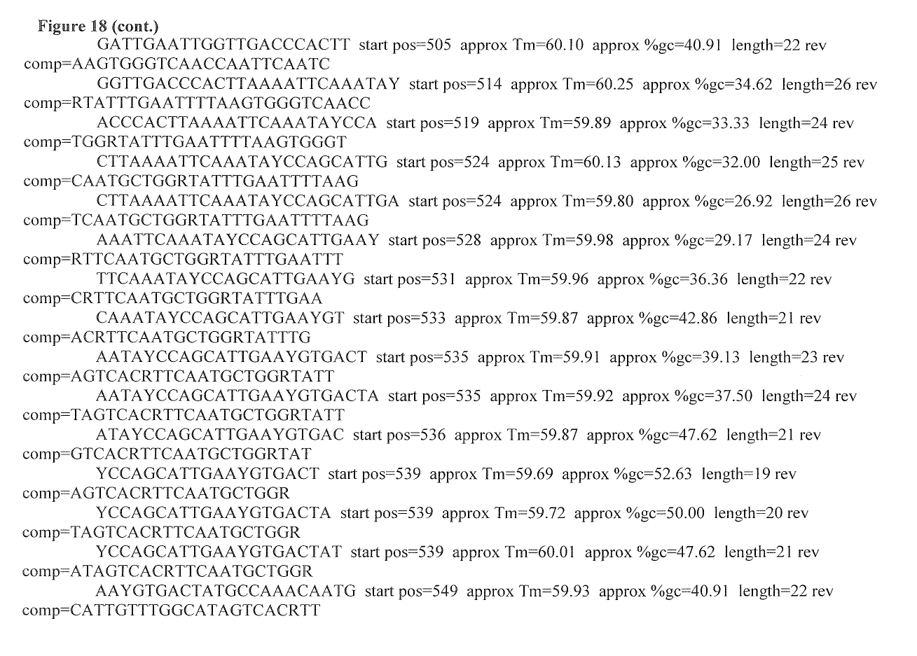

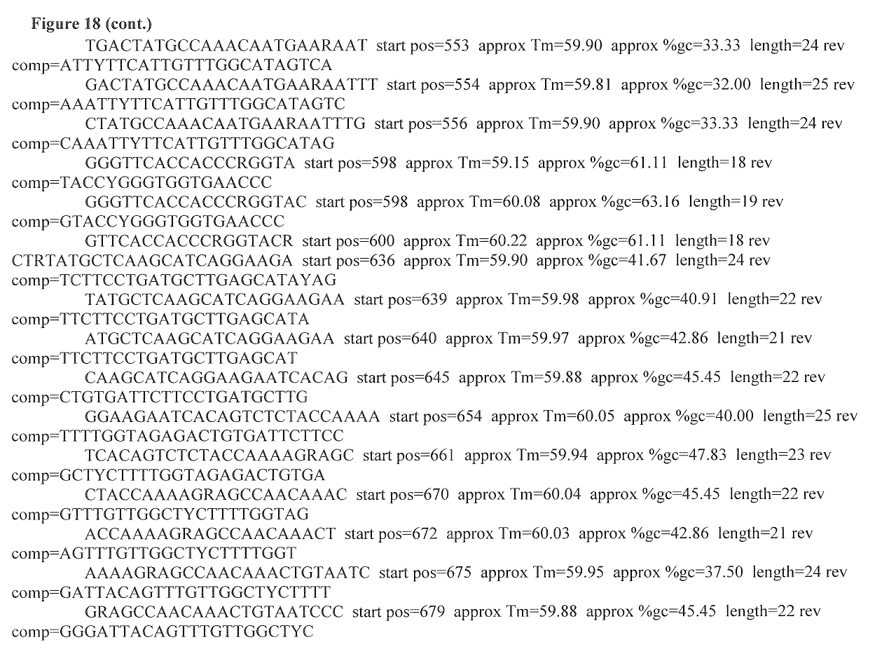

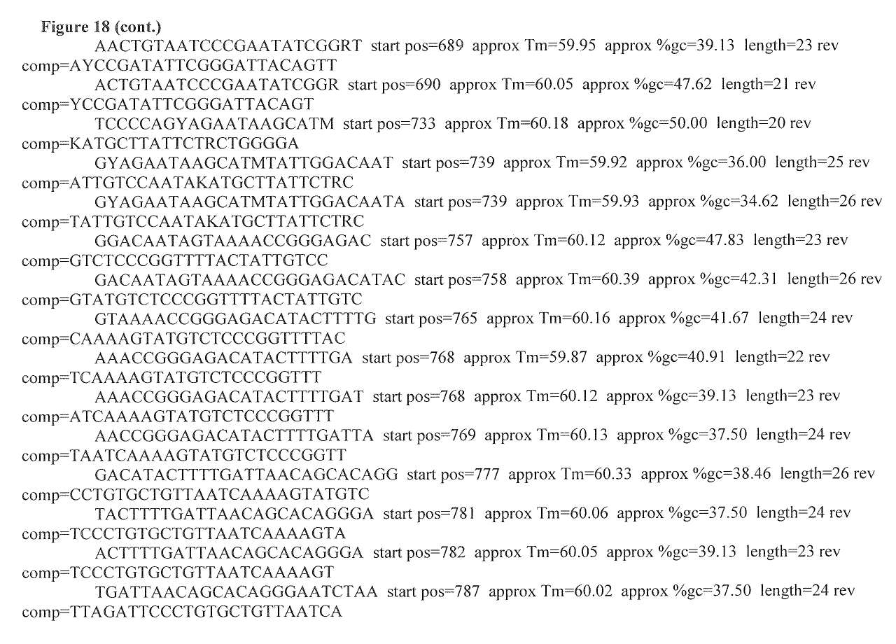

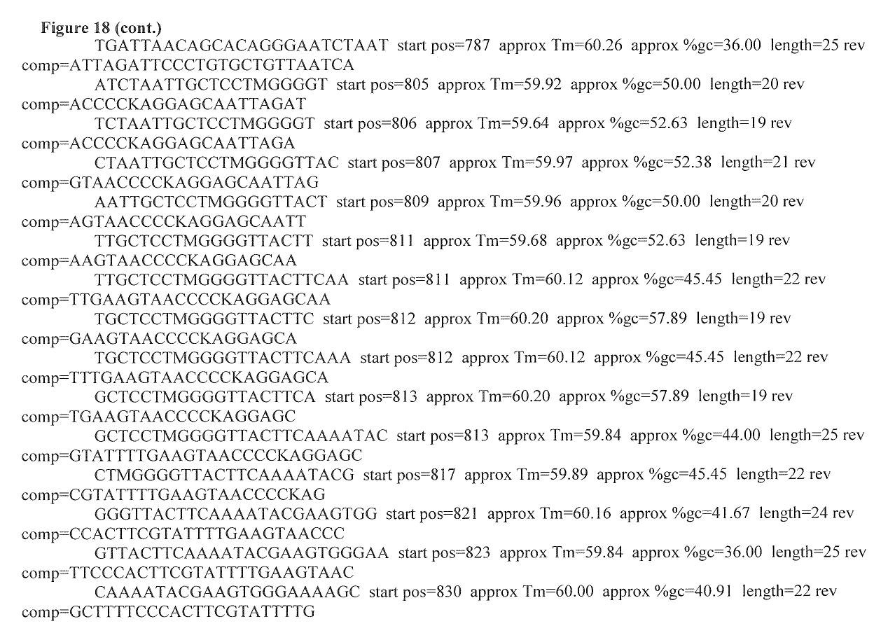

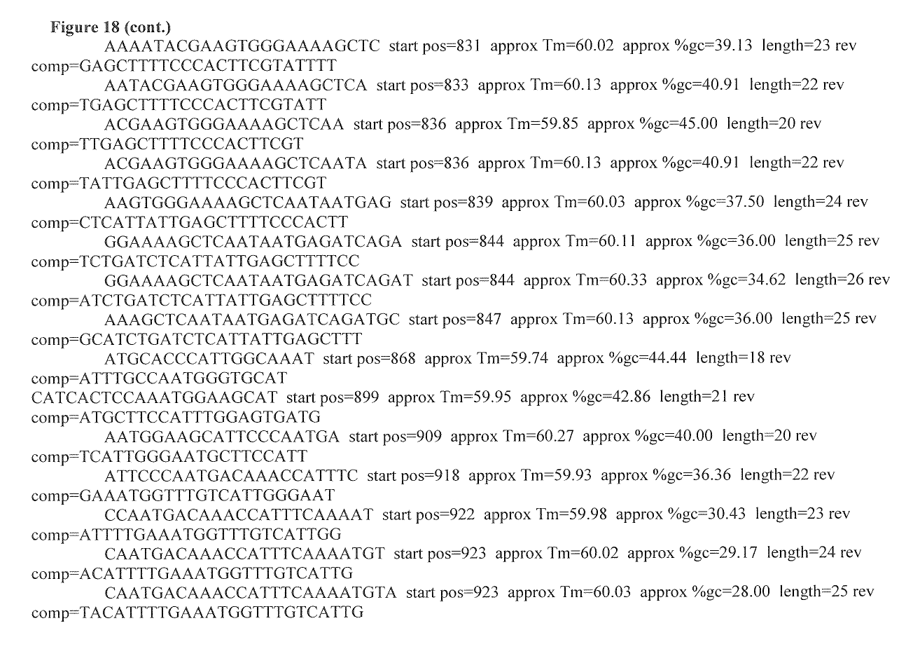

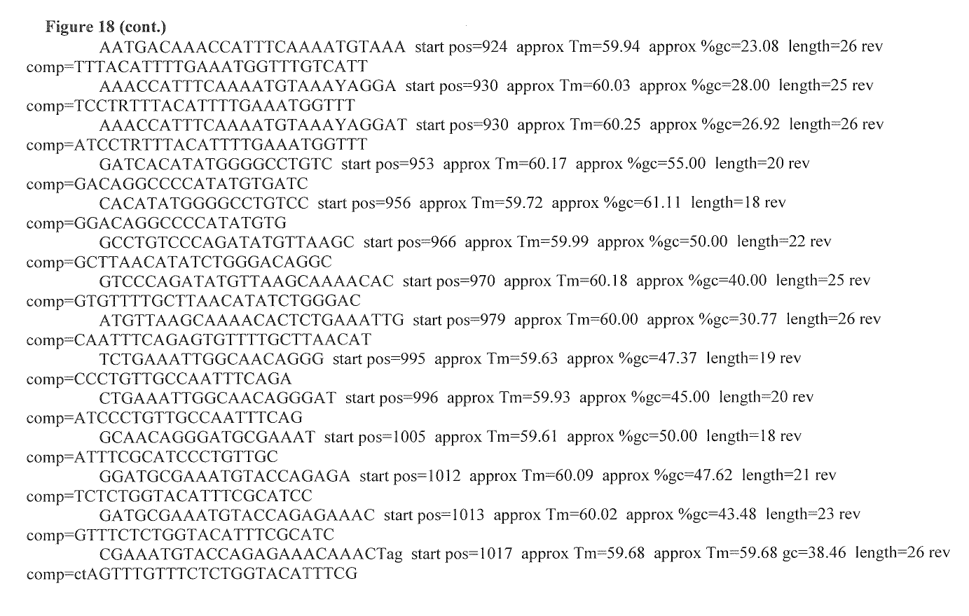

The middle cysteine conjugate of peptide 2 reacted with sera similarly but reactions with serum 5 was weaker and the serum 6 showed the strongest response, see FIG. 18 and Table 5. The results indicated, that both conjugates reacted remarkably similarly with antibodies except the serum 6 which contained antibodies preferring the structure including the immobilized cysteine as in natural peptides on viral surface.

### Peptide 3

Peptide 3 has distinct pattern of immune recognition as shown in Table 5.

Correlation of the Immune Reaction with Viral Presentation of the Peptides 1-3 and HA11

More than hundred recently cloned human influenza A viruses were studied with regard to presentation of peptides 1-3. It was realized that there is one to a few relatively common escape mutants of each one of these, which would be different in antigenicity in comparison to the peptides 1-3. The analysis further reveled that on average the viruses contain two of the peptides 1-3. Thus the result that each influenza resistant test subject had antiserum at least against two of peptides fits well data about the recent viruses in Finland. The data further support the invention about combination of the antigenic peptides. The combination of at least two peptides is preferred.

The control core sequence HA11 is present as very conserved sequence in most influenza A viruses and thus all persons would have been immunized against it as shown by the results in Table 5.

### Example 3

### Analysis of Conserved Peptide Epitopes 1-3 in Hemagglutinins H1, H2, and H3

The presence of hemagglutinin peptide epitopes 1-3 were analysed from hemagglutinin sequences. Tables 6 and 7 shows presence of Peptides 1-3 in H1 hemagglutinins as typical H1 Peptide 1-3 sequences. The analysis revealed further sequences, which are conserved well within H1 hemagglutinins These are named as PrePept1-4 and PostPept1-4. These conserved aminoacid sequences are preferred for sequence analysis and typing of influenza viruses. The PrePept1-3 and PostPept1-4 sequences were found to be characteristics for H1, with partial conservation of amino acid residue. The PrePept4 in its two forms WGVHHP and more rarely homologous WGIHHP were revealed to be very conserved among all A-influenza viruses.

Table 8 shows Peptide 1-3 sequences from selected H2 viruses. Characteristic sequences for H2-type influenza viruses were revealed.

Table 9 shows analysis Peptides 1-4 from large group recent human influenza viruses containing H3 hemagglutinins Several homologous sequences for each peptides 1-3 were revealed.

When comparing with data of serum Elisa experiment (see Example 2) a correlation was revealed. In most of the strains only one peptide epitope is likely mutated in the virus, which had immunized the persons, in comparison to peptides selected for the assay. As the immune defense had been likely obtained during 80′ and/or 90′ as the persons have not had severa influenza during recent years, the recent variants of peptide 1 and 2 were likely not causing the antibody production, which might have been yielded less pronounced reaction against the peptides 1-3 used in the ELISA experiment. The non-reactivity against peptide 1 may have been caused by X31 type SKAFSN-immunization during earlier decades when this type of sequence would have more frequent, but the antibodies would be less reactive with the hydrophilic variant of SKAYSN used in the experiments.

The invention is further directed to the use of the conserved PrePept and Post Pept sequences for analysis of corresponding Peptide 1-4 sequences. The conserved sequences may be used for example as targets of specific protease sequencing reagents of nucleic acid sequencing reagents such as RT-PCR primers. The peptide 1 can effectively sequences by using closely similar PrePept1 and PostPept1 sequences or other PostPept sequences (which would also yield other Peptide 2, 3 and/or 4 sequences depending on the selection of PostPeptide).

The invention is further directed to analysis of the carbohydrate binding status and/or infectivity of an influenza virus by analysing the sequence of Peptides 1-3 and/or Peptide 4. The invention is directed to the analysis by sequencing the protein and/or corresponding nucleic acids or by recognizing the peptides by specific antibodies, preferably by specific human antibodies.

TABLE 1
Summary of interactions between hemagglutinin
X31 Aichi and saccharide 7

Interactions

REGION A

| | |
|---|---|
| Conserved a.a.* | |
| Tyr98 | Hb between Tyr OH and Siaα6 O9 |
| Gly135 | Hydrophobic patch: Gly —CH₂ and |
| | Siaα6 acetamido —CH₃ |
| Ser136 | Hb between Ser OH and Siaα6 ⁻OOC— |
| Trp153 | Hydrophobic patch: Trp indole and Siaα6 |
| | acetamido —CH₃ |
| His183 | Hb between His NH and Siaα6 O9 |
| Leu194 | VDW packing |
| Gly225 | Hairpin loop |
| Semi- or non- | |
| conserved a.a.* | |
| Gly134 | VDW packing |
| Asn137 | Hb between Asn NH and Siaα6 ⁻OOC— (long) |
| Ala138 | Hydrophobic patch: Ala —CH₃ and |
| | Leu226 —CH₃ |
| Thr155 | Hydrophobic patch: Thr —CH₃ and |
| | Trp153 indole |
| Glu190 | Hb between Glu COO⁻ and Siaα6 OH9 |
| Leu226 | VDW packing (see also Ala138) |

REGION B

| | |
|---|---|
| Conserved a.a.* | |
| Ser95 | Hb between Ser OH and Asp68 ⁻COO— |
| Val223 | VDW packing |
| Arg224 | Hydrophobic patch: Arg —CH₂—CH₂— |
| | and hydrophobic side of GlcNAcβ6 |
| Gly225 | Hairpin loop |
| Trp222** | Hydrophobic patch: Trp indole and hydrophobic |
| | side of Manα4GlcNAc of glycan linked to Asn165 |
| Asn165-linked | Possible interactions with saccharide 7 (only |
| glycan | first three glycan sugars are visible by X-ray |
| Semi- or non- | |
| conserved a.a.* | |
| Phe 94 | VDW packing |
| Asn96 | Hb between Asn amido C═O and GlcNAcβ6 O3 |
| Asn137 | Hb between Asn amido C═O and GlcNAcβ3 O6 |
| | (short arm) |
| Ala138 | Hydrophobic patch: Ala —CH₃ and |
| | Leu226 —CH₃ |
| Lys140 | Hydrophobic and electrostatic interactions with Glcβ |
| Arg207 | Hb between Arg guanidino NH and |
| | GlcNAcβ3 O4, VDW packing |

REGION C

| | |
|---|---|
| Conserved a.a.* | |
| Thr65 | Hb between Thr OH and Siaα6 ⁻OOC— |
| Ser71 | Hb between Ser OH and Siaα6 4OH |
| Glu72 | Salt bridge with Arg208 |
| Ser95 | Hb between Ser OH and Asp68 ⁻OOC— |
| Gly98 | Protein fold |
| Pro99 | Protein fold |
| Tyr100 | Hb between tyr OH and Galβ O4 |
| Arg269 | VDW packing (binding site floor) |
| Semi- or non- | |
| conserved a.a.* | |
| Ser91 | None |
| Ala93 | VDW packing |
| Tyr105 | Hb between Tyr OH and Siaα6 ⁻OOC— |
| | and Galβ4 O4 |
| Arg208 | Bidentate hb between Arg guanidino NH |
| | and Siaα6 O7 |

*Concerved, semi- or nonconcerved amino acids refer to a comparison between X31 Aichi and the one hundred most homologous seguences but all cited amino acids refer to X31 Aichi
**It should be noted that strains A/2/Japan/305/57 and A/PR/8/34 are not included in the one hundred most homologous sequences and that their binding of saccharides 7, 17 and 18 are significantly different from the other tested strains. Notably, they both lack the N-linked glycan at Asn165 and Trp222 bordering region B and also reveal significant differences in region C.

TABLE 2
Glycosidic torsion angles of saccharide
7 in complex with X31 Aichi

| Linkage | Angles |
|---|---|
| A | 48, 179 |
| B | 39, 170 |
| C | 73, −12 |
| D | −61, −166, 172 |
| E | −170, 21 |
| F | 55, −12 |
| G | −162, 170, 45 |
| H | 86, −154, 31 |
| I | 40, −26 |

Saccharide 7 with linkage abbreviations:
Neu5Acα2-6[G]Galβ1-4[A]GlcNAcβ1-3[F](Neu5Acα2-6[D]Galβ1-4[I]GlcNAcβ1-3[F]Galβ1-4[B]GlcNAcβ1-6[H])Galβ1-4[C]Glc

TABLE 3
Example of library of branched poly-N-acetylalctosamines
Including simple monosialylated structures.

| | |
|---|---|
| 1 | Neu5Acα2-6Galβ1-4GlcNAcβ1-3(Galβ1-4[Fucα1- |
| | 3]GlcNAcβ1-6)Galβ1-4Glc |
| 2 | Neu5Acα2-6Galβ1-4GlcNAcβ1-3(Galβ1-4GlcNAcβ1-6)LNβ1- |
| | 3Galβ1-4Glc |
| 3 | Neu5Acα2-6[Galβ1-4GlcNAcβ1-3(Galβ1-4GlcNAcβ1-3Galβ1- |
| | 4GlcNAcβ1-6)Galβ1-4Glc] |
| 4 | Neu5Acα2-6Galβ1-4GlcNAcβ1-3Galβ1-4GlcNAcβ1-3 |
| | (Neu5Acα2-6Galβ1-4GlcNAcβ1-6)Galβ1-4Glc |
| 5 | Neu5Acα2-6Galβ1-4GlcNAcβ1-3(Neu5Acα2-3Galβ1- |
| | 4GlcNAcβ1-3Galβ1-4GlcNAcβ1-6)Galβ1-4Glc |
| 6 | Neu5Acα2-3Galβ1-4GlcNAcβ1-3Galβ1-4GlcNAcβ1- |
| | 3(Neu5Acα2-6Galβ1-4GlcNAcβ1-6)Galβ1-4Glc |
| 7 | Neu5Acα2-6Galβ1-4GlcNAcβ1-3Galβ1-4GlcNAcβ1- |
| | 3(Neu5Acα2-3Galβ1-4GlcNAcβ1-6)Galβ1-4Glc |
| 8 | Neu5Acα2-3Galβ1-4GlcNAcβ1-3Galβ1-4GlcNAcβ1- |
| | 3(Neu5Acα2-3Galβ1-4GlcNAcβ1-3Galβ1-4GlcNAcβ1- |
| | 6)Galβ1-4Glc |
| 9 | Neu5Acα2-6Galβ1-4GlcNAcβ1-3Galβ1-4GlcNAcβ1- |
| | 3(Neu5Acα2-6Galβ1-4GlcNAcβ1-3Galβ1-4GlcNAcβ1- |
| | 6)Galβ1-4Glc |
| 10 | Neu5Acα2-3Galβ1-4GlcNAcβ1-3(Neu5Acα2-6Galβ1- |
| | 4GlcNAcβ1-3Galβ1-4GlcNAcβ1-6)Galβ1-4Glc |
| 11 | Neu5Acα2-6Galβ1-4GlcNAcβ1-3(Galα1-3Galβ1-4GlcNAcβ1- |
| | 3Galβ1-4GlcNAcβ1-6)Galβ1-4Glc |
| 12 | Neu5Acα2-6Galβ1-4GlcNAcβ1-3(GlcNAcβ1-3Galβ1- |
| | 4GlcNAcβ1-3Galβ1-4GlcNAcβ1-6)Galβ1-4Glc |
| 13 | [Neu5Acα2-6Galβ1-4GlcNAcβ1-3Galβ1-4Glc]₂-DADA-oxime |
| 14 | [Neu5Acα2-3Galβ1-4Glc]₂-DADA-oxime |
| 15 | [Neu5Acα2-6Galβ1-4GlcNAc]₂-DADA-oxime |
| 16 | Neu5Acα2-6Galβ1-4GlcNAcβ1-3Galβ1-4Glc-(Neu5Acα2- |
| | 6Galβ1-4GlcNAc-)DADA-oxime |

### Example 4

### Multiple Alignment of Amino Acid Sequences from Various HA Subtypes and Hosts

Altogether 158 sequences and 788 sequences were used for the analysis. In some cases all peptide sequences of a subtype were aligned in groups of 200-400 sequences. The sequences were aligned using Influenza Virus Resource alignment tools and the variant amino acids were visually observed within the peptide regions of the invention. Comparisons were also made within an HA subtype by aligning each HA subtypes and observing variation in the peptide regions of the invention.

### Example 5

### Designing of Primer Sequences

The representative selection of amino acid sequences of H1, H3 and H5 were aligned using Influenza Virus Resource net site or ClustalW. The consensus sequences were initially identified visually and they were further used for designing of primers for nucleotide analysis.

The primers were designed for consensus sequences upstream and downstream from the large binding site or peptides of the present invention. Some primers encompass one or two or three peptide regions. Some primers were directed to peptide4 which is hyper conserved among all HA studied.

Designing degenerate primers for H1 and H3 were taken separately because of deletions and insertions in the nucleotide sequences. However, areas devoid of deletions and insertions are suitable for degenerate primer analysis and preferred regions of the primer design for HA subtypes.

H1 sequences used for the degenerate primer design were the following: CY016394, CY013581, DQ265706, AY299503, DQ249260, AJ489852, AB255398 and CY016699.

H3 sequences used for the degenerate primer design were the following: DQ174268, DQ415324, DQ865951, DQ167304, AB259112, DQ114535, CY016995 and DQ865969.

H5 sequences used for the degenerate primer design were the following:

CY014529, AY555153, AB212054, DQ643809, DQ497729, and CY014197.

Degenerate PCR primers were designed using Kellogg degenerate primer software. Table 1-3 represent the location of primers and corresponding nucleotide start sites. The primers were designed so that they would anneal as many hemagglutinin subtypes as possible, preferably all hemagglutinin subtypes and most preferably at least H1 and H3. These degenerate primers without variation or with 1-2 degenerate nucleotides are shown in Tables 1-3. Complete list of all predicted primers are shown in FIGS. 17-19. Other primers identifying specific HA subtypes can also be designed and combined with each other.

The preferred primers for consensus sequences of HA comprise the following:

### Example 6

### Isolation of RNA and Detection of Influenza Virus Using Gel-Based Detection Platform

Experiments are performed on RNA extracted, for example, from eggs and from human clinical samples including allantoic fluid, cloacal and trachael swabs, homogenized tissue, pooled organs, blood, sputum, stools, urine and nasopharyngeal aspirates.

The following is a general protocol for detection of influenza virus subtypes H1-H5 or H6-H16.

Generally, RNA is extracted from samples according to the manufacturer's instructions, using either TRIzol™ or RNA extraction kits (Qiagen).

The first-strand cDNA synthesis is performed on extracted RNA using the relevant reverse primer(s) (2 μl of 10 μM stock) in a 20 μl reaction volume. A first round PCR reaction is set up using 2.5 μl of the cDNA reaction, containing cDNA product as template with relevant forward and reverse primer(s) (1.25 μl total volume for each of forward and reverse) in a 25 μl reaction volume. The PCR conditions are set up as follows: incubation at 94° C. for 2 min; 35 cycles of 94° C. for 10 sec, 50° C. for 30 sec, 72° C. for 1 min; followed by an incubation at 72° C. for 7 min. A second round of PCR is performed using the product of the first round PCR (2.5 μl) as template. All other conditions and reagents are the same as for the first round PCR.

The products of the second round PCR are analysed on a 1.5 to 2% agarose gel by staining with ethidium bromide.

However, in some cases that one-round of PCR will be sufficient for detection

The above RT-PCR protocol can be performed using RNA extracted from an HA viral isolates derived from various countries and samples.

### Example 7

### Detection of Influenza Virus HA Using Real-Time RT-PCR Detection Platform

The following reactions are performed in a LightCycler™ instrument.

The reaction master mixture is prepared on ice by mixing the following reagents in order, to a volume of 20 μl: water (volume adjusted as necessary), 50 mM manganese acetate (1.3 μl), ProbeNPrimer mix containing forward primer and reverse primer to a final concentration of 0.2 to 1 μM and fluorescently labelled probes (2.6 μl), LightCycler RNA Master Hybridization Probes (7.5 μl), which contains buffer, nucleotides and enzyme.

The reactions are transferred to glass capillary tubes suitable for use in the LightCycler™ 5 μl of extracted RNA template is added to each reaction and briefly centrifuged. The RT-PCR reactions are run using the well established programs which are suited for the present invention. For example, the 8 primer sets can be designed and reactions are performed using SYBR green fluorescent detection kit, in accordance with standard protocols and commercially available reagent kits (Roche).

The sensitivity of the primers using the real time PCR protocol can be assessed from amplification curves generated to monitor the production of amplification product. Generally, specific amplification products will have a higher melting temperature than non-specific products, and the melting curve profile can be used to confirm the specificity of the reaction.

### Example 8

### DNA Microarray Using Primers

Particular primers of the invention can be used in a DNA micro array (Attogenix, Singapore) to detect RNA from HA isolates. Briefly, various HA primers are immobilized on a solid surface (GAPDH can be used as a positive control for RT-PCR). The micro array is then probed with sample HA transcript. RNA binding of the probe to the primer in each spot in the micro array is detected using SYBR Green fluorescent probe to detect double-stranded nucleic acid.

### Example 9

### Determination of Protein Epitopes in a Patient and Administration of Peptide Antigens

The protein epitopes of an influenza virus are determined as described above. A sample is taken from an infected patient, or animal, or from any place or specimen which is suspected to contain HA. Primers of present invention are used to determine the protein epitope composition of the HA. Thereafter, peptide epitopes are administered into a patient so that immune response occurs, or patients are vaccinated using peptide epitopes formulated in suitable pharmaceutical composition.

### Example 10

### Analysis of Current Influenza Peptides Including Cyclic Forms of Peptides 3

Linear and cyclic peptides from recent influenza H1 and H3 viruses were tested for binding to antibodies from serum of 8 persons similarly as in ELISA assay as in Example 2. The process was optimized increasing washing the plates. The assay revealed strong immune individual specific responses against all tested peptides. This is partially expected to be based on the infections of person by older viruses or more current H1 and/or H3 viruses with current sequences

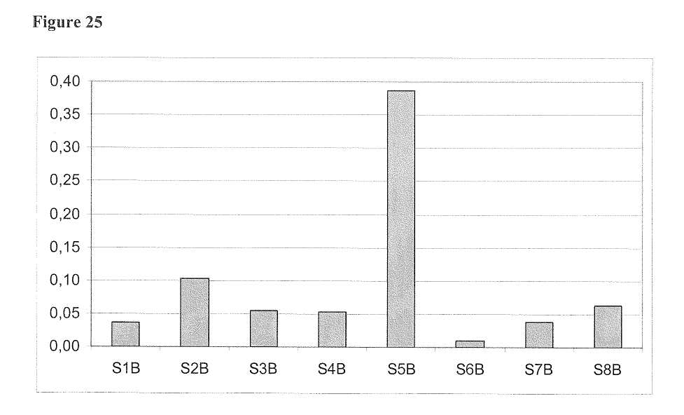

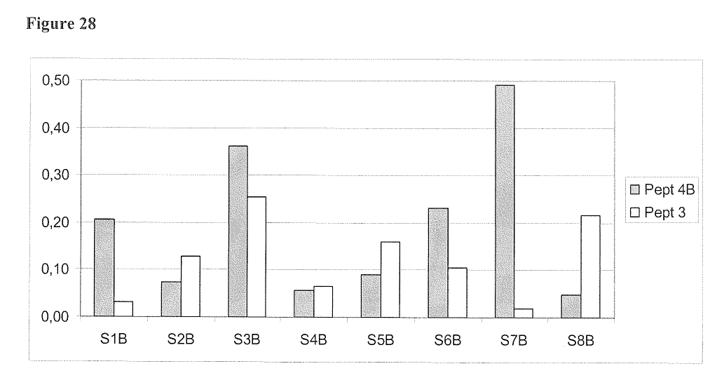

The assays revealed especially that cyclic peptides 3 in cyclic form are especially strong immugens/antibody targets. FIG. 28 shows that cyclic Peptide 4b bind generally more strongly antibodies than the corresponding linear peptide 3 analyzed again (also used in Example 2). Also the H1 peptide 3 in cyclic form showed unusually high response, especially with a person S5B, FIG. 25, who had been vaccinated against influenza (vaccines comprise regularly both H1 and H3 virus though the infection with H1 may be otherwise more rare). This indicates that the binding of conformational structure 3 is especially useful. It is realized that in Example 2 the differences in maleimide linked epitope linking conformationally from cysteine and the N-terminally linked structures from biotin indicates that the cystein linkage would provide beneficial conformational peptide for certain natural anti-influenza antibodies.

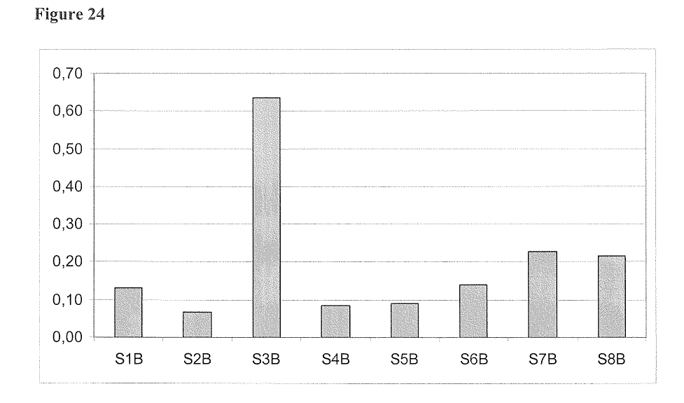

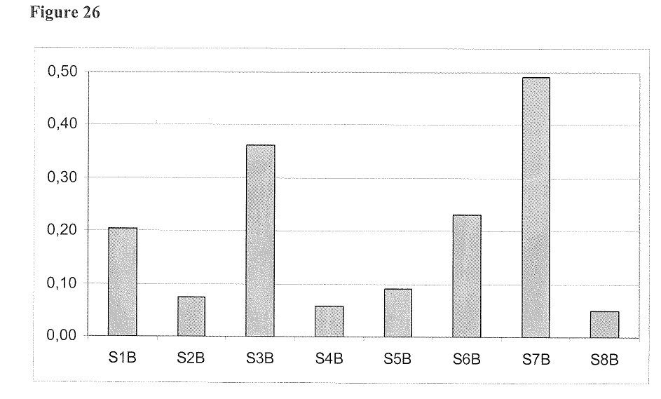

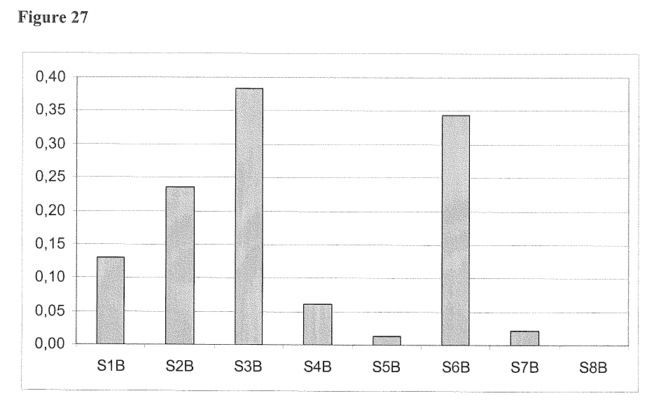

It is thus realized that the novel peptides are useful in recognition of influenza immunoreactions in context of vaccination with whole viruses or larger hemagglutinin peptides or proteins, person S5B FIG. 25. It is further realized and preferred that immunoassays directed to measuring the antibodies against influenza are especially useful for diagnosis of influenza and even specific type of influenza with regard to hemagglutinin structures. At least persons S3B (required hospital visit) and S7B were considered as recently infected quite severely with influenza and showed strong immune responses to new peptides as shown in FIGS. 24 and 26, (may be partially 23). The immune responses to older cyclic peptide of FIG. 28, for S3B was considered to originated from earlier infection likely with old H3 virus.

It is further realized that the cyclic peptide 3 from H1 RPKVRDQ, FIG. 25, and corresponding sequences of current H3 RPRVRNI, and even to certain level older H3 sequence (now infecting more animals especially pigs) RPWVRGL, tested are substantially homologous with avian influenza H5 peptide 3 with sequence RPKVNGQ. It is thus realized that the peptides have tendency for conservation, especially H1 peptides are preferred because of conservation from spanich flu ((A(South Caroline/1/18). The invention is in a preferred embodiment directed to use of the preferred peptides 2 and 3, more preferably

The novel H1 and H3 peptides 2 and 3 showed strong immune reactions especially in person who had been indicated to have been infected recently with influenza. The invention also revealed that linear peptide 3 of current H3 influenza comprising a conformational additional amino acid residue(s) including proline at the carboxyl terminus was especially effective in binding with certain antibodies.

### Experimental Process

### Materials and Equipments

### Plates:

Reacti-Bind Streptavidin Coated Clear Strip Plates with Blocker BSA, Pierce, prod. no 15121

### Reagents:

PBS, Phosphate Buffered Saline, 10 mM Na-phosphate buffer, 0.15 M NaCl, pH 7.2

Washing buffer: 0.2% BSA in PBS with 0.05% Tween-20.

BSA, Bovine Serum Albumin

### Equipments:

Certomat R M, B. Braun Biotech International

Multiscan Spectrum (re w cuvette), Thermo Electron

### Procedure:

Blocking: Incubation with 150 μl of 0.5% BSA in PBS with 0.05% Tween-20 for 1 h at room temperature (RT) with shaking (75 rpm, Certomat).

Washing: Three times with 200 μl of PBS with 0.05% Tween-20 with shaking for three minutes (150 rpm, Certomat).

Antigen binding: Incubation with 100 pmol of biotinylated peptide in 100 μl PBS for 0.5 h at RT with shaking (75 rpm, Certomat) and then overnight at +4° C.

Washing: Each well five times with 200 μl of Washing buffer, incubation each time for three minutes with shaking (150 rpm, Certomat).

Primary antibody: Serum from eight individuals were used as primary antibody dilutions, the serial dilutions (in Washing buffer) were: 1:10, 1:100, and 1:1000.

Incubation with 100 μl of diluted serum for 1 h at RT with shaking (75 rpm, Certomat).

Washing: Ten times with 200 μl of Washing buffer, incubation each time for three minutes with shaking (150 rpm, Certomat).

Enzyme labeled secondary antibody: As secondary antibody 1:30000 dilution of Anti-Human Polyvalent Immunoglobulins (G, A, M) Peroxidase conjugate (Sigma) was used. Incubation with 100 μl of diluted immunoglobulins reagent for 1 h at RT with shaking (75 rpm, Certomat). Washing: Eight times with 200 μl of Washing buffer, incubation each time for three minutes with shaking (150 rpm, Certomat).

Determining binding activity: Incubation with 100 μl of TMB+ Substrate Chromogen (S5199, DacoCytomation, CA, USA) for 15 minutes at RT with shaking (75 rpm, Certomat).

Ending the enzymatic reaction by 100 μl 1 M H₂SO₄, shaking (75 rpm, Certomat) for three minutes. Measuring the absorbance at 450 nm.

Serum dilutions without antigen (=biotinylated peptide) were measured for unspecific binding (i.e. control samples).

### Peptides 1B-5B

(Aminocaproyl=aminohexanoyl, biotin at N-terminus)
H=hemagglutinin

### Peptide 1B

### Biotin-aminocaproyl-GTSSACIRR

Represents the peptide 2 from current H3 variant

### Peptide 2B

### Biotin-aminocaproyl-SRPRVRNIP

Represents the peptide 3 from current H3 variant

### Peptide 3B

Biotin-aminocaproyl-CRPKVRDQC, cyclic peptide having disulfide bridge from Cys to Cys
Represents the peptide 3 from former H1 variant

### Peptide 4B

Biotin-aminocaproyl-CRPWVRGVC, cyclic peptide having disulfide bridge from Cys to Cys
Represents the peptide 2 from former H3 variant; similar to Peptide 3 except that this is cyclic

### Peptide 5B

### Biotin-aminocaproyl-GVSASCSH

Represents the peptide 2 from H1 variant

### Serum Indications

### Serum 1B (SIB)

Individual indicates that according to symptoms he/she most probably had influenza on spring 2007. Serum of this individual was studied on ELISA experiments performed 2006, serum number was S2 (in Example 2).

### Serum 2B (S2B)

No indication of influenza. Serum of this individual was studied on ELISA experiments performed 2006, serum number was S5.

### Serum 3B (S3B)

Diagnosis made by medical doctor indicates that individual had influenza on spring 2007. Symptoms were so severe that he/she was hospitalized for one day. Has had also influenza on 1999.

### Serum 4B (S4B)

No indication of influenza. Serum of this individual was studied on ELISA experiments performed 2006, serum number was S6.

### Serum 5B (S5B)

Individual has been vaccinated against influenza on Winter 2002-2003 at USA.

### Serum 6B (S6B)

No indication of influenza. Serum of this individual was studied on ELISA experiments performed 2006, serum number was S4.

### Serum 7B (S7B)

Individual indicates that he/she had influenza on spring 1997. Serum of this individual was studied on ELISA experiments performed 2006, serum number was S3.

### Serum 8B (S8B)

No indication of influenza for this individual.

### Example 11

Blast (enterez web site) searches were performed with amino acid sequences Peptides 1-3. Similarity in human genome sequences were found especially for peptide 1 of H1 and H3. Relevance of the similarity is analyzed by estimating presence of the structures on cell surface proteins and on proteins surfaces when/if 3D structures are available. Three dimensional structures on patients (human or animal) peptides are considered.

### Example 12

Polyvalent conjugates of Peptide 1, Peptide 2 and Peptide 3 spacer modified (amihenoyl spacer) KLH protein are produced. Mice are immunized with conjugates and specific immune responses are observed. The example indicates suitability of the peptides for animal immunization. Similar experiments are performed with preferred animal patients: pigs and chicken to which the human viruses are more relevant and with horses. The human antibody data indicates as retrospective clinical trial usefulness for specific treatment of human. It is realized that immunization can be performed in multiple was cited in the references of the application.

TABLE 5
Approximate immune reactions of sera from
test subjects 1-6 against synthetic peptides.

| | P1-N | P1-C Cys | P2-N | P2-mid Cys | P3-N | HA11-N Cys |
|---|---|---|---|---|---|---|
| Serum 1 | − | − | ++ | + | ++ | + |
| Serum 2 | + | + | ++++ | +++ | + | +++ |
| Serum 3 | ++ | ++ | ++ | +++ | − | ++ |
| Serum 4 | ++ | ++ | +++ | + | − | ++ |
| Serum 5 | − | − | +++ | + | + | + |
| Serum 6 | − | +++ | +++ | +++ | + | ++ |

P1, P2, And P3 indicates peptides 1-3, HA11 is commercial peptide N is N-terminal Biotin immobilized conjugate, Cys-indicates Cys-conjugate, C is C-terminal.

TABLE 6

Conserved/antigenic peptide epitopes including Peptides 1-3 in selected
H1-hemagglutinins. Prev indicates previous aminoacid belonging as additional
residues in the epitope, pos indicates the position of the aminoacid residue
in the hemagglutinin sequence, Past indicates foolowing amino acid residue
belonging as additional residues in the epitope. Sequence indicates not so
frequent variants of the sequence. PrePept and Post Pept sequences are
additional conserved and/or antigenic sequences in the peptide. In column 2,
s indicates presence of possible signal peptide affecting the numbering, s13
is putative signal peptide of 13 amino acid residue, when sequence positions
are compared the signal peptides may be deducted from the aminoacid
position numbers.

| | | | | Peptide1 | | | | | | | |
|---|---|---|---|---|---|---|---|---|---|---|---|
| | | | | NSENGTC(a) | | | | PostPept1 | | | |
| | | PrePept1 | | NPENGTC(b) | | | | YPGDFIDYE(a) | | | |
| Hemagglutinin type H1 | | SWSYI(a) | | NSENGIC(c) | | | | YPGYFADYE(b) | | | |
| Virus name | | prev.Pos.past | a) | prev.Pos. | a) | b | c) | c) sequence | a) | b) | |
| A/South Carolina/1/18 | H1N1 | AS79-83VE | 1 | TS88-94 | 1 | | | | 1 | | 95-103 |
| A/Finland/158/91 | H1N1, s13 | KE92-96AE | 1 | TP101-107 | | 1 | | | | 1 | 108-116 |
| A/Mongolia/111/91 | H1N1, s | VR88-92VE | 1 | TP97-103 | | | 1 | | 1 | | 104-112 |
| A/Czechoslovakia/2/88 | H1N1, s13? | KK92-96AE | 1 | TP101-107 | 1 | | | | | 1 | 108-116 |
| A/Fiji/2/88 | H1N1, s13 | KK92-96AE | 1 | TP101-107 | 1 | | | | | 1 | 108-116 |
| A/Trinidad/2/86 | H1N1, s13 | KK92-96AE | 1 | TP101-107 | 1 | | | | | 1 | 108-116 |
| A/duck/WI/259/80 | H1N1, s13 | AN92-96IE | 1 | TS101-107 | 1 | | | YPGEFIDYE | | | 108-116 |
| A/Mongolia/231/85 | H1N1, s13 | KK92-96AE | 1 | TP101-107 | 1 | | | | | 1 | 108-116 |
| A/Texas/22/90 | H1N1, s13 | KE92-96AE | 1 | TP101-107 | | 1 | | | | 1 | 108-116 |

| | | | | | | | Peptide2b | | | |
|---|---|---|---|---|---|---|---|---|---|---|
| | | | | Peptide2 | | | SYAGAS(a) | | | |
| | PrePep2 | | | GVTAAC(a) | | | SHNGKS(b) | | | |
| Hemagglutinin type H1 | SSWPNH(a) | | | GVTASC(b) | | | SHEGKS(c) | | | |
| Virus name | b)sequen. | a) | Prev.Pos.Past | Prev.Pos | a) | b) | d)sequence | a) | b) | c) |
| A/South Carolina/1/18 | | 1 | KT125-130HE | TK135-140 | 1 | | | 1 | | |
| A/Finland/158/91 | | 1 | KE138-143TV | TK148-153 | | 1 | | | 1 | |
| A/Mongolia/111/91 | | 1 | KE134-199N | TN143-148 | 1 | | | | | 1 |
| A/Czechoslovakia/2/88 | | 1 | KE138-143TV | TK148-153 | 1 | | SHKGRS | | | |
| A/Fiji/2/88 | | 1 | KE138-143TV | TK148-153 | | 1 | SHKGKS | | | |
| A/Trinidad/2/86 | | 1 | KE138-143TV | TK148-153 | | 1 | SHKGKC | | | |
| A/duck/WI/259/80 | | 1 | KA138-143ET | TK148-153 | 1 | | SYSGAS | | | |
| A/Mongolia/231/85 | RSWPKH | | KE138-143NV | TR148-153 | | 1 | SHKGKS | | | |
| A/Texas/22/90 | | 1 | KE138-143TV | TK148-153 | | 1 | | | 1 | |

TABLE 7

Conserved/antigenic peptide epitopes including Peptides 1-3 in selected
H1-hemagglutinins. The abbreviation are as in Table 6.

| | | | | | Peptide4 | | | | |
|---|---|---|---|---|---|---|---|---|---|
| | | PrePept4 | | | TDQQSLYQ(a) | | | | |
| | | WGVHHP(a) | | | GDQRAIYH(b) | | | | |
| Hemagglutinin type H1 | | WGIHHP(b) | | | KEQQNLYQ(c) | | | | |
| Virus name | | Prev.Pos.Past | a) | b) | d)sequence | a) | b) | c) | Pre.Pos.Past |
| A/South Carolina/1/18 | H1N1 | VL181-186 PT | 1 | | | 1 | | | G190-197NADAYVSVG |
| A/Finland/158/91 | H1N1, s13 | VL194-199 SN | 1 | | | | 1 | | I203-210TENAYVSVV |
| A/Mongolia/111/91 | H1N1, s | VL189-194 PN | | 1 | | | | 1 | S198-205NENAYVSVV |
| A/Czechoslovakia/2/88 | H1N1, s13? | VL194-199 SN | 1 | | | | 1 | | I203-210TENAYVSVV |
| A/Fiji/2/88 | H1N1, s13 | VL194-199 SN | 1 | | GNQRAIYH | | | | I203-210TENAYVSVV |
| A/Trinidad/2/86 | H1N1, s13 | VL194-199 SN | 1 | | | | 1 | | I203-210TENAYVSVV |
| A/duck/WI/259/80 | H1N1, s13 | VL194-199 PT | 1 | | NEQQSLYQ | | | | V203-210NADAYVSVG |
| A/Mongolia/231/85 | H1N1, s13 | VL194-199 SN | 1 | | EDQKTIYR | | | | I203-210 KENAYVSVV |
| A/Texas/22/90 | H1N1, s13 | VL194-199 SN | 1 | | RDQRAIYH | | | | I203-210TENAYVSVV |

| | | | | | | PostPeptide3 | | | |
|---|---|---|---|---|---|---|---|---|---|
| | PrePept3/ | | Peptide3 | | | NYYWTLL(a) | | | |
| | PostPept4 | | RPKVRDQ(a) | | | NYYWTML(b) | | | |
| Hemagglutinin type H1 | RRFTPEI | | RPKVRGQ(b) | | | NYHWTLL(c) | | | |
| Virus name | Prev.PosPast | a) | Prev.PosPast | a) | b) | Pre.PosPast | a) | b) | c) |
| A/South Carolina/1/18 | KYN212-218 | 1 | AA221-227A | 1 | | GRM232-238EPGDTI | 1 | | |
| A/Finland/158/91 | HYS225-231 | 1 | AK234-240E | 1 | | GRI245-251EPGDTI | 1 | | |
| A/Mongolia/111/91 | NYN220-226 | 1 | AE229-235A | 1 | | GRM240-246KPGDTI | 1 | | |
| A/Czechoslovakia/2/88 | HYN225-231 | 1 | AK234-240E | 1 | | GRI245-251EPGDTI | 1 | | |
| A/Fiji/2/88 | HYN225-231 | 1 | AK234-240E | | 1 | GRI245-251EPGDTI | | 1 | |
| A/Trinidad/2/86 | HYN225-231 | 1 | AK234-240E | 1 | | GRI245-251EPGDTI | 1 | | |
| A/duck/WI/259/80 | KYN225-231 | 1 | AA234-240A | | 1 | GRM245-251DQGDTI | | | 1 |
| A/Mongolia/231/85 | NYN225-231 | 1 | AE234-240G | | 1 | GRI245-251EPGDTI | 1 | | |
| A/Texas/22/90 | HYS225-231 | 1 | AK234-240E | 1 | | GRI245-251EPGDTI | 1 | | |

TABLE 8

Conserved/antigenic peptide epitopes, Peptides 1-3, in selected
H2-hemagglutinins. The abbreviation are as in Table 6.

| | | | | | | | | | Peptide3 | | |
|---|---|---|---|---|---|---|---|---|---|---|---|
| | | Peptide1 | | | | Peptide2 | | | RPEVNGQ(a) | | |
| | | NPRNGLC(a) | | | | SQGCAV(a) | | | RPKVNGL(b) | | |
| | | NPRYSLC(b) | | | | SWACAV(b) | | | (c) | | |
| | | (c) | | | | (c) | | | (d) | | |
| Virus name | H2 | Prev.PosPast | a) | b | | Prev.PosPast | a) | b) | Prev.PosPast | a) | b) |
| A/chick./Potsdam/4705 | H2N2, s | KE99-105 | 1 | | KE99-105 | TTGG146-15 | 1 | | AT230-236GG | 1 | |
| A/Korea/426/68 | H2N2, s | KE99-105 | | 1 | KE99-105 | TTGG146-151S [symbol] | | 1 | AA230-236GR | | 1 |

[symbol]

indicates data missing or illegible when filed

TABLE 9
Conserved/antigenic peptide epitopes including Peptides 1-3 in selected H3-hemagglutinins.

| | Peptide1 | PostPept1 | Peptide2 | Post Pept2 | PrePept4 |
|---|---|---|---|---|---|
| Hemagglutinin type H3 | SKAFSNC(a) | YPYDVPDYA(a) | SNACKR(a) | GFFSRL(a) | WGVHHP(a) |
| | SKAYSNC(b) | YPYDVPDYV(b) | SYACKR(b) | SFFSRL(b) | WGIHHP(b) |
| | STAYSNC(c) | | SSACKR(c) | | |

| Virus name | | d) sequen. | a) | b | c) | Prev. Pos. | Pos. | a) | b) | Prev. PosPast | a) | b) |
|---|---|---|---|---|---|---|---|---|---|---|---|---|
| A/X31 | H3N2 | | 1 | | | ER91-97 | 98-106 | 1 | | QNGG136-141GPGS | 1 | |
| A/Finland/445/96 | H3N2 | | | | 1 | 91-97 | 98-106 | 1 | | 136-141 | | 1 |
| A/Finland/539/97 | H3N2 | STAYSNC | | | | 91-97 | 98-106 | 1 | | 136-141 | | 1 |
| A/Finland/447/96 | H3N2 | SKAYSDC | | | | 91-97 | 98-106 | 1 | | 136-141 | | 1 |
| A/Finland/313/03 | H3N2 | SKADSNC | | | | 91-97 | 98-106 | 1 | | 136-141 | | |
| A/Finland/594/98 | H3N2 | | | 1 | | 91-97 | 98-106 | 1 | | 136-141 | | 1 |
| A/Finland/587/98 | H3N2 | | | 1 | | 91-97 | 98-106 | 1 | | 136-141 | | 1 |
| A/Finland/590/98 | H3N2 | | | 1 | | 91-97 | 98-106 | 1 | | 136-141 | | 1 |
| A/Finland/528/97 | H3N2 | | | 1 | | 91-97 | 98-106 | 1 | | 136-141 | | 1 |
| A/Finland/339/95 | H3N2 | | | 1 | | 91-97 | 98-106 | 1 | | 136-141 | | 1 |
| A/Finland/380/95 | H3N2 | | | 1 | | 91-97 | 98-106 | 1 | | 136-141 | | 1 |
| A/Finland/364/95 | H3N2 | | | 1 | | 91-97 | 98-106 | 1 | | 136-141 | | 1 |
| A/Finland/296/93 | H3N2 | | | 1 | | 91-97 | 98-106 | 1 | | 136-141 | | 1 |
| A/Finland/256/93 | H3N2 | | | 1 | | 91-97 | 98-106 | 1 | | 136-141 | | 1 |
| A/Finland/321/93 | H3N2 | | | 1 | | 91-97 | 98-106 | 1 | | 136-141 | | 1 |
| A/Finland/263/93 | H3N2 | | | 1 | | 91-97 | 98-106 | 1 | | 136-141 | | 1 |
| A/Finland/190/92 | H3N2 | | | 1 | | 91-97 | 98-106 | 1 | | 136-141 | | 1 |
| A/Finland/218/92 | H3N2 | | | 1 | | 91-97 | 98-106 | 1 | | 136-141 | | 1 |
| A/Finland/191/92 | H3N2 | | | 1 | | 91-97 | 98-106 | 1 | | 136-141 | | 1 |
| A/Finland/110/89 | H3N2 | | | 1 | | 91-97 | 98-106 | 1 | | 136-141 | | 1 |
| A/Finland/220/92 | H3N2, s16 | | | 1 | | 107-113 | 114-122 | 1 | | 152-167 | | 1 |
| A/Finland/218/92 | H3N2, s16 | | | 1 | | 107-113 | 114-122 | 1 | | 152-167 | | 1 |
| A/Beijing/353/89 | H3N2 | | | 1 | | 91-97 | 98-106 | 1 | | 136-141 | | 1 |
| A/Europe/C2-5/02 | H3N2 | | | 1 | | 91-97 | 98-106 | | 1 | 136-141 | | |
| A/Finland/C2-10/02 | H3N2 | | | 1 | | 91-97 | 98-106 | | 1 | 136-141 | | |
| A/Finland/12/02 | H3N2 | | | 1 | | 91-97 | 98-106 | | 1 | 136-141 | | |
| A/Finland/C2-17/02 | H3N2 | | | 1 | | 91-97 | 98-106 | | 1 | 136-141 | | |
| A/Finland/C2-14/02 | H3N2 | | | 1 | | 91-97 | 98-106 | | 1 | 136-141 | | |
| A/Finland/C2-13/02 | H3N2 | | | 1 | | 91-97 | 98-106 | | 1 | 136-141 | | |
| A/Finland/C2-7/02 | H3N2 | | | 1 | | 91-97 | 98-106 | | 1 | 136-141 | | |
| A/Finland/684/99 | H3N2 | | | 1 | | 91-97 | 98-106 | 1 | | 136-141 | | |
| A/Finland/663/99 | H3N2 | | | 1 | | 91-97 | 98-106 | 1 | | 136-141 | | |
| A/Finland/645/99 | H3N2 | | | 1 | | 91-97 | 98-106 | 1 | | 136-141 | | |
| A/Finland/455/04 | H3N2 | | | 1 | | 91-97 | 98-106 | 1 | | 136-141 | | |
| A/Finland/481/04 | H3N2 | | | 1 | | 91-97 | 98-106 | 1 | | 136-141 | | |
| A/Finland/482/04 | H3N2 | | | 1 | | 91-97 | 98-106 | 1 | | 136-141 | | |
| A/Finland/485/04 | H3N2 | | | 1 | | 91-97 | 98-106 | 1 | | 136-141 | | |
| A/Finland/486/04 | H3N2 | | | 1 | | 91-97 | 98-106 | 1 | | 136-141 | | |
| A/Finland/C4-22/03 | H3N2 | | | 1 | | 91-97 | 98-106 | 1 | | 136-141 | | |
| A/Finland/C4-23/03 | H3N2 | | | 1 | | 91-97 | 98-106 | 1 | | 136-141 | | |
| A/Finland/435/03 | H3N2 | | | 1 | | 91-97 | 98-106 | 1 | | 136-141 | | |
| A/Finland/272/03 | H3N2 | | | 1 | | 91-97 | 98-106 | 1 | | 136-141 | | |
| A/Finland/358/03 | H3N2 | | | 1 | | 91-97 | 98-106 | 1 | | 136-141 | | |
| A/Finland/437/03 | H3N2 | | | 1 | | 91-97 | 98-106 | 1 | | 136-141 | | |
| A/Finland/402/03 | H3N2 | | | 1 | | 91-97 | 98-106 | 1 | | 136-141 | | |
| A/Finland/1/02 | H3N2 | | | 1 | | 91-97 | 98-106 | 1 | | 136-141 | | |

| Virus name | c) | Pos, Past | a) | b) | d) sequence | a) | b) | c) | Prev. Pos. Past |
|---|---|---|---|---|---|---|---|---|---|
| A/X31 | | 146-151NWLTK | 1 | | | | 1 | | YI180-185ST |
| A/Finland/445/96 | | 146-151NWL | | 1 | | 1 | | | YI180-185 |
| A/Finland/539/97 | | 146-151NWL | | 1 | | 1 | | | YI180-185 |
| A/Finland/447/96 | | 146-151NWL | | 1 | | 1 | | | YI180-185 |
| A/Finland/313/03 | 1 | 146-151NWL | | 1 | | 1 | | | YI180-185 |
| A/Finland/594/98 | | 146-151NWL | | 1 | | 1 | | | YI180-185 |
| A/Finland/587/98 | | 146-151NWL | | 1 | | 1 | | | YI180-185 |
| A/Finland/590/98 | | 146-151NWL | | 1 | | 1 | | | YI180-185 |
| A/Finland/528/97 | | 146-151NWL | | 1 | | 1 | | | YI180-185 |
| A/Finland/339/95 | | 146-151NWL | | 1 | | 1 | | | YI180-185 |
| A/Finland/380/95 | | 146-151NWL | | 1 | | 1 | | | YI180-185 |
| A/Finland/364/95 | | 146-151NWL | | 1 | | 1 | | | YI180-185 |
| A/Finland/296/93 | | 146-151NWL | | 1 | | 1 | | | YI180-185 |
| A/Finland/256/93 | | 146-151NWL | | 1 | | 1 | | | YI180-185 |
| A/Finland/321/93 | | 146-151NWL | | 1 | | 1 | | | YI180-185 |
| A/Finland/263/93 | | 146-151NWL | | 1 | | 1 | | | YI180-185 |
| A/Finland/190/92 | | 146-151NWL | | 1 | | 1 | | | YI180-185 |
| A/Finland/218/92 | | 146-151NWL | | 1 | | 1 | | | YI180-185 |
| A/Finland/191/92 | | 146-151NWL | | 1 | | 1 | | | YI180-185 |
| A/Finland/110/89 | | 146-151NWL | | 1 | | 1 | | | YI180-185 |
| A/Finland/220/92 | | 162-167NWL | | 1 | | 1 | | | YI196-201 |
| A/Finland/218/92 | | 162-167NWL | | 1 | | 1 | | | YI196-201 |
| A/Beijing/353/89 | | 146-151NWL | | 1 | | 1 | | | YI180-185 |
| A/Europe/C2-5/02 | 1 | 146-151NWL | | 1 | | 1 | | | YI180-185 |
| A/Finland/C2-10/02 | 1 | 146-151NWL | | 1 | | 1 | | | YI180-185 |
| A/Finland/12/02 | 1 | 146-151NWL | | 1 | WVGLHP | | | | YI180-185 |
| A/Finland/C2-17/02 | 1 | 146-151NWL | | 1 | | 1 | | | YI180-185 |
| A/Finland/C2-14/02 | 1 | 146-151NWL | | 1 | | 1 | | | YI180-185 |
| A/Finland/C2-13/02 | 1 | 146-151NWL | | 1 | | 1 | | | YI180-185 |
| A/Finland/C2-7/02 | 1 | 146-151NWL | | 1 | | 1 | | | YI180-185 |
| A/Finland/684/99 | 1 | 146-151NWL | | 1 | | 1 | | | YI180-185 |
| A/Finland/663/99 | 1 | 146-151NWL | | 1 | | 1 | | | YI180-185 |
| A/Finland/645/99 | 1 | 146-151NWL | | 1 | | 1 | | | YI180-185 |
| A/Finland/455/04 | 1 | 146-151NWL | | 1 | | 1 | | | YI180-185 |
| A/Finland/481/04 | 1 | 146-151NWL | | 1 | | 1 | | | YI180-185 |
| A/Finland/482/04 | 1 | 146-151NWL | | 1 | | 1 | | | YI180-185 |
| A/Finland/485/04 | 1 | 146-151NWL | | 1 | | 1 | | | YI180-185 |
| A/Finland/486/04 | 1 | 146-151NWL | | 1 | | 1 | | | YI180-185 |
| A/Finland/C4-22/03 | 1 | 146-151NWL | | 1 | | 1 | | | YI180-185 |
| A/Finland/C4-23/03 | 1 | 146-151NWL | | 1 | | 1 | | | YI180-185 |
| A/Finland/435/03 | 1 | 146-151NWL | | 1 | | 1 | | | YI180-185 |
| A/Finland/272/03 | 1 | 146-151NWL | | 1 | | 1 | | | YI180-185 |
| A/Finland/358/03 | 1 | 146-151NWL | | 1 | | 1 | | | YI180-185 |
| A/Finland/437/03 | 1 | 146-151NWL | | 1 | | 1 | | | YI180-185 |
| A/Finland/402/03 | 1 | 146-151NWL | | 1 | | 1 | | | YI180-185 |
| A/Finland/1/02 | 1 | 146-151NWL | | 1 | | 1 | | | YI180-185 |

TABLE 10
PEPTIDE EPITOPES 1-3 in current and former (older) H3 viruses

| | Peptide 1 | Peptide 2 | Peptide 3 | Control |
|---|---|---|---|---|
| | SKAYSNC | SYACKR | RPWVRGV/L/I | YPYDVPDYA/V |
| A/X31, old virus | − | − | + | + |
| | (SKAFSNC) | (SNACKR) | | |
| H3N2 Finland viruses 1989- (20) | | | | |
| A/Finland/110, 353/89 | + | + | + | + |
| A/Finland/190, 191, 218, 220/92 | + | + | + | + |
| A/Finland/256, 263, 296, 321/93 | + | + | + | + |
| A/Finland/339, 364, 380/95 | + | + | + | + |
| A/Finland/528/97 | + | + | + | + |
| A/Finland/587, 590, 594/98 | + | + | + | + |
| A/Finland/445, 447/96 | − | + | + | + |
| A/Finland/539/97 | − | + | + | + |
| | (STAYSN/DC) | | | |
| H3N2 Finland viruses 1999- (24) | | | | |
| A/Finland/645, 663, 684/99 | + | − | + | + |
| A/Europe/C2-5/02 | + | − | + | + |
| A/Finland/12, C2-7, 10, 13, 14, 17/02 | + | − | + | + |
| A/Finland/1/02 | + | − | − | + |
| A/Finland/272, 358, 402, 435, 437, C4-22, 23/03 | + | − | − | + |
| A/Finland/455, 481-2, 485-6/04 | + | − | − | + |
| A/Finland/313/03 | − | − | − | + |
| | (SKADSNC) | (SSACKR) | (RPRVRD(V/I/X)) | |

### REFERENCES

Glick G D, et al, (1991) J. of Biological Chemistry 266(35):23660-23669
Hennecke J, et al, (2000) The EMBO Journal 19(21):5611-5624
Lin A H & Cannon P M (2002) Virus Res. 83(1-2):43-56
Lu Y, et al (2002) Int Arch Allergy Immunol. 127(3):245-250
Sauter N K, et al (1992) Proc. Natl. Acad. Sci. USA 89:324-328
Suzuki Y, et al (1992) Virology 189:121-131

1. A method for evaluating the potential of a chemical entity to bind to a peptide epitope derived from the divalent sialoside binding site of hemagglutinin protein of influenza virus comprising the steps of:
(i) contacting said chemical entity with said peptide under conditions that allow said chemical entity to bind said peptide; and
(ii) detecting the presence of a complex of said chemical entity and said peptide;
wherein said peptide epitope is peptide 1 corresponding to cysteine 97 region, and/or peptide 2 corresponding to cysteine 139 region and/or peptide 3 corresponding to the region of amino acids 220-226 as defined by the amino acid sequence of X31-hemaglutinin and
wherein
said peptide epitope comprises
a) a conformational peptide epitope, comprising at least one cysteine residue or cysteine analogous amino acid residue conjugated from the side chain and the peptide epitope comprises less than 100 amino acid residues, preferably less than 30 amino acid residues present in a natural influenza virus peptide or
b) the conformational peptide epitope is a short peptide epitope comprising 3 to 12 amino acid residues, preferably comprising less than 12 amino acid residues, more preferably less than 11 amino acid residue.

2-5. (canceled)

6. The method according to claim 1, wherein the conformational peptide epitope is
i) peptide 1 or peptide 2 conjugated from a cysteine or cysteine analogous residue side chain of the peptide epitope or
ii) peptide 3, which is in a cyclic form via a bridge formed by adding cysteine residues or cysteine analogous residues to the peptide sequence to form a loop comprising conformation similar to a peptide loop on the surface of hemagglutinin protein.

7. (canceled)

8. The method according to claim 1, wherein said peptide is selected from the group consisting of peptide 2 epitope cores including TSSACKR(R), TSSACIR(R), SS SACKR(R), (G)VTAACSH, (G)VTASCSH, (G)VSASCSH, GSNACKR, GSYACKR and GSSACKR or group consisting of peptide 3 epitope cores including RPRVRNI(P), RPKVRDQ, RPKVNGQ, RPRVRD(V/I/X)(P), RPRIRNI(P), RPWVRGL.

9. (canceled)

10. A conformational antigenic peptide or peptide composition comprising at least one peptide as described in claim 1, preferably comprising peptide 2 or peptide 3.

11. The antigenic peptide composition according to claim 10 comprising at least two peptides selected from the group peptide 1, peptide 2 and peptide 3, optionally at least two peptides from the group: peptide 2 and peptide 3.

12-13. (canceled)

14. The method according to claim 1 wherein the method is used for selection of chemical entities, preferably antibodies, preferably from a library of the entities and the selection is performed in vivo, ex vivo or in vitro and optionally the detection is observing the result of the selection, optionally wherein the method involves specific conjugation of the peptide to matrix by a covalent bond or strong non-covalent interaction, and wherein covalent bond is formed from sulphur atom of a cysteine residue, optionally to maleimide or analogous structure or to a sulphur of cysteine in the matrix or the strong non-covalent interaction is binding of a ligand to a protein, optionally biotin binding to an avidin protein, optionally the peptide is biotinylated.

15-20. (canceled)

21. The method according to claim 1, wherein said peptide is selected from the group consisting of KVR-region peptides of hemagglutinin type 1, WVR-region peptides of hemagglutinin type 3, KVN-region peptides of hemagglutinin type 5, TSNSENGT(C)-region of hemagglutinin type 1, SKAFSN(C)-region peptides of hemagglutinin type 3, KXNPVNXL(C)-region of hemagglutinin type 5, TTKGVTAA(C)-region of hemagglutinin type 1, GGSNA-region peptides of hemagglutinin type 3, and DASSGVSSA(C)PY-region of hemagglutinin type 5.

22-25. (canceled)

26. The method according to the claim 1 for producing a peptide vaccine against influenza comprising steps of:
preparing said peptide conjugate
administering said peptide conjugate to an animal; and
monitoring the animal in order to detect immune response against the peptide

27. (canceled)

28. The peptide conjugate according to claim 10 comprising a carrier, other immunogenic peptides, or an adjuvant, wherein said peptide is optionally covalently linked to the surface of a carrier protein and wherein said peptide is preferably a peptide set forth in SEQ ID NO:12.

29-32. (canceled)

33. The method according to the claim 1, further including evaluating the potential of a chemical entity to bind to:
a) a molecule or molecular complex comprising a large binding site defined by structure coordinates of influenza hemagglutinin amino acids Tyr98, Gly135, Trp153, His183, Leu194 and Gly225 of Region A; and Ser95, Va1223, Arg224, Gly225 and Asn165 of Region B; and Thr65, Ser71, Glu72, Ser95, Gly98, Pro99, Tyr100 and Arg269 of Region C according to FIG. 1; or
b) a homologue of said molecule or molecular complex, wherein said homologue comprises a binding site that has a root mean square deviation from the backbone atoms of said amino acids of not more than 1.5 Å comprising the steps of:
(i) employing computational means to perform a fitting operation between the chemical entity and the large binding site of the molecule or molecular complex; and
(ii) analyzing the results of said fitting operation to quantify the association between the chemical entity and the large binding site
and wherein said large binding site is optionally further defined by at least one of the structure coordinates of influenza hemagglutinin semi- or nonconserved amino acids Gly134, Asn137, Ala138, Thr155, Glu190 and Leu226 of Region A; Phe94, Asn96, Asn137, Ala138, Lys140 and Arg207 of Region B; Ser91, Ala 93, Tyr105 and Arg208 of Region C.

34-37. (canceled)

38. The method according to the claim 1, for identifying a modulator of binding between the large binding site of influenza hemagglutinin and its ligand divalent sialoside, comprising steps of:
(a) contacting the large binding site of influenza hemagglutinin and its ligand in the presence and in the absence of a putative modulator compound;
(b) detecting binding between the large binding site of influenza hemagglutinin and its ligand in the presence and absence of the putative modulator; and
(c) identifying a modulator compound in view of decreased or increased binding between the large binding site of influenza hemagglutinin and its ligand in the presence of the putative modulator, as compared to binding in the absence of the putative modulator, wherein the modulator binds to peptide epitope according to claim 1.

39. (canceled)

40. The method according to the claim 1, for selecting peptide epitopes for immunization and developing peptide vaccines against influenza comprising at least one di- to decapeptide epitope of the large binding site described in Table 1, wherein the method involves analysis according to the claim 1 for antibody as a chemical entity blocking the large binding site.

41. (canceled)

42. The method according to claim 1, using peptide 1, peptide 2 or peptide 3 selected from the group consisting of
K₁V₂R₃, W₁V₂R₃, K₁V₂N₃,
T₁P₂N₃P₄E₅N₆G₇T₈, S₁K₂A₃Y₄S₅N₆, K₁A₂N₃P₄A₅N₆D₇L₈,
V₁T₂K₃G₄V₅S₆A₇S₈, G₁T₂S₃S_{A}A₅, E₁A₂S₃S₄G₅V₆S₇S₈A₉, and said peptide corresponding to influenza virus A hemagglutinin.

43. The antigenic compound according to the claim 10 comprising a peptide selected from the group consisting of
K₁V₂R₃, W₁V₂R₃, K₁V₂N₃,
T₁P₂N₃P₄E₅N₆G₇T₈, S₁K₂A₃Y₄S₅N₆, K₁A₂N₃P₄A₅N₆D₇L₈,
V₁T₂K₃G₄V₅S₆A₇S₈, G₁T₂S₃S₄A₅, E₁A₂S₃S₄G₅V₆S₇S₈A₉, and said peptide corresponds to influenza virus A hemagglutinin.

44-56. (canceled)

57. The antigenic compound according to claim 1, wherein said antigenic compound comprises at least two peptides as defined in claim 1.

58-68. (canceled)

69. An isolated nucleotide encoding an antigenic compound as described in claim 1, the substance optionally being a primer.

70. The nucleotide according to the claim 69 for use in the method for detecting nucleic acid encoding antigenic compound according to claim 43 in a sample comprising:
amplifying DNA reverse transcribed from RNA obtained from the sample using one or more primers each comprising any one of the sequences as listed in Table 1 or sequences in FIGS. 17-19;
and detecting a product of amplification, wherein the presence of the product of amplification indicates the presence of an influenza virus hemagglutinin in the sample.

71-77. (canceled)

78. The nucleotide according to the claim 69, for a use of detecting nucleic acid encoding antigenic compound according to claim 1 in a sample comprising:
contacting the sample with a primer immobilized on a support, said primer comprising a sequence listed in Table 1 or sequences in FIGS. 17-19, under conditions suitable for hybridizing the primer and the sample; and
detecting hybridization of the primer and the sample or
for determining nucleic acid or amino acid sequence of the divalent sialoside binding site of a hemagglutinin protein of influenza virus comprising the steps of:
(a) isolating genomic nucleic acid of an influenza virus; and
(b) sequencing a nucleic acid sequence encoding the cysteine 97 region, cysteine 139 region and the region of amino acids 220-226 as defined by the amino acid sequence of X31-hemaglutinin,
wherein said method optionally comprises a further step of designing peptides for influenza vaccine development based on the sequencing results obtained in step (b) and optionally
the use further including contacting the sample with a nucleic acid microarray, the nucleic acid microarray comprising one or more primers, each of said primers comprising a sequence of any one listed in Table 1 or sequences in FIGS. 17-19, under conditions suitable for hybridizing the one or more primers and the sample;
and detecting hybridization of the one or more primers and the sample,
or optionally
the nucleotide being part of a nucleic acid microarray comprising a primer, said primer comprising a sequence of any one of Table 1 or sequences in FIGS. 17-19 or a kit comprising a primer and/or nucleic acid as defined above and instructions for detecting antigenic compound according to claim 1.

79-87. (canceled)

88. The method according to the claim 1, for identifying influenza virus in a biological sample, the method comprising: (a) contacting the biological sample with an antibody substance capable of binding antigenic compound according to claim 1; and (b) detecting the binding between said antibody substance and antigenic compound in the sample, said binding indicating the presence and type of influenza virus in the sample.

89-96. (canceled)

97. The substance according to claim 10, wherein the substance is according to Formula
[PEP-(y)ₚ-(S)_{q}-(z)ᵣ-]ₙPO (SP1)
wherein PO is an oligomeric or polymeric carrier structure, PEP is the peptide epitope sequence as defined for Peptide 1, Peptide 2 and Peptide 3, PO is preferably selected from the group consisting of: a) solid phases, b) immunogenic and or oligomeric or polymeric carrier such as multiple antigen presenting (MAP) constructs, proteins such as KLH (keyhole limpet hemocyanin oligosaccharide or polysaccharide structure, n is an integer>1 indicating the number of PEP groups covalently attached to the carrier PO, S is a spacer group, p, q and r are each 0 or 1, whereby at least one of p and r is different from 0, y and z are linking groups, at least one of y and z being a linking atom group also referred as “chemoselective ligation group”, in a preferred embodiment comprising at least one an O-hydroxylamine residue —O—NH— or —O—N═, with the nitrogen atom being linked to the OS and/or PO structure, respectively, and the other y and z, if present, is a chemoselective ligation group, with the proviso that when n is 1, the carrier structure is a monovalent immunogenic carrier.


SEQUENCE LISTING (32 sequences)

SEQ ID NO: 1 - protein, 8 aa, Influenza virus
  MISC_FEATURE (8)..(8): Xaa is Arg or nothing
TSSACKRX

SEQ ID NO: 2 - protein, 8 aa, Influenza virus
  MISC_FEATURE (8)..(8): Xaa is Arg or nothing
TSSACIRX

SEQ ID NO: 3 - protein, 8 aa, Influenza virus
  MISC_FEATURE (8)..(8): Xaa is Arg or nothing
SSSACKRX

SEQ ID NO: 4 - protein, 8 aa, Influenza virus
  MISC_FEATURE (1)..(1): Xaa is Gly or nothing
XVTAACSH

SEQ ID NO: 5 - protein, 8 aa, Influenza virus
  MISC_FEATURE (1)..(1): Xaa is Gly or nothing
XVTASCSH

SEQ ID NO: 6 - protein, 8 aa, Influenza virus
  MISC_FEATURE (1)..(1): Xaa is Gly or nothing
XVSASCSH

SEQ ID NO: 7 - protein, 7 aa, Influenza virus
GSNACKR

SEQ ID NO: 8 - protein, 7 aa, Influenza virus
GSYACKR

SEQ ID NO: 9 - protein, 7 aa, Influenza virus
GSSACKR

SEQ ID NO: 10 - protein, 8 aa, Influenza virus
  MISC_FEATURE (8)..(8): Xaa is Pro or nothing
RPRVRNIX

SEQ ID NO: 11 - protein, 7 aa, Influenza virus
RPKVRDQ

SEQ ID NO: 12 - protein, 6 aa, Influenza virus
  MISC_FEATURE (3)..(3): Xaa can be Lys, Glu, Trp, or Arg
RPXVXX

SEQ ID NO: 13 - protein, 7 aa, Influenza virus
RPKVNGQ

SEQ ID NO: 14 - protein, 8 aa, Influenza virus
  MISC_FEATURE (7)..(7): Xaa is Val, Ile or nothing
RPRVRDXX

SEQ ID NO: 15 - protein, 8 aa, Influenza virus
  MISC_FEATURE (8)..(8): Xaa is Pro or nothing
RPRIRNIX

SEQ ID NO: 16 - protein, 7 aa, Influenza virus
RPWVRGL

SEQ ID NO: 17 - protein, 9 aa, Influenza virus
  MISC_FEATURE (9)..(9): Xaa is Cys or nothing
TSNSENGTX

SEQ ID NO: 18 - protein, 7 aa, Influenza virus
  MISC_FEATURE (7)..(7): Xaa is Cys or nothing
SKAFSNX

SEQ ID NO: 19 - protein, 9 aa, Influenza virus
  MISC_FEATURE (2)..(2): Xaa is Ala, Thr, Pro, Ile, Val, Asp or Asn
KXNPVNXLX

SEQ ID NO: 20 - protein, 9 aa, Influenza virus
  MISC_FEATURE (9)..(9): Xaa is Cys or nothing
TTKGVTAAX

SEQ ID NO: 21 - protein, 5 aa, Influenza virus
GGSNA

SEQ ID NO: 22 - protein, 12 aa, Influenza virus
  MISC_FEATURE (10)..(10): Xaa is Cys or nothing
DASSGVSSAXPY

SEQ ID NO: 23 - protein, 8 aa, Influenza virus
TPNPENGT

SEQ ID NO: 24 - protein, 8 aa, Influenza virus
RSNAENGN

SEQ ID NO: 25 - protein, 6 aa, Influenza virus
SKAYSN

SEQ ID NO: 26 - protein, 6 aa, Influenza virus
SNAFSN

SEQ ID NO: 27 - protein, 8 aa, Influenza virus
KANPANDL

SEQ ID NO: 28 - protein, 8 aa, Influenza virus
VTKGVSAS

SEQ ID NO: 29 - protein, 8 aa, Influenza virus
QTGGVSAA

SEQ ID NO: 30 - protein, 9 aa, Influenza virus
EASSGVSSA

SEQ ID NO: 31 - protein, 5 aa, Influenza virus
GTSSA

SEQ ID NO: 32 - protein, 5 aa, Influenza virus
GTSYA